SEQUENCE LISTING (141 sequences)

SEQ ID NO: 1 - DNA, 354 bp, Artificial Sequence
  Synthetic
caggtgcagctggtggagtctgggggaggcttggtcaagcctggagggtccctgagactctcctgtgcagcctctggattcaccttcagtgaatactacctgacctggatccgccaggctccagggaaggggctggactggattgcatacattagtagtagtggttacaatatatattacgcagactctgtgaaggaccgattcaccatttccagggacaacggcaagaactcactgtttctgcaaatgaacaacctgagagccgaagacacggccgtctatttttgtgcgagagagggtgtaacggacggtatggacgtctggggccaagggaccacggtcaccgtctcctca

SEQ ID NO: 2 - protein, 118 aa, Artificial Sequence
  Synthetic
QVQLVESGGGLVKPGGSLRLSCAASGFTFSEYYLTWIRQAPGKGLDWIAYISSSGYNIYYADSVKDRFTISRDNGKNSLFLQMNNLRAEDTAVYFCAREGVTDGMDVWGQGTTVTVSS

SEQ ID NO: 3 - DNA, 24 bp, Artificial Sequence
  Synthetic
ggattcaccttcagtgaatactac

SEQ ID NO: 4 - protein, 8 aa, Artificial Sequence
  Synthetic
GFTFSEYY

SEQ ID NO: 5 - DNA, 24 bp, Artificial Sequence
  Synthetic
attagtagtagtggttacaatata

SEQ ID NO: 6 - protein, 8 aa, Artificial Sequence
  Synthetic
ISSSGYNI

SEQ ID NO: 7 - DNA, 33 bp, Artificial Sequence
  Synthetic
gcgagagagggtgtaacggacggtatggacgtc

SEQ ID NO: 8 - protein, 11 aa, Artificial Sequence
  Synthetic
AREGVTDGMDV

SEQ ID NO: 9 - DNA, 324 bp, Artificial Sequence
  Synthetic
gacatccagatgacccagtctccatcctccctgtctgcatctgtaggagacagagtcaccatcacttgccgggcaagtcagagcatcgacacctttttaaattggtatcagcagaagccagggaaagtccctaaactcctgatctatgctgcatccaatttggaaagtggggtcccatcaaggttcagtggcagtggatctgggacagttttcactctcaccatcagccggctgcaacctgaagattttgcaacttactactgtcaacagagttacagtattcctccgatcaccttcggccaagggacacgagtggagattaaa

SEQ ID NO: 10 - protein, 108 aa, Artificial Sequence
  Synthetic
DIQMTQSPSSLSASVGDRVTITCRASQSIDTFLNWYQQKPGKVPKLLIYAASNLESGVPSRFSGSGSGTVFTLTISRLQPEDFATYYCQQSYSIPPITFGQGTRVEIK

SEQ ID NO: 11 - DNA, 18 bp, Artificial Sequence
  Synthetic
cagagcatcgacaccttt

SEQ ID NO: 12 - protein, 6 aa, Artificial Sequence
  Synthetic
QSIDTF

SEQ ID NO: 13 - DNA, 9 bp, Artificial Sequence
  Synthetic
gctgcatcc

SEQ ID NO: 14 - protein, 3 aa, Artificial Sequence
  Synthetic
AAS

SEQ ID NO: 15 - DNA, 30 bp, Artificial Sequence
  Synthetic
caacagagttacagtattcctccgatcacc

SEQ ID NO: 16 - protein, 10 aa, Artificial Sequence
  Synthetic
QQSYSIPPIT

SEQ ID NO: 17 - DNA, 1335 bp, Artificial Sequence
  Synthetic
caggtgcagctggtggagtctgggggaggcttggtcaagcctggagggtccctgagactctcctgtgcagcctctggattcaccttcagtgaatactacctgacctggatccgccaggctccagggaaggggctggactggattgcatacattagtagtagtggttacaatatatattacgcagactctgtgaaggaccgattcaccatttccagggacaacggcaagaactcactgtttctgcaaatgaacaacctgagagccgaagacacggccgtctatttttgtgcgagagagggtgtaacggacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 444 aa, Artificial Sequence
  Synthetic
QVQLVESGGGLVKPGGSLRLSCAASGFTFSEYYLTWIRQAPGKGLDWIAYISSSGYNIYYADSVKDRFTISRDNGKNSLFLQMNNLRAEDTAVYFCAREGVTDGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 648 bp, Artificial Sequence
  Synthetic
gacatccagatgacccagtctccatcctccctgtctgcatctgtaggagacagagtcaccatcacttgccgggcaagtcagagcatcgacacctttttaaattggtatcagcagaagccagggaaagtccctaaactcctgatctatgctgcatccaatttggaaagtggggtcccatcaaggttcagtggcagtggatctgggacagttttcactctcaccatcagccggctgcaacctgaagattttgcaacttactactgtcaacagagttacagtattcctccgatcaccttcggccaagggacacgagtggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 215 aa, Artificial Sequence
  Synthetic
DIQMTQSPSSLSASVGDRVTITCRASQSIDTFLNWYQQKPGKVPKLLIYAASNLESGVPSRFSGSGSGTVFTLTISRLQPEDFATYYCQQSYSIPPITFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 1413 bp, Artificial Sequence
  Synthetic
gacatccagatgacccagtctccatcctccctgtctgcatctgtaggagacagagtcaccatcacttgccgggcaagtcagagcatcgacacctttttaaattggtatcagcagaagccagggaaagtccctaaactcctgatctatgctgcatccaatttggaaagtggggtcccatcaaggttcagtggcagtggatctgggacagttttcactctcaccatcagccggctgcaacctgaagattttgcaacttactactgtcaacagagttacagtattcctccgatcaccttcggccaagggacacgagtggagattaaacgaggtggaggcggtagtggcggaggcggaagtggtggaggaggctcacaggtgcagctggtggagtctgggggaggcttggtcaagcctggagggtccctgagactctcctgtgcagcctctggattcaccttcagtgaatactacctgacctggatccgccaggctccagggaaggggctggactggattgcatacattagtagtagtggttacaatatatattacgcagactctgtgaaggaccgattcaccatttccagggacaacggcaagaactcactgtttctgcaaatgaacaacctgagagccgaagacacggccgtctatttttgtgcgagagagggtgtaacggacggtatggacgtctggggccaagggaccacggtcaccgtctcctcaggaggtggtggaagtactaccactcctgctccccgccccccaacacctgctccaactattgcatcccaaccactctccctcagacccgaagcttgtcgccccgccgccggaggtgctgttcacactagaggactcgattttgcttgcgacatttatatctgggccccacttgcaggtacttgcggagtattgctgctctcacttgttattactctttattgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 22 - protein, 470 aa, Artificial Sequence
  Synthetic
DIQMTQSPSSLSASVGDRVTITCRASQSIDTFLNWYQQKPGKVPKLLIYAASNLESGVPSRFSGSGSGTVFTLTISRLQPEDFATYYCQQSYSIPPITFGQGTRVEIKRGGGGSGGGGSGGGGSQVQLVESGGGLVKPGGSLRLSCAASGFTFSEYYLTWIRQAPGKGLDWIAYISSSGYNIYYADSVKDRFTISRDNGKNSLFLQMNNLRAEDTAVYFCAREGVTDGMDVWGQGTTVTVSSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 5 aa, Artificial Sequence
  Synthetic
GGGGS

SEQ ID NO: 24 - protein, 10 aa, Artificial Sequence
  Synthetic
GGGGSGGGGS

SEQ ID NO: 25 - protein, 15 aa, Artificial Sequence
  Synthetic
GGGGSGGGGSGGGGS

SEQ ID NO: 26 - protein, 18 aa, Artificial Sequence
  Synthetic
GSTSGSGKPGSGEGSTKG

SEQ ID NO: 27 - protein, 45 aa, Artificial Sequence
  Synthetic
TTTPAPRPPTPAPTIASQPLSLRPEACRPAAGGAVHTRGLDFACD

SEQ ID NO: 28 - protein, 24 aa, Artificial Sequence
  Synthetic
IYIWAPLAGTCGVLLLSLVITLYC

SEQ ID NO: 29 - protein, 42 aa, Artificial Sequence
  Synthetic
KRGRKKLLYIFKQPFMRPVQTTQEEDGCSCRFPEEEEGGCEL

SEQ ID NO: 30 - protein, 112 aa, Artificial Sequence
  Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 1724 bp, Artificial Sequence
  Synthetic
agagacaagcgagcttctgcgtctgactcgcagcttgagactggcggagggaagcccgcccaggctctataaggagacaaggttctgagcagacaggccaaccggaggacaggattccctggaggccacagaggagcaccaaggagaagatctgcctgtgggtccccattgcccagcttttgcctgcactcttgcctgctgccctgaccagagtcatcatgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtctgagcatgagttgcagccagggctgtggggaaggggcagggctgggccagtgcatctaacagccctgtgcagcagcttcccttgcctcgtgtaacatgaggcccattcttcactctgtttgaagaaaatagtcagtgttcttagtagtgggtttctattttgttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaataggtgagataaattaaaagatacttaattcccgccttatgcctcagtctattctgtaaaatttaaaaaatatatatgcatacctggatttccttggcttcgtgaatgtaagagaaattaaatctgaataaataattctttctgttaa

SEQ ID NO: 32 - protein, 317 aa, Artificial Sequence
  Synthetic
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 33 - protein, 9 aa, Artificial Sequence
  Synthetic
KVLEHVVRV

SEQ ID NO: 34 - protein, 39 aa, Artificial Sequence
  Synthetic
IEVMYPPPYLDNEKSNGTIIHVKGKHLCPSPLFPGPSKP

SEQ ID NO: 35 - DNA, 117 bp, Artificial Sequence
  Synthetic
atcgaagtgatgtacccccctccatatctggataacgagaagagcaatggcacaatcatccacgtgaagggcaagcacctgtgcccttctccactgttccccggccctagcaagccc

SEQ ID NO: 36 - protein, 27 aa, Artificial Sequence
  Synthetic
FWVLVVVGGVLACYSLLVTVAFIIFWV

SEQ ID NO: 37 - DNA, 81 bp, Artificial Sequence
  Synthetic
ttttgggtgctggtggtggtgggaggcgtgctggcctgttactccctgctggtgaccgtggccttcatcatcttttgggtg

SEQ ID NO: 38 - protein, 41 aa, Artificial Sequence
  Synthetic
RSKRSRLLHSDYMNMTPRRPGPTRKHYQPYAPPRDFAAYRS

SEQ ID NO: 39 - DNA, 123 bp, Artificial Sequence
  Synthetic
agaagcaagagatccaggctgctgcactctgactatatgaatatgacccctaggcgcccaggccccacaagaaagcactaccagccatatgcaccacctagggacttcgcagcataccgcagc

SEQ ID NO: 40 - protein, 9 aa, Artificial Sequence
  Synthetic
KVREEVVTV

SEQ ID NO: 41 - protein, 9 aa, Artificial Sequence
  Synthetic
KVMCHVRRV

SEQ ID NO: 42 - protein, 9 aa, Artificial Sequence
  Synthetic
KVLERVNAV

SEQ ID NO: 43 - protein, 9 aa, Artificial Sequence
  Synthetic
KVLVEVVDV

SEQ ID NO: 44 - protein, 9 aa, Artificial Sequence
  Synthetic
KVLEKCNRV

SEQ ID NO: 45 - protein, 9 aa, Artificial Sequence
  Synthetic
KVLEHVPLL

SEQ ID NO: 46 - protein, 9 aa, Artificial Sequence
  Synthetic
FLLETVVRV

SEQ ID NO: 47 - protein, 9 aa, Artificial Sequence
  Synthetic
KVLGIVVGV

SEQ ID NO: 48 - protein, 9 aa, Artificial Sequence
  Synthetic
MVLEHPARV

SEQ ID NO: 49 - protein, 9 aa, Artificial Sequence
  Synthetic
KVLEQPVVV

SEQ ID NO: 50 - protein, 9 aa, Artificial Sequence
  Synthetic
KVLQNVLRV

SEQ ID NO: 51 - protein, 9 aa, Artificial Sequence
  Synthetic
KVLEGVVAA

SEQ ID NO: 52 - protein, 9 aa, Artificial Sequence
  Synthetic
AVEEHVVSV

SEQ ID NO: 53 - protein, 9 aa, Artificial Sequence
  Synthetic
KVLETLVTV

SEQ ID NO: 54 - DNA, 372 bp, Artificial Sequence
  Synthetic
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagc

SEQ ID NO: 55 - protein, 124 aa, Artificial Sequence
  Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 56 - DNA, 24 bp, Artificial Sequence
  Synthetic
gggtttacattcgacgattacagc

SEQ ID NO: 57 - protein, 8 aa, Artificial Sequence
  Synthetic
GFTFDDYS

SEQ ID NO: 58 - DNA, 24 bp, Artificial Sequence
  Synthetic
atatcatggaactcaggaagcaag

SEQ ID NO: 59 - protein, 8 aa, Artificial Sequence
  Synthetic
ISWNSGSK

SEQ ID NO: 60 - DNA, 51 bp, Artificial Sequence
  Synthetic
gcaaaatacggcagtggttatggcaagttttatcattatggactggacgtg

SEQ ID NO: 61 - protein, 17 aa, Artificial Sequence
  Synthetic
AKYGSGYGKFYHYGLDV

SEQ ID NO: 62 - DNA, 324 bp, Artificial Sequence
  Synthetic
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 63 - protein, 108 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 64 - DNA, 18 bp, Artificial Sequence
  Synthetic
cagagcattagcagctat

SEQ ID NO: 65 - protein, 6 aa, Artificial Sequence
  Synthetic
QSISSY

SEQ ID NO: 66 - DNA, 30 bp, Artificial Sequence
  Synthetic
caacagagttacagtacccctccgatcacc

SEQ ID NO: 67 - protein, 10 aa, Artificial Sequence
  Synthetic
QQSYSTPPIT

SEQ ID NO: 68 - DNA, 1353 bp, Artificial Sequence
  Synthetic
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagcgccagcacaaaaggtcctagcgtttttccacttgccccatgttcaaggtcaacctccgaaagtaccgccgctcttggctgtctcgtaaaagattattttcccgaacctgtaactgtctcctggaactccggcgcactcacttccggcgtacataccttccccgctgtcctccaatcttccggtctctactccctgtcttctgttgtcactgttccatcatcctcactcggcacaaaaacatatacctgcaacgttgatcacaagccaagtaataccaaagttgataagcgcgtcgaatccaaatacggtcccccctgccccccatgtcccgctccacctgtggctggtccctctgttttcctttttccccctaaacccaaagataccctcatgatttccagaacccccgaggtcacctgcgtcgtcgttgatgtaagccaagaagatcccgaagtccagttcaattggtatgtagacggtgttgaagtccataatgcaaaaacaaaacccagagaggaacagtttaattcaacctatcgtgtcgttagcgtactcaccgttcttcatcaagactggctcaatggaaaagaatataaatgtaaagttagcaacaaaggtctgcccagttcaatcgaaaaaacaattagcaaagccaaaggccaacctcgcgaaccccaagtctataccttgcccccttctcaggaagaaatgaccaaaaaccaagtttcactcacatgcctcgtaaaaggattctatccatcagacattgcagtagaatgggaatctaacggccaacctgaaaataattacaaaaccactcctcctgtcctcgattctgacggctcttttttcctttactccagattgactgttgataaatcccgctggcaggaaggtaacgttttttcttgttctgtgatgcacgaagccctccataacagattcactcaaaaatctctttctctctcccctggcaaataa

SEQ ID NO: 69 - protein, 450 aa, Artificial Sequence
  Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 70 - DNA, 648 bp, Artificial Sequence
  Synthetic
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 71 - protein, 215 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - DNA, 372 bp, Artificial Sequence
  Synthetic
gaagtgcagctggtggagtctgggggaggcttggtgcagcctggcaggtccctgagactctcctgtgcagcctctggattcacctttgctgattataccatgcactgggtccggcaagctccagggaagggcctggagtgggtctcagatattagttggaatagtggtagtatagcctatgcggactctgtgaagggccgattcaccatctccagagacaacgccaagaactccctctatcttcaaatgaacagtctgagaactgaggacacggccttttattactgtgcaaaagatagtaggggctacggtcactataagtacctcggtttggacgtctggggccaagggaccacggtcaccgtctcctca

SEQ ID NO: 73 - protein, 124 aa, Artificial Sequence
  Synthetic
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSS

SEQ ID NO: 74 - DNA, 24 bp, Artificial Sequence
  Synthetic
ggattcacctttgctgattatacc

SEQ ID NO: 75 - protein, 8 aa, Artificial Sequence
  Synthetic
GFTFADYT

SEQ ID NO: 76 - DNA, 24 bp, Artificial Sequence
  Synthetic
attagttggaatagtggtagtata

SEQ ID NO: 77 - protein, 8 aa, Artificial Sequence
  Synthetic
ISWNSGSI

SEQ ID NO: 78 - DNA, 51 bp, Artificial Sequence
  Synthetic
gcaaaagatagtaggggctacggtcactataagtacctcggtttggacgtc

SEQ ID NO: 79 - protein, 17 aa, Artificial Sequence
  Synthetic
AKDSRGYGHYKYLGLDV

SEQ ID NO: 80 - DNA, 1353 bp, Artificial Sequence
  Synthetic
gaagtgcagctggtggagtctgggggaggcttggtgcagcctggcaggtccctgagactctcctgtgcagcctctggattcacctttgctgattataccatgcactgggtccggcaagctccagggaagggcctggagtgggtctcagatattagttggaatagtggtagtatagcctatgcggactctgtgaagggccgattcaccatctccagagacaacgccaagaactccctctatcttcaaatgaacagtctgagaactgaggacacggccttttattactgtgcaaaagatagtaggggctacggtcactataagtacctcggtttggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 81 - protein, 450 aa, Artificial Sequence
  Synthetic
EVQLVESGGGLVQPGRSLRLSCAASGFTFADYTMHWVRQAPGKGLEWVSDISWNSGSIAYADSVKGRFTISRDNAKNSLYLQMNSLRTEDTAFYYCAKDSRGYGHYKYLGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 82 - DNA, 354 bp, Artificial Sequence
  Synthetic
caggtgcagctggtggagtctgggggaggcttggtcaagcctggagggtccctgagactctcctgtgcagcctctggattcaccttcagtgaatactacctgacctggatccgccaggctccagggaaggggctggactggattgcatacattagtagtagtggttacaatatatattacgcagactctgtgaaggaccgattcaccatttccagggacaacggcaacaactcactgtttctgcaaatgaacaacctgagagccgaagacacggccgtctatttttgtgcgagagagggtgtaacggacggtatggacgtctggggccaagggaccacggtcaccgtctcctca

SEQ ID NO: 83 - protein, 118 aa, Artificial Sequence
  Synthetic
QVQLVESGGGLVKPGGSLRLSCAASGFTFSEYYLTWIRQAPGKGLDWIAYISSSGYNIYYADSVKDRFTISRDNGNNSLFLQMNNLRAEDTAVYFCAREGVTDGMDVWGQGTTVTVSS

SEQ ID NO: 84 - DNA, 366 bp, Artificial Sequence
  Synthetic
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggatcacccactacaacccctccctcaagagtcgagtcaccatatcagtagacacgtccaagatccagttctccctgaagctgagttctgtgaccgctgcggacacggccgtgtattactgtgcgagatggggggttcggagggactactactactacggtatggacgtctggggccaagggaccacggtcaccgtctcctca

SEQ ID NO: 85 - protein, 122 aa, Artificial Sequence
  Synthetic
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSS

SEQ ID NO: 86 - DNA, 24 bp, Artificial Sequence
  Synthetic
ggtggctccatcagtagttactac

SEQ ID NO: 87 - protein, 8 aa, Artificial Sequence
  Synthetic
GGSISSYY

SEQ ID NO: 88 - DNA, 21 bp, Artificial Sequence
  Synthetic
atctattacagtgggatcacc

SEQ ID NO: 89 - protein, 7 aa, Artificial Sequence
  Synthetic
IYYSGIT

SEQ ID NO: 90 - DNA, 48 bp, Artificial Sequence
  Synthetic
gcgagatggggggttcggagggactactactactacggtatggacgtc

SEQ ID NO: 91 - protein, 16 aa, Artificial Sequence
  Synthetic
ARWGVRRDYYYYGMDV

SEQ ID NO: 92 - DNA, 324 bp, Artificial Sequence
  Synthetic
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 93 - protein, 108 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 94 - DNA, 21 bp, Artificial Sequence
  Synthetic
cagtcagtctctagctcttat

SEQ ID NO: 95 - protein, 7 aa, Artificial Sequence
  Synthetic
QSVSSSY

SEQ ID NO: 96 - DNA, 9 bp, Artificial Sequence
  Synthetic
ggggcaagt

SEQ ID NO: 97 - protein, 3 aa, Artificial Sequence
  Synthetic
GAS

SEQ ID NO: 98 - DNA, 27 bp, Artificial Sequence
  Synthetic
caacagtacggaagcagcccgtggacg

SEQ ID NO: 99 - protein, 9 aa, Artificial Sequence
  Synthetic
QQYGSSPWT

SEQ ID NO: 100 - DNA, 1347 bp, Artificial Sequence
  Synthetic
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggatcacccactacaacccctccctcaagagtcgagtcaccatatcagtagacacgtccaagatccagttctccctgaagctgagttctgtgaccgctgcggacacggccgtgtattactgtgcgagatggggggttcggagggactactactactacggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 101 - protein, 448 aa, Artificial Sequence
  Synthetic
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 102 - DNA, 648 bp, Artificial Sequence
  Synthetic
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 103 - protein, 215 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - DNA, 1398 bp, Artificial Sequence
  Synthetic
gacatccagatgacccagtctccatcctccctgtctgcatctgtaggagacagagtcaccatcacttgccgggcaagtcagagcatcgacacctttttaaattggtatcagcagaagccagggaaagtccctaaactcctgatctatgctgcatccaatttggaaagtggggtcccatcaaggttcagtggcagtggatctgggacagttttcactctcaccatcagccggctgcaacctgaagattttgcaacttactactgtcaacagagttacagtattcctccgatcaccttcggccaagggacacgagtggagattaaacgaggtggaggcggtagtggcggaggcggaagtggtggaggaggctcacaggtgcagctggtggagtctgggggaggcttggtcaagcctggagggtccctgagactctcctgtgcagcctctggattcaccttcagtgaatactacctgacctggatccgccaggctccagggaaggggctggactggattgcatacattagtagtagtggttacaatatatattacgcagactctgtgaaggaccgattcaccatttccagggacaacggcaagaactcactgtttctgcaaatgaacaacctgagagccgaagacacggccgtctatttttgtgcgagagagggtgtaacggacggtatggacgtctggggccaagggaccacggtcaccgtctcctcaggaggtggtggaagtatcgaagtgatgtacccacccccttatctggataacgagaagagcaatggcacaatcatccacgtgaagggcaagcacctgtgcccctctcctctgttcccaggccccagcaagccattttgggtgctggtggtggtgggaggcgtgctggcctgttactccctgctggtgaccgtggccttcatcatcttttgggtgagatctaagcgcagccggctgctgcactctgactatatgaatatgaccccacggagacctggcccaacaagaaagcactaccagccatatgcaccaccaagggacttcgcagcctacagaagcagggtgaagttttccaggtctgccgatgcaccagcataccagcagggacagaaccagctgtataacgagctgaatctgggcaggcgcgaggagtatgacgtgctggataagaggagaggccgcgatcctgagatgggaggcaagccaaggcgcaagaaccctcaggagggcctgtacaatgagctgcagaaggacaagatggccgaggcctatagcgagatcggcatgaagggagagcggagaaggggcaagggacacgatggcctgtaccagggcctgtccaccgccacaaaggacacctatgatgccctgcacatgcaggccctgcctccaagg

SEQ ID NO: 105 - protein, 466 aa, Artificial Sequence
  Synthetic
DIQMTQSPSSLSASVGDRVTITCRASQSIDTFLNWYQQKPGKVPKLLIYAASNLESGVPSRFSGSGSGTVFTLTISRLQPEDFATYYCQQSYSIPPITFGQGTRVEIKRGGGGSGGGGSGGGGSQVQLVESGGGLVKPGGSLRLSCAASGFTFSEYYLTWIRQAPGKGLDWIAYISSSGYNIYYADSVKDRFTISRDNGKNSLFLQMNNLRAEDTAVYFCAREGVTDGMDVWGQGTTVTVSSGGGGSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - DNA, 381 bp, Artificial Sequence
  Synthetic
gaggtgcagctgttggagtctgggggaggcttggtacagcctggggggtccctgagactcacctgtgcagcctctggattcacctttagaagctatgccatgagctgggtccgccaggctccagggaaggggctggagtgggtctcaactattagtggtaatagtgatagcacatactacgcagactccgtgaagggccggttcaccatctccagagaaaattccaagaacacgctgtatctgcaaatgaacagcctgagagccgaggacacggccgtatattactgtgcgaaagacctccacattactatggttcggggagctatccccgccgatgtttttgatatctggggccaagggacaatggtcaccgtctcttca

SEQ ID NO: 107 - protein, 127 aa, Artificial Sequence
  Synthetic
EVQLLESGGGLVQPGGSLRLTCAASGFTFRSYAMSWVRQAPGKGLEWVSTISGNSDSTYYADSVKGRFTISRENSKNTLYLQMNSLRAEDTAVYYCAKDLHITMVRGAIPADVFDIWGQGTMVTVSS

SEQ ID NO: 108 - DNA, 24 bp, Artificial Sequence
  Synthetic
ggattcacctttagaagctatgcc

SEQ ID NO: 109 - protein, 8 aa, Artificial Sequence
  Synthetic
GFTFRSYA

SEQ ID NO: 110 - DNA, 24 bp, Artificial Sequence
  Synthetic
attagtggtaatagtgatagcaca

SEQ ID NO: 111 - protein, 8 aa, Artificial Sequence
  Synthetic
ISGNSDST

SEQ ID NO: 112 - DNA, 60 bp, Artificial Sequence
  Synthetic
gcgaaagacctccacattactatggttcggggagctatccccgccgatgtttttgatatc

SEQ ID NO: 113 - protein, 20 aa, Artificial Sequence
  Synthetic
AKDLHITMVRGAIPADVFDI

SEQ ID NO: 114 - DNA, 324 bp, Artificial Sequence
  Synthetic
gacatccagatgacccagtctccatcctccctgtctgcatctgtaggagacagagtcaccatcacttgccgggcaagtcagagcattagcttctatttaaattggtatcaacagaaaccagggaaagcccctaagctcctgatctatgctgcatccagtttgcaaagtggggtcccatcaaggttcagtggcagtggatctgagacagatttcactctcaccatcagcagtctgcaacctgaagattttgcaacttactactgtcaacagagttacagtacccctccgatcaccttcggccaagggacacgactggagattaaa

SEQ ID NO: 115 - protein, 108 aa, Artificial Sequence
  Synthetic
DIQMTQSPSSLSASVGDRVTITCRASQSISFYLNWYQQKPGKAPKLLIYAASSLQSGVPSRFSGSGSETDFTLTISSLQPEDFATYYCQQSYSTPPITFGQGTRLEIK

SEQ ID NO: 116 - DNA, 18 bp, Artificial Sequence
  Synthetic
cagagcattagcttctat

SEQ ID NO: 117 - protein, 6 aa, Artificial Sequence
  Synthetic
QSISFY

SEQ ID NO: 118 - DNA, 30 bp, Artificial Sequence; identical to SEQ ID NO: 66
  Synthetic

SEQ ID NO: 119 - protein, 10 aa, Artificial Sequence
  Synthetic
QQSYSTPPIT

SEQ ID NO: 120 - protein, 469 aa, Artificial Sequence
  Synthetic
DIQMTQSPSSLSASVGDRVTITCRASQSIDTFLNWYQQKPGKVPKLLIYAASNLESGVPSRFSGSGSGTVFTLTISRLQPEDFATYYCQQSYSIPPITFGQGTRVEIKGGGGSGGGGSGGGGSQVQLVESGGGLVKPGGSLRLSCAASGFTFSEYYLTWIRQAPGKGLDWIAYISSSGYNIYYADSVKDRFTISRDNGKNSLFLQMNNLRAEDTAVYFCAREGVTDGMDVWGQGTTVTVSSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 465 aa, Artificial Sequence
  Synthetic
DIQMTQSPSSLSASVGDRVTITCRASQSIDTFLNWYQQKPGKVPKLLIYAASNLESGVPSRFSGSGSGTVFTLTISRLQPEDFATYYCQQSYSIPPITFGQGTRVEIKGGGGSGGGGSGGGGSQVQLVESGGGLVKPGGSLRLSCAASGFTFSEYYLTWIRQAPGKGLDWIAYISSSGYNIYYADSVKDRFTISRDNGKNSLFLQMNNLRAEDTAVYFCAREGVTDGMDVWGQGTTVTVSSGGGGSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 10 aa, Artificial Sequence
  Synthetic
GVYDGREHTV

SEQ ID NO: 123 - protein, 10 aa, Artificial Sequence
  Synthetic
GLYDGREHSV

SEQ ID NO: 124 - protein, 10 aa, Artificial Sequence
  Synthetic
GLADGRTHTV

SEQ ID NO: 125 - protein, 10 aa, Artificial Sequence
  Synthetic
GVPDCRIFTV

SEQ ID NO: 126 - protein, 10 aa, Artificial Sequence
  Synthetic
SVYDAREFSV

SEQ ID NO: 127 - protein, 10 aa, Artificial Sequence
  Synthetic
GLSDGQWHTV

SEQ ID NO: 128 - protein, 10 aa, Artificial Sequence
  Synthetic
GVFDNCSHTV

SEQ ID NO: 129 - protein, 10 aa, Artificial Sequence
  Synthetic
KVSDGHFHTV

SEQ ID NO: 130 - protein, 10 aa, Artificial Sequence
  Synthetic
GLYDGMEHLI

SEQ ID NO: 131 - protein, 10 aa, Artificial Sequence
  Synthetic
FLCDPREHLV

SEQ ID NO: 132 - protein, 10 aa, Artificial Sequence
  Synthetic
AVYDGREHTV

SEQ ID NO: 133 - protein, 10 aa, Artificial Sequence
  Synthetic
GAYDGREHTV

SEQ ID NO: 134 - protein, 10 aa, Artificial Sequence
  Synthetic
GVADGREHTV

SEQ ID NO: 135 - protein, 10 aa, Artificial Sequence
  Synthetic
GVYAGREHTV

SEQ ID NO: 136 - protein, 10 aa, Artificial Sequence
  Synthetic
GVYDAREHTV

SEQ ID NO: 137 - protein, 10 aa, Artificial Sequence
  Synthetic
GVYDGAEHTV

SEQ ID NO: 138 - protein, 10 aa, Artificial Sequence
  Synthetic
GVYDGRAHTV

SEQ ID NO: 139 - protein, 10 aa, Artificial Sequence
  Synthetic
GVYDGREATV

SEQ ID NO: 140 - protein, 10 aa, Artificial Sequence
  Synthetic
GVYDGREHAV

SEQ ID NO: 141 - protein, 10 aa, Artificial Sequence
  Synthetic
GVYDGREHTA

(19) United States
(12) Patent Application Publication
(10) Pub. No.: US 2024/0226163 A1
(43) Pub. Date: Jul. 11, 2024

(54) Chimeric Antigen Receptors with MAGE-A4 Specificity and Uses Thereof

(71) Applicant: Regeneron Pharmaceuticals, Inc., Tarrytowm, NY (US)

(72) Inventors: Kevin BRAY, Garnerville, NY (US); Frank DELFINO, Poughquag, NY (US); David DILILLO, New York, NY (US)

(21) Appl. No.: 18/559,009

(22) PCT Filed: May 3, 2022
(86) PCT No.: PCT/US2022/027463

§ 371 Date: Nov. 3, 2023

Related U.S. Application Data
(60) Provisional application No. 63/184,183, filed on May 4, 2021.
(60) Provisional application No. 63/239,293, filed on Aug. 31, 2021.

(51) Int. Cl. A61K 35/17; A61K 39/00; A61P 35/00; C07K 14/725; C07K 16/28; C07K 16/30

(52) U.S. Cl. CPC A61K 35/17; A61K 39/4611; A61K 39/4631; A61K 39/464486; A61P 35/00; C07K 14/7051; C07K 16/2809; C07K 16/30; A61K 2239/13; A61K 2239/28; A61K 2239/29; C07K 2317/31; C07K 2317/53; C07K 2317/565; C07K 2317/622; C07K 2317/73; C07K 2317/92; C07K 2319/03

(57) ABSTRACT

MAGE-A4, or Melanoma-Associated Antigen A4, is a cancer-testis antigen (CTA) on the X chromosome. The present disclosure provides MAGE-A4-specific chimeric antigen receptors, cells expressing such chimeric antigen receptors, and MAGE-A4 specific isolated antibodies. In certain embodiments, engineered cells expressing the chimeric antigen receptors of the present disclosure are capable of inhibiting the growth of tumors expressing MAGE-A4. The engineered cells of the present disclosure are useful for the treatment of diseases and disorders in which an upregulated or induced MAGE-A4-targeted immune response is desired and/or therapeutically beneficial. For example, engineered cells expressing the MAGE-A4-specific chimeric antigen receptors of the present disclosure are useful for the treatment of various cancers.

### CROSS-REFERENCE TO RELATED APPLICATIONS

This application claims the benefit under 35 USC § 119(e) of US Provisional Application Nos.: 63/184,183, filed May 4, 2021 and 63/239,293, filed Aug. 31, 2021, each of which is incorporated herein by reference in its entirety for all purposes.

### REFERENCE TO A SEQUENCE LISTING

This application incorporates by reference the Sequence Listing submitted in Computer Readable Form as file 10901WO01-Sequence.txt, created on May 3, 2022 and containing 95,050 bytes.

### FIELD

The present disclosure provides antibodies, chimeric antigen receptors (CARs), and engineered cells comprising such antibodies and CARs, which are specific for Melanoma-Associated Antigen A4 (MAGE-A4), and methods of use thereof.

### BACKGROUND

MAGE-A4, or Melanoma-Associated Antigen A4, is a cancer-testis antigen (CTA) on the X chromosome. The function of MAGE-A4 is unknown, but it may be involved in cell cycle progression/regulation, transcriptional control, cell survival and/or apoptosis. For example, overexpression of MAGE-A4 has been shown to promote growth of spontaneously transformed oral keratinocytes and inhibit growth arrest of cells in G1.

MAGE-A4 is abundantly expressed by many tumors of different histological types, such as head and neck squamous cell carcinoma, lung carcinoma, such as non-small cell lung carcinoma, esophageal squamous cell carcinoma, colon carcinoma, bladder cancer, mucosal and cutaneous melanomas, ovarian carcinoma, e.g., serous carcinoma, and uterine carcinoma but, in normal healthy adult tissues, MAGE-A4 expression is restricted to the testes.

The ability of MAGE-A4 antigens to elicit immune responses together with its restricted expression pattern have rendered MAGE-A4 a good candidate for cancer immunotherapy.

Dual targeting antibody strategies applied to complex diseases such as cancer also represent one promising strategy, whereby multifactorial modulation aims to improve therapeutic efficacy. CD3 is a homodimeric or heterodimeric antigen expressed on T cells in association with the T cell receptor complex (TCR) and is required for T cell activation. Functional CD3 is formed from the dimeric association of two of four different chains: epsilon, zeta, delta, and gamma. A bispecific antibody having a MAGE-A4 binding arm and a CD3-binding arm may be useful to augment antitumor activity.

Adoptive immunotherapy, which involves the transfer of autologous antigen-specific T cells generated ex vivo, is another promising strategy to treat viral infections and cancer. The T cells used for adoptive immunotherapy can be generated either by expansion of antigen-specific T cells or redirection of T cells through genetic engineering.

Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and heavy chain variable fragments of a monoclonal antibody joined by a flexible linker. The signaling domains for first generation CARs are derived from the cytoplasmic region of the CD3zeta or the Fc receptor gamma chains. First generation CARs have been shown to successfully redirect T-cell cytotoxicity. However, they failed to provide prolonged expansion and anti-tumor activity in vivo. Signaling domains from co-stimulatory molecules, as well as transmembrane and hinge domains have been added to form CARs of second and third generations, leading to some successful therapeutic trials in humans. For example, CAR redirected T cells specific for the B cell differentiation antigen CD19 have shown dramatic efficacy in the treatment of B cell malignancies, while TCR-redirected T cells have shown benefits in patients suffering from solid cancer. Stauss et al. describe strategies to modify therapeutic CARs and TCRs, for use in the treatment of cancer, for example, to enhance the antigen-specific effector function and limit toxicity of engineered T cells (Current Opinion in Pharmacology2015, 24:113-118).

There is an unmet need for new targeting agents based on dual targeting antibody strategies and/or CARs that specifically bind to MAGE-A4 antigens, as well as methods for producing and using such agents in therapeutic and diagnostic settings.

### SUMMARY

In one aspect, the present disclosure provides an antigen-binding protein that specifically binds an HLA-bound Melanoma-Associated Antigen A4 (MAGE-A4), wherein the antigen-binding protein comprises a light chain variable region (LCVR) and a heavy chain variable region (HCVR), wherein the LCVR comprises complementarity determining regions (CDRs) of a LCVR comprising the amino acid sequence of SEQ ID NO: 10 or SEQ ID NO: 115, and wherein the HCVR comprises CDRs of a HCVR comprising the amino acid sequence of SEQ ID NO: 2, SEQ ID NO: 83, or SEQ ID NO: 107.

In some embodiments, the LCVR comprises an amino acid sequence having at least 95% sequence identity to SEQ ID NO: 10 or SEQ ID NO: 115. In some embodiments, the HCVR comprises an amino acid sequence having at least 95% identity to SEQ ID NO: 2, SEQ ID NO: 83, or SEQ ID NO: 107.

In some cases, the antigen-binding protein interacts with amino acids 286-294, or a portion thereof, of SEQ ID NO: 32.

In one aspect, the present disclosure provides a MAGE-A4-specific chimeric antigen receptor (CAR) comprising from N-terminus to C-terminus: (a) an extracellular ligand-binding domain comprising an anti-MAGE-A4 single chain variable fragment (scFv) domain comprising a light chain variable region (LCVR) and a heavy chain variable region (HCVR); (b) a hinge; (c) a transmembrane domain; and (d) a cytoplasmic domain comprising a 4-1BB costimulatory domain or a CD28 costimulatory domain and a CD3zeta signaling domain, wherein the LCVR comprises complementarity determining regions (CDRs) of a LCVR comprising the amino acid sequence of SEQ ID NO: 10 or SEQ ID NO: 115, and CDRs of a HCVR comprising an amino acid sequence of SEQ ID NO: 2, SEQ ID NO: 83 or SEQ ID NO: 107.

In some embodiments, the MAGE-A4-specific CAR comprises, from N-terminus to C-terminus: (a) the extracellular ligand-binding domain; (b) a hinge; (c) a transmembrane domain; and (d) a cytoplasmic domain comprising a costimulatory domain and a signaling domain. In some cases, the anti-MAGE-A4 scFv domain comprises a first linker between the LCVR and the HCVR.

In some embodiments, the MAGE-A4-specific CAR further comprises a second linker between the extracellular ligand-binding domain and the hinge. In some cases, the first linker comprises an amino acid sequence selected from the group consisting of SEQ ID NOs: 23-26, and the second linker comprise amino acid sequences selected from the group consisting of SEQ ID NOs: 23-26. In some cases, the first linker comprises the amino acid sequence of SEQ ID NO: 25 and the second linker comprises the amino acid sequence of SEQ ID NO: 23.

In various embodiments of the MAGE-A4-specific CAR, the hinge, the transmembrane domain, or both, are from a CD8a polypeptide.

In various embodiments of the MAGE-A4-specific CAR, the costimulatory domain comprises a 4-1BB costimulatory domain.

In various embodiments of the MAGE-A4-specific CAR, the costimulatory domain comprises a CD28 costimulatory domain.

In various embodiments of the MAGE-A4-specific CAR, the hinge, the transmembrane domain, or both, are from a CD28 polypeptide.

In various embodiments of the MAGE-A4-specific CAR, the hinge comprises the amino acid sequence of SEQ ID NO: 27.

In various embodiments of the MAGE-A4-specific CAR, the transmembrane domain comprises the amino acid sequence of SEQ ID NO: 28.

In various embodiments of the MAGE-A4-specific CAR, the 4-1BB costimulatory domain comprises the amino acid sequence of SEQ ID NO: 29.

In various embodiments of the MAGE-A4-specific CAR, the hinge comprises the amino acid sequence of SEQ ID NO: 34.

In various embodiments of the MAGE-A4-specific CAR, the transmembrane domain comprises the amino acid sequence of SEQ ID NO: 36.

In various embodiments of the MAGE-A4-specific CAR, the CD28 costimulatory domain comprises the amino acid sequence of SEQ ID NO: 38.

In various embodiments of the MAGE-A4-specific CAR, the signaling domain comprises a CD3zeta signaling domain. In some cases, the CD3zeta signaling domain comprises the amino acid sequence of SEQ ID NO: 30.

In various embodiments, the antigen-binding protein is a MAGE-A4-specific antibody, or an antigen-binding fragment thereof.

In some cases, the antigen-binding protein or the MAGE-A4-specific CAR discussed above or herein comprises three heavy chain CDRs (HCDR1, HCDR2, and HCDR3) contained within a HCVR comprising the amino acid sequence set forth in SEQ ID NO: 2 or SEQ ID NO: 83. In some cases, HCDR1 comprises the amino acid sequence set forth in SEQ ID NO: 4, HCDR2 comprises the amino acid sequence set forth in SEQ ID NO: 6, and HCDR3 comprises the amino acid sequence set forth in SEQ ID NO: 8. In some cases, the HCVR comprises the amino acid sequence set forth in SEQ ID NO: 2. In some cases, the HCVR comprises the amino acid sequence set forth in SEQ ID NO: 83. In some cases, the antigen-binding protein or the MAGE-A4-specific CAR comprises three heavy chain CDRs (HCDR1, HCDR2, and HCDR3) contained within a HCVR comprising the amino acid sequence set forth in SEQ ID NO: 107. In some cases, HCDR1 comprises the amino acid sequence set forth in SEQ ID NO: 109, HCDR2 comprises the amino acid sequence set forth in SEQ ID NO: 111, and HCDR3 comprises the amino acid sequence set forth in SEQ ID NO: 113. In some cases, the HCVR comprises the amino acid sequence set forth in SEQ ID NO: 107.

In some cases, the antigen-binding protein or the MAGE-A4-specific CAR discussed above or herein comprises three light chain CDRs (LCDR1, LCDR2, and LCDR3) contained within a LCVR comprising the amino acid sequence set forth in SEQ ID NO: 10 or SEQ ID NO: 115. In some cases, LCDR1 comprises the amino acid sequence set forth in SEQ ID NO: 12, LCDR2 comprises the amino acid sequence set forth in SEQ ID NO: 14, and LCDR3 comprises the amino acid sequence set forth in SEQ ID NO: 16. In some cases, the LCVR comprises the amino acid sequence set forth in SEQ ID NO: 10. In some cases, LCDR1 comprises the amino acid sequence set forth in SEQ ID NO: 117, LCDR2 comprises the amino acid sequence set forth in SEQ ID NO: 14, and LCDR3 comprises the amino acid sequence set forth in SEQ ID NO: 119. In some cases, the LCVR comprises the amino acid sequence set forth in SEQ ID NO: 115.

In some cases, the antigen-binding protein or the MAGE-A4-specific CAR discussed above or herein comprises a HCVR that comprises the amino acid sequence set forth in SEQ ID NO: 2 and a LCVR that comprises the amino acid sequence set forth in SEQ ID NO: 10. In some cases, the antigen-binding protein or the MAGE-A4-specific CAR discussed above or herein comprises a HCVR that comprises the amino acid sequence set forth in SEQ ID NO: 83 and a LCVR that comprises the amino acid sequence set forth in SEQ ID NO: 10. In some cases, the antigen-binding protein or the MAGE-A4-specific CAR discussed above or herein comprises a HCVR that comprises the amino acid sequence set forth in SEQ ID NO: 107 and a LCVR that comprises the amino acid sequence set forth in SEQ ID NO: 115.

In various embodiments, the MAGE-A4-specific CAR comprises the amino acid sequence of SEQ ID NO: 22. In various embodiments, the MAGE-A4-specific CAR comprises the amino acid sequence of SEQ ID NO: 105. In various embodiments, the MAGE-A4-specific CAR comprises the amino acid sequence of SEQ ID NO: 120. In various embodiments, the MAGE-A4-specific CAR comprises the amino acid sequence of SEQ ID NO: 121.

In various embodiments, the antigen-binding protein or the MAGE-A4-specific CAR discussed above or herein specifically binds to one or more amino acids at positions 286-294 of SEQ ID NO: 32. In various embodiments, the antigen-binding protein or the MAGE-A4-specific CAR discussed above or herein interacts with one or more amino acids of the HLA. In some cases, the HLA is HLA-A2.

In one aspect, the present disclosure provides an isolated nucleic acid molecule encoding the antigen-binding protein or the MAGE-A4-specific CAR discussed above or herein. In some cases, the isolated nucleic acid molecule comprises a nucleotide sequence of SEQ ID NO: 21. In some cases, the isolated nucleic acid molecule comprises a nucleotide sequence of SEQ ID NO: 104.

In one aspect, the present disclosure provides a vector comprising the nucleic acid molecule discussed above or herein. In some cases, the vector is a DNA vector, an RNA vector, a plasmid, a lentivirus vector, an adenovirus vector, or a retroviral vector. In some embodiments, the vector is a lentivirus vector.

In one aspect, the present disclosure provides a cell comprising the nucleic acid molecule(s) discussed above or herein. In some cases, the cell is a human T cell.

In one aspect, the present disclosure provides an engineered cell comprising an antigen-binding protein or a MAGE-A4-specific CAR discussed above or herein. In some cases, the engineered cell is an immune cell. In some cases, the immune cell is an immune effector cell. In some cases, the immune effector cell is a T lymphocyte. In some cases, the T lymphocyte is an inflammatory T lymphocyte, a cytotoxic T lymphocyte, a regulatory T lymphocyte, or a helper T lymphocyte. In some cases, the engineered cell is a CD8+ cytotoxic T lymphocyte. In various embodiments, the engineered cell is for use in the treatment of a MAGE-A4-expressing cancer. In some cases, the MAGE-A4-expressing cancer is multiple myeloma. In some cases, the MAGE-A4-expressing cancer is melanoma.

In one aspect, the present disclosure provides an engineered human T cell comprising a chimeric antigen receptor comprising, from N-terminus to C-terminus: (a) an extracellular ligand-binding domain comprising an anti-MAGE-A4 single chain variable fragment (scFv) domain comprising a light chain variable region (LCVR) and a heavy chain variable region (HCVR); (b) a hinge; (c) a transmembrane domain; and (d) a cytoplasmic domain comprising a 4-1BB costimulatory domain or a CD28 costimulatory domain and a CD3zeta signaling domain, wherein the LCVR comprises complementarity determining regions (CDRs) of a LCVR comprising the amino acid sequence of SEQ ID NO: 10 or SEQ ID NO: 115, and CDRs of a HCVR comprising an amino acid sequence of SEQ ID NO: 2, SEQ ID NO: 83 or SEQ ID NO: 107.

In various embodiments of the engineered human T cell, the anti-MAGE-A4 scFv specifically binds to one or more amino acid residues of positions 286-294 of SEQ ID NO: 32. In some cases, the scFv domain comprises a HCVR/LCVR amino acid sequence pair comprising the amino acid sequences of SEQ ID NOs: 2/10. In some cases, the scFv domain comprises a HCVR/LCVR amino acid sequence pair comprising the amino acid sequences of SEQ ID NOs: 2/83. In some cases, the scFv domain comprises a HCVR/LCVR amino acid sequence pair comprising the amino acid sequences of SEQ ID NOs: 107/115. In some embodiments, the HCVR comprises three heavy chain CDRs (HCDR1, HCDR2, and HCDR3) and the LCVR comprises three light chain CDRs (LCDR1, LCDR2, and LCDR3), wherein HCDR1 comprises the amino acid sequence set forth in SEQ ID NO: 4, HCDR2 comprises the amino acid sequence set forth in SEQ ID NO: 6, HCDR3 comprises the amino acid sequence set forth in SEQ ID NO: 8, LCDR1 comprises the amino acid sequence set forth in SEQ ID NO: 12, LCDR2 comprises the amino acid sequence set forth in SEQ ID NO: 14, and LCDR3 comprises the amino acid sequence set forth in SEQ ID NO: 16. In some cases, the HCVR comprises three heavy chain CDRs (HCDR1, HCDR2, and HCDR3) and the LCVR comprises three light chain CDRs (LCDR1, LCDR2, and LCDR3), wherein HCDR1 comprises the amino acid sequence set forth in SEQ ID NO: 109, HCDR2 comprises the amino acid sequence set forth in SEQ ID NO: 111, HCDR3 comprises the amino acid sequence set forth in SEQ ID NO: 113, LCDR1 comprises the amino acid sequence set forth in SEQ ID NO: 117, LCDR2 comprises the amino acid sequence set forth in SEQ ID NO: 14, and LCDR3 comprises the amino acid sequence set forth in SEQ ID NO: 119. In some cases, the hinge comprises the amino acid sequence of SEQ ID NO: 27. In some cases, the transmembrane domain comprises the amino acid sequence of SEQ ID NO: 28. In some cases, the 4-1BB costimulatory domain comprises the amino acid sequence of SEQ ID NO: 29. In some cases, the hinge comprises the amino acid sequence of SEQ ID NO: 34. In some cases, the transmembrane domain comprises the amino acid sequence of SEQ ID NO: 36. In some cases, the CD28 costimulatory domain comprises the amino acid sequence of SEQ ID NO: 38. In some cases, the CD3zeta signaling domain comprises the amino acid sequence of SEQ ID NO: 30.

In various embodiments, the engineered human T cell comprises a chimeric antigen receptor comprising the amino acid sequence of SEQ ID NO: 22. In various embodiments, the engineered human T cell comprises a chimeric antigen receptor comprising the amino acid sequence of SEQ ID NO: 105. In various embodiments, the engineered human T cell comprises a chimeric antigen receptor comprising the amino acid sequence of SEQ ID NO: 120. In various embodiments, the engineered human T cell comprises a chimeric antigen receptor comprising the amino acid sequence of SEQ ID NO: 121.

In one aspect, the present disclosure provides a pharmaceutical composition comprising a genetically-modified human T cell and a pharmaceutically acceptable carrier, wherein the genetically-modified human T cell comprises an antigen-binding protein or a MAGE-A4-specific CAR discussed above or herein. In some cases, the pharmaceutical composition comprises an engineered cell as discussed above or herein and a pharmaceutically acceptable carrier. In some cases, the pharmaceutical composition comprises an engineered human T cell as discussed above or herein and a pharmaceutically acceptable carrier. In some embodiments, the pharmaceutical composition is for use in the treatment of a MAGE-A4-expressing cancer. In some cases, the MAGE-A4-expressing cancer is multiple myeloma. In some cases, the MAGE-A4-expressing cancer is melanoma.

In one aspect, the present disclosure provides use of the antigen-binding protein or the MAGE-A4-specific CAR, the nucleic acid molecule, the vector, the cell, the engineered cell, or the engineered human T cell, as discussed above or herein, in the manufacture of a medicament for the treatment of a MAGE-A4-expressing cancer. In some cases, the MAGE-A4-expressing cancer is multiple myeloma. In some cases, the MAGE-A4-expressing cancer is melanoma.

In one aspect, the present disclosure provides a method of enhancing T lymphocyte activity in a subject comprising, introducing into the subject a T lymphocyte comprising an antigen-binding protein or a MAGE-A4-specific CAR discussed above or herein.

In one aspect, the present disclosure provides a method for treating a subject suffering from cancer, comprising introducing into the subject a therapeutically effective amount of a T lymphocyte comprising an antigen-binding protein or a MAGE-A4-specific CAR discussed above or herein.

In one aspect, the present disclosure provides a method for stimulating a T cell-mediated immune response to a target cell population or tissue in a subject comprising, administering to the subject an effective amount of a cell genetically modified to express an antigen-binding protein or a MAGE-A4-specific CAR discussed above or herein.

In one aspect, the present disclosure provides a method of providing anti-tumor immunity in a subject, the method comprising administering to the subject an effective amount of a cell genetically modified to express an antigen-binding protein or a MAGE-A4-specific CAR discussed above or herein.

In any of the various methods discussed above or herein, the subject may be a human. In some embodiments, the subject has multiple myeloma, synovial sarcoma, esophageal cancer, head and neck cancer, lung cancer, bladder cancer, ovarian cancer, uterine cancer, stomach cancer, cervical cancer, breast cancer, or melanoma. In some embodiments, the subject has multiple myeloma.

In one aspect, the present disclosure provides a method of engineering a population of cells to express an antigen-binding protein or a MAGE-A4-specific CAR discussed above or herein, comprising: (a) introducing into a population of immune cells nucleic acid molecules encoding an antigen-binding protein or a MAGE-A4-specific CAR discussed above or herein; (b) culturing the population of immune cells under conditions to express said nucleic acid molecules; and (c) isolating the immune cells expressing said MAGE-A4-specific antigen-binding protein at the cells' surface. In some cases, the method further comprises obtaining the population of immune cells from a subject prior to introducing the nucleic acid molecule.

In one aspect, the present disclosure provides a method of treating a MAGE-A4-expressing cancer in a subject, comprising: (a) engineering a population of cells as discussed above or herein; and (b) reintroducing the population of cells expressing the chimeric antigen receptor into the subject. In some cases, the MAGE-A4-expressing cancer is multiple myeloma.

In one aspect, the present disclosure provides an isolated antigen-binding protein, wherein the antigen-binding protein comprises a first antigen-binding domain that binds specifically to an HLA-bound Melanoma-Associated Antigen A4 (MAGE-A4) and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (A1-HCVR) and three light chain CDRs (A1-LCDR1, A1-LCDR2 and A1-LCDR3) contained in a light chain variable region (A1-LCVR), and the second antigen-binding domain comprises three heavy chain CDRs (A2-HCDR1, A2-HCDR2 and A2-HCDR3) contained in a heavy chain variable region (A2-HCVR) and three light chain CDRs (A2-LCDR1, A2-LCDR2 and A2-LCDR3) contained in a light chain variable region (A2-LCVR), wherein A1-HCVR comprises the amino acid sequence of SEQ ID NO: 2, A1-LCVR comprises the amino acid sequence of SEQ ID NO: 10, A2-HCVR comprises the amino acid sequence of SEQ ID NO: 55, and A2-LCVR comprises the amino acid sequence of SEQ ID NO: 10.

In one aspect, the present disclosure provides an isolated antigen-binding protein, wherein the antigen-binding protein comprises a first antigen-binding domain that binds specifically to an HLA-bound Melanoma-Associated Antigen A4 (MAGE-A4) and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (A1-HCVR) and three light chain CDRs (A1-LCDR1, A1-LCDR2 and A1-LCDR3) contained in a light chain variable region (A1-LCVR), and the second antigen-binding domain comprises three heavy chain CDRs (A2-HCDR1, A2-HCDR2 and A2-HCDR3) contained in a heavy chain variable region (A2-HCVR) and three light chain CDRs (A2-LCDR1, A2-LCDR2 and A2-LCDR3) contained in a light chain variable region (A2-LCVR), wherein A1-HCVR comprises the amino acid sequence of SEQ ID NO: 2, A1-LCVR comprises the amino acid sequence of SEQ ID NO: 10, A2-HCVR comprises the amino acid sequence of SEQ ID NO: 73, and A2-LCVR comprises the amino acid sequence of SEQ ID NO: 10.

In various embodiments of the isolated antigen-binding protein discussed above or herein, the antigen-binding protein interacts with amino acids 286-294, or a portion thereof, of SEQ ID NO: 32. In some cases, isolated antigen-binding protein is a CAR. In some cases, the isolated antigen-binding protein is a bispecific antibody. In some embodiments, the isolated antigen-binding protein interacts with CD3. In some cases, the isolated antigen binding protein comprises a heavy chain variable region (HCVR) comprising an amino acid sequence having at least 95% sequence identity to SEQ ID NO: 2, or SEQ ID NO: 83. In some cases, the isolated antigen binding protein comprises a heavy chain variable region (HCVR) comprising an amino acid sequence having at least 95% sequence identity to SEQ ID NO: 55. In some cases, the isolated antigen binding protein comprises a heavy chain variable region (HCVR) comprising an amino acid sequence having at least 95% sequence identity to SEQ ID NO: 73.

In one aspect, the present disclosure provides an isolated antigen-binding protein that binds to the same epitope as an antigen-binding protein or a MAGE-A4-specific CAR discussed above or herein. In one aspect, the present disclosure provides an isolated antigen-binding protein that competes for binding with an antigen-binding protein or a MAGE-A4-specific CAR discussed above or herein. In some cases, the isolated antigen-binding protein is a CAR. In some cases, the isolated antigen-binding protein is a bispecific antibody. In some cases, the isolated antigen-binding protein interacts with amino acids 286-294, or a portion thereof, of SEQ ID NO: 32. In some cases, the isolated antigen-binding protein interacts with CD3. In some embodiments, the isolated antigen binding protein comprises a heavy chain variable region (HCVR) comprising three heavy chain CDRs, HCDR1, HCDR2 and HCDR3, comprising the amino acid sequences of SEQ ID NOs: 4, 6 and 8, respectively. In some embodiments, the isolated antigen binding protein comprises a HCVR corresponding to another arm of the bispecific antibody comprising three heavy chain CDRs, HCDR1, HCDR2 and HCDR3, comprising the amino acid sequences of SEQ ID NOs: 57, 59 and 61, respectively. In some embodiments, the isolated antigen binding protein comprises a HCVR corresponding to another arm of the bispecific antibody comprising three heavy chain CDRs comprising the amino acid sequences of SEQ ID NOs: 75, 77 and 79, respectively. In some cases, the isolated antigen binding protein comprises a light chain variable region (LCVR) comprising an amino acid sequence having at least 95% sequence identity to SEQ ID NO: 10 and/or an LCVR comprising an amino acid sequence having at least 95% sequence identity to SEQ ID NO: 63.

In one aspect, the present disclosure provides an isolated recombinant antibody or antigen-binding fragment thereof that specifically binds to a Melanoma-Associated Antigen A4 (MAGE-A4) polypeptide, wherein the antibody has one or more of the following characteristics: (a) binds to the MAGE-A4 polypeptide with an EC50 of less than about 2×10⁻⁹ M; (b) demonstrates an ability to reduce tumor cell viability as compared to isolated recombinant antibodies that do not specifically bind the MAGE-A4 polypeptide; and/or (c) comprises (i) three heavy chain complementarity determining regions (CDRs) (HCDR1, HCDR2, and HCDR3) contained within a heavy chain variable region (HCVR) comprising an amino acid sequence having at least about 90% sequence identity to an HCVR set forth in Table 1; and (ii) three light chain CDRs (LCDR1, LCDR2, and LCDR3) contained within a light chain variable region (LCVR) comprising the amino acid sequence having at least about 90% sequence identity to an LCVR set forth in Table 1.

In some embodiments of the isolated recombinant antibody or antigen-binding fragment thereof that specifically binds to a MAGE-A4 polypeptide, the MAGE-A4 polypeptide is an HLA-A2 bound MAGE-A4 polypeptide. In some cases, the isolated antibody or antigen-binding fragment thereof comprises an HCVR having an amino acid sequence of SEQ ID NO: 2, SEQ ID NO: 83, or SEQ ID NO: 107. In some cases, the isolated antibody or antigen-binding fragment thereof comprises an LCVR having an amino acid sequence of SEQ ID NO: 10 or SEQ ID NO: 115. In some cases, the isolated antibody or antigen-binding fragment thereof comprises an HCVR/LCVR amino acid sequence pair of SEQ ID NOs: 2/10, or SEQ ID NOs: 83/10, or SEQ ID NOs: 107/115.

In one aspect, the present disclosure provides an isolated antibody or antigen-binding fragment thereof comprising: (a) an HCDR1 domain having an amino acid sequence of SEQ ID NO: 4; (b) an HCDR2 domain having an amino acid sequence of SEQ ID NO: 6; (c) an HCDR3 domain having an amino acid sequence of SEQ ID NO: 8; (d) an LCDR1 domain having an amino acid sequence of SEQ ID NO: 12; (e) an LCDR2 domain having an amino acid sequence of SEQ ID NO: 14; and (f) an LCDR3 domain having an amino acid sequence of SEQ ID NO: 16.

In one aspect, the present disclosure provides an isolated antibody or antigen-binding fragment thereof comprising: (a) an HCDR1 domain having an amino acid sequence of SEQ ID NO: 109; (b) an HCDR2 domain having an amino acid sequence of SEQ ID NO: 111; (c) an HCDR3 domain having an amino acid sequence of SEQ ID NO: 113; (d) an LCDR1 domain having an amino acid sequence of SEQ ID NO: 117; (e) an LCDR2 domain having an amino acid sequence of SEQ ID NO: 14; and (f) an LCDR3 domain having an amino acid sequence of SEQ ID NO: 119.

In various embodiments of the isolated antibody or antigen-binding fragment thereof discussed above or herein, the isolated antibody or antigen-binding fragment thereof is an IgG1 antibody or an IgG4 antibody. In some cases, the isolated antibody or antigen-binding fragment thereof is a bispecific antibody.

In one aspect, the present disclosure provides an isolated recombinant antibody or antigen-binding fragment thereof comprising a first antigen-binding domain that specifically binds to a Melanoma-Associated Antigen A4 (MAGE-A4) polypeptide and a second antigen-binding domain that specifically binds to a CD3 polypeptide, wherein: (a) the first antigen-binding domain (A1) that binds MAGE-A4 comprises three heavy chain complementarity determining regions (A1-HCDR1, A1-HCDR2, and A1-HCDR3) and three light chain complementarity determining regions (A1-LCDR1, A1-LCDR2, and A1-LCDR3), wherein: A1-HCDR1 comprises the amino acid sequence of SEQ ID NO: 4 or SEQ ID NO: 109; A1-HCDR2 comprises the amino acid sequence of SEQ ID NO: 6 or SEQ ID NO: 111; A1-HCDR3 comprises the amino acid sequence of SEQ ID NO: 8 or SEQ ID NO: 113; A1-LCDR1 comprises the amino acid sequence of SEQ ID NO: 12, SEQ ID NO: 65, or SEQ ID NO: 117; A1-LCDR2 comprises the amino acid sequence of SEQ ID NO: 14; A1-LCDR3 comprises the amino acid sequence of SEQ ID NO: 16, SEQ ID NO: 67, or SEQ ID NO: 119; and (b) the second antigen-binding domain (A2) that binds CD3 comprises three heavy chain complementarity determining regions (A2-HCDR1, A2-HCDR2, and A2-HCDR3) and three light chain complementarity determining regions (A2-LCDR1, A2-LCDR2, and A2-LCDR3), wherein: A2-HCDR1 comprises the amino acid sequence of SEQ ID NO: 57 or SEQ ID NO: 75; A2-HCDR2 comprises the amino acid sequence of SEQ ID NO: 59 or SEQ ID NO: 77; A2-HCDR3 comprises the amino acid sequence of SEQ ID NO: 61 or SEQ ID NO: 79; A2-LCDR1 comprises the amino acid sequence of SEQ ID NO: 12, SEQ ID NO: 65, or SEQ ID NO: 117; A2-LCDR2 comprises the amino acid sequence of SEQ ID NO: 14; A2-LCDR3 comprises the amino acid sequence of SEQ ID NO: 16, SEQ ID NO: 67, or SEQ ID NO: 119.

In one aspect, the present disclosure provides a pharmaceutical composition comprising the isolated antibody or antigen-binding fragment thereof as discussed above or herein, and a pharmaceutically acceptable carrier or diluent. In some cases, the pharmaceutical composition further comprises a second therapeutic agent. In some cases, the second therapeutic agent is selected from the group consisting of: an anti-tumor agent, steroids, and targeted therapies.

In one aspect, the present disclosure provides a polynucleotide molecule comprising a polynucleotide sequence that encodes one or more HCVRs and/or one or more LCVRs of an antibody as discussed above or herein, and a vector comprising the polynucleotide, and a cell comprising the vector.

In one aspect, the present disclosure provides a method of treating a MAGE-A4 expressing cancer, the method comprising administering an antibody or antigen-binding fragment as discussed above or herein, or the pharmaceutical composition as discussed above or herein, to a subject. In some cases, the pharmaceutical composition is administered in combination with a second therapeutic agent. In some cases, the second therapeutic agent is selected from the group consisting of: an anti-tumor agent, steroids, and targeted therapies.

Other embodiments will become apparent from a review of the ensuing detailed description.

### BRIEF DESCRIPTION OF THE DRAWINGS

FIG. 1 illustrates an exemplary nucleotide construct for expressing a chimeric antigen receptor (CAR) construct. The exemplary nucleotide construct comprises an anti-MAGE-A4 VL-linker-VH scFv, a human CD8 hinge and transmembrane domain, a 4-1BB co-stimulatory domain, a CD3zeta signaling domain, and optionally an IRES:eGFP sequence for tracking CAR-transduced cells.

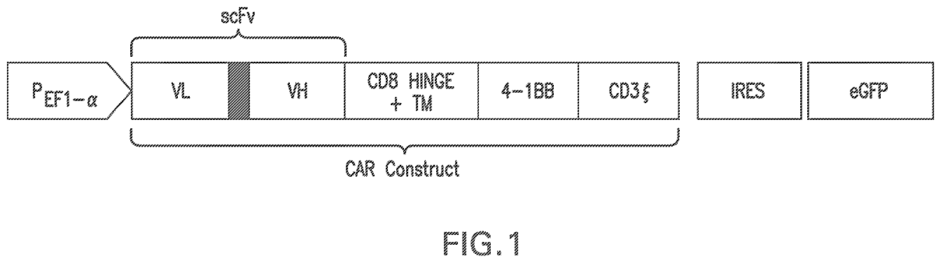

FIG. 2 illustrates an exemplary bispecific antibody of the present disclosure. The exemplary bispecific antibody is comprised of one HCVR capable of interacting with MAGE-A4 (anti-MAGE-A4 arm), and another HCVR capable of interacting with CD3 (anti-CD3 arm). In a representative example, the LCVRs are common (e.g., both corresponding to an anti-CD3 antibody or antibody light chain known to be promiscous or to pair effectively with a variety of heavy chain arms).

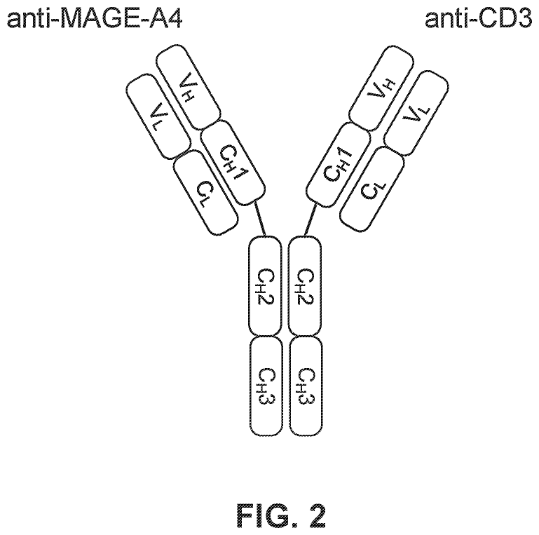

### DETAILED DESCRIPTION

It is to be understood that the inventions described herein are not limited to particular methods and experimental conditions described, and accordingly, methods and conditions may vary. It is also to be understood that the terminology used herein is for the purpose of describing particular embodiments only, and is not intended to be limiting, since the scope of the present inventions will be limited only by the appended claims. Any embodiments or features of embodiments can be combined with one another, and such combinations are expressly encompassed within the scope of the present invention. Any specific value discussed above or herein may be combined with another related value discussed above or herein to recite a range with the values representing the upper and lower ends of the range, and such ranges are encompassed within the scope of the present disclosure.

Unless defined otherwise, all technical and scientific terms used herein have the same meaning as commonly understood by one of ordinary skill in the art to which the inventions belong. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present inventions, the preferred methods and materials are now described. All patents, applications and non-patent publications mentioned in this specification are incorporated herein by reference in their entireties.

### Definitions

The term “MAGE-A4,” as used herein, refers to Melanoma-Associated Antigen A4. MAGE-A4 is an intracellular protein expressed by a variety of different tumor cells. As used herein, “MAGE-A4” refers to the human MAGE-A4 protein unless specified as being from a non-human species (e.g., “mouse MAGE-A4,” “monkey MAGE-A4,” etc.). The human MAGE-A4 protein has the amino acid sequence shown in SEQ ID NO: 32 and the polynucleic acid sequence of SEQ ID NO: 31. Reference to particular regions of a MAGE-A4 polypeptide (e.g., MAGE-A4 286-294) are with respect to SEQ ID NO: 32. As used herein, the terms “MAGE-A4 286-294,” “MAGE-A4 (286-294),” and “MAGEA4286-294” may be used interchangeably. The polypeptide sequence of MAGE-A4 (286-294) (KVLEHVVRV) is given as SEQ ID NO: 33.

As used herein, “an antibody that binds MAGE-A4” or an “anti-MAGE-A4 antibody” includes antibodies and antigen-binding fragments thereof that specifically recognize at least MAGE-A4. In some embodiments, an antibody that binds MAGE-A4 interacts with amino acids 286-294 of MAGE-A4. As herein disclosed, an “antibody that binds MAGE-A4” or an “anti-MAGE-A4 antibody” may additionally be capable of specifically recognizing other MAGE-A4-related peptides (e.g., MAGE-A4-related peptides predicted to form complexes with HLA-A2). Moreover, an antibody that binds MAGE-A4 may further be able to bind one or more additional ligands, e.g., if formatted as a bispecific antibody. In certain embodiments, an anti-MAGE-A4 antibody may be formatted as a bispecific antibody that binds to both MAGE-A4 and CD3.

The terms “ligand-binding domain” and “antigen-binding domain” are used interchangeably herein, and refer to that portion of a chimeric antigen receptor or a corresponding antibody that binds specifically to a predetermined antigen (e.g., MAGE-A4). References to a “corresponding antibody” refer to the antibody from which the CDRs or variable regions (heavy chain variable region (abbreviated HCVR or VH) and light chain variable region (abbreviated LCVR or VL)) used in a chimeric antigen receptor or bispecific antibody are derived. For example, chimeric antigen receptor constructs discussed in the Examples include scFvs with variable regions derived from an anti-MAGE-A4 antibody. This anti-MAGE-A4 antibody is the “corresponding antibody” to the respective chimeric antigen receptor.

The term “antibody”, as used herein, which includes a “bispecific antibody”, means any antigen-binding molecule or molecular complex comprising at least one complementarity determining region (CDR) that specifically binds to or interacts with a particular antigen (e.g., MAGE-A4). In some embodiments, an antibody can bind to or interact with an MHC-bound polypeptide, such as an HLA-bound polypeptide. In the context of the present disclosure, an antibody can, in some embodiments, bind to an HLA-A2-bound polypeptide such as a MAGE-A4 polypeptide (e.g., MAGE-A4 286-294) presented by HLA-A2. The term “antibody”, which includes a “bispecific antibody”, includes immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody”. The term “antibody” also includes immunoglobulin molecules consisting of four polypeptide chains, two heavy (H) chains and two light (L) chains inter-connected by disulfide bonds. Each heavy chain (abbreviated herein as HC) comprises a heavy chain variable region (abbreviated herein as HCVR or V_{H}) and a heavy chain constant region. The heavy chain constant region comprises three domains, C_{H}1, C_{H}2 and C_{H}3. Each light chain (abbreviated herein as LC) comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the present disclosure, the FRs of the anti-MAGE-A4 antibody (or antigen-binding portion thereof) may be identical to the human germline sequences, or may be naturally or artificially modified. An amino acid consensus sequence may be defined based on a side-by-side analysis of two or more CDRs.

In certain embodiments of the invention, the antibodies are bispecific antibodies. For example, in certain embodiments, an anti-MAGE-A4 antibody (e.g., mAbM31339N2) is reformatted into bispecific antibodies (e.g., anti-MAGE-A4×anti-CD3) utilizing either a high affinity (7195P) CD3 arm to generate bsb6054, or a medium affinity (7221G) CD3 arm to generate bsAb6043.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. As used herein, an “antigen-binding portion” or an “antigen-binding fragment” refers to one or more fragments of an antibody that retains the ability to specifically bind to MAGE-A4 (or MAGE-A4-related peptide) and/or CD3. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} Or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In certain embodiments, the anti-MAGE-A4 antibodies from which antigen-binding fragments are derived are human antibodies. The term “human antibody”, as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

In various embodiments, the anti-MAGE-A4×anti-CD3 bispecific antibodies (e.g., bsAb 6054 and/or bsAb 6043) of the present disclosure are human antibodies. In various embodiments, the anti-MAGE-A4×anti-CD3 bispecific antibody is a human IgG antibody. In various embodiments, the anti-MAGE-A4×anti-CD3 bispecific antibody is a human antibody of isotype IgG1, IgG2, IgG3 or IgG4, or mixed isotype. In some embodiments, the anti-MAGE-A4×CD3 bispecific antibody is a human IgG1 antibody (i.e., the antibody comprises a human IgG1 heavy chain constant region attached, respectively, to the HCVR of each of the first antigen-binding domain and the second antigen-binding domain). In some embodiments, the anti-MAGE-A4×anti-CD3 bispecific antibody is a human IgG4 antibody (i.e., the antibody comprises a human IgG4 heavy chain constant region attached, respectively, to the HCVR of each of the first antigen-binding domain and the second antigen-binding domain. In any of the embodiments discussed above or herein, the anti-MAGE-A4×anti-CD3 bispecific antibody may comprise a human kappa light chain. In any of the embodiments discussed above or herein, the anti-MAGE-A4×anti-CD3 bispecific antibody may comprise a human lambda light chain.

In any embodiments, the bispecific antibody may include a modification in one or both heavy chains to facilitate purification of the bispecific antibody (i.e., the heterodimer) from homodimeric impurities. In some embodiments, the bispecific antibodies include first and second heavy chains (i.e., the heavy chain of the anti-MAGE-A4 binding arm, and the heavy chain of the anti-CD3 binding arm) that are identical (e.g., both of isotype IgG1 or IgG4) except for a modification in the CH3 domain of one or the other heavy chain that reduces binding of the bispecific antibody to Protein A as compared to an antibody lacking the modification. In some cases, the CH3 domain of the first heavy chain (e.g., of the anti-MAGE-A4 binding arm) binds Protein A and the CH3 domain of the second heavy chain (e.g., of the anti-CD3 binding arm) contains a mutation that reduces or abolishes Protein A binding. In some cases, the mutation is a H435R modification (by EU numbering; H95R by IMGT exon numbering). In some cases, the mutation is a H435R modification (by EU numbering; H95R by IMGT exon numbering) and a Y436F modification (by EU numbering; Y96F by IMGT). Further modifications that may be found within the second CH3 domain include: D356E, L358M, N384S, K392N, V397M, and V422I by EU (D16E, L18M, N44S, K52N, V57M, and V82I by IMGT) in the case of IgG1 CH3 domains; and Q355R, N384S, K392N, V397M, R409K, E419Q, and V422I by EU (Q15R, N44S, K52N, V57M, R69K, E79Q, and V82I by IMGT) in the case of IgG4 CH3 domains.

In any embodiments, the bispecific antibody may include a chimeric hinge. The term “chimeric hinge” is intended to include a chimeric protein comprising a first amino acid sequence derived from the hinge region of one Ig molecule and a second amino acid sequence derived from the hinge region of a different class or subclass of Ig molecule. For example, the chimeric hinge comprises, in an embodiment, a first amino acid sequence, or an “upper hinge” sequence, derived from a human IgG1 hinge region or human IgG4 hinge region, and a second amino acid sequence, or a “lower hinge” sequence, derived from a human IgG2 hinge region. In certain embodiments, the first or “upper hinge” sequence comprises amino acid residues from positions 216 to 227 according to EU numbering. In some embodiments, the second or “lower hinge” sequence comprises amino acid residues from positions 228 to 236 according to EU numbering.

The antibodies of the present disclosure, which include bispecific antibodies, those used to generate anti-MAGE-A4 antigen-binding fragments, and/or chimeric antigen receptors may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The present disclosure encompasses antibodies having one or more mutations in the hinge, CH2 or CH3 region which may be desirable, for example, in production, to improve the yield of the desired antibody form.

Antibodies as herein disclosed, which includes bispecific antibodies, may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. Thus, an isolated antibody also includes an antibody that is substantially free of other antibodies having different antigenic specificities (e.g., an isolated antibody that specifically binds human MAGE-A4, or human MAGE-A4 and human CD3, is substantially free of antibodies that specifically bind antigens other than human MAGE-A4, or human MAGE-A4 and human CD3). However, as herein disclosed an isolated antibody such as one that specifically binds human MAGE-A4 may in some examples additionally specifically bind to one or more other MAGE-A4-related proteins or peptides. According to certain embodiments, an isolated antibody may be substantially free of other cellular material and/or chemicals.

The term “specifically binds,” or the like, means that an antibody (which includes a bispecific antibody) or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds human MAGE-A4, or human MAGE-A4 and human CD3 may, however, have cross-reactivity to other antigens, such as MAGE-A4 and/or CD3 molecules from other species (orthologs). In the context of the present invention, mono-specific antibodies that bind to human MAGE-A4 as well as one or more additional antigens, including MAGE-A4-related peptides, are deemed to “specifically bind” human MAGE-A4. In the context of the present invention, multispecific (e.g., bispecific) antibodies that bind to human MAGE-A4 and human CD3 as well as one or more additional antigens are deemed to “specifically bind” human MAGE-A4 and human CD3.

Bispecific antibodies comprising an anti-MAGE-A4-specific binding domain and an anti-CD3-specific binding domain may be constructed using standard methodologies, wherein the anti-MAGE-A4 antigen binding domain and the anti-CD3 antigen binding domain each comprise different, distinct HCVRs paired with a common LCVR. In exemplified bispecific antibodies, the molecules were constructed utilizing a heavy chain from an anti-CD3 antibody, a heavy chain from an anti-MAGE-A4 antibody and a common light chain from the anti-MAGE-A4 antibody. In other instances, the bispecific antibodies may be constructed utilizing a heavy chain from an anti-CD3 antibody, a heavy chain from an anti-MAGE-A4 antibody and a light chain from an anti-CD3 antibody or an antibody light chain known to be promiscuous or pair effectively with a variety of heavy chain arms.

According to certain embodiments of the present invention, the anti-MAGE-A4×anti-CD3 bispecific antibody, or antigen-binding fragment thereof, comprises a first antigen-binding domain that specifically binds human MAGE-A4 and a second antigen-binding domain that specifically binds human CD3, in which the first antigen-binding domain comprises heavy chain CDRs A1-HCDR1, A1-HCDR2, and A1-HCDR3, respectively, comprising the amino acid sequences of SEQ ID NOs: 4, 6, and 8, and the second antigen-binding domain comprises heavy chain CDRs A2-HCDR1, A2-HCDR2, and A2-HCDR3, respectively, comprising the amino acid sequences of SEQ ID NOs: 57 or 75, 59 or 77, and 61 or 79, respectively. According to certain embodiments of the present invention, the anti-MAGE-A4×anti-CD3 bispecific antibody, or antigen-binding fragment thereof, comprises common (to both the first and second antigen-binding domains) light chain complementarity determining regions LCDR1-LCDR2-LCDR3, respectively. In one example, the common light chain complementary determining regions comprise the amino acid sequences of SEQ ID NOs: 12, 14, and 16. In other examples, the common light chain complementary determining regions comprise the amino acid sequences of SEQ ID NOs: 65, 14, and 67.

In certain embodiments, the anti-MAGE-A4×anti-CD3 bispecific antibody, or antigen-binding fragment thereof, comprises a first antigen-binding domain that specifically binds human MAGE-A4 and a second antigen-binding domain that specifically binds human CD3, in which the first antigen-binding domain comprises a heavy chain variable region (HCVR) comprising the amino acid sequence of SEQ ID NO: 2, SEQ ID NO: 83 or SEQ ID NO: 107, and the second antigen-binding domain comprises a HCVR comprising the amino acid sequence of SEQ ID NO: 55, or SEQ ID NO: 73. In certain embodiments, the anti-MAGE-A4×anti-CD3 bispecific antibody, or antigen-binding fragment thereof, comprises a common light chain variable region (LCVR) comprising the amino acid sequence of SEQ ID NO: 10, SEQ ID NO: 63, or SEQ ID NO: 115. In certain embodiments, the anti-MAGE-A4×anti-CD3 bispecific antibody, or antigen-binding fragment thereof, comprises a first antigen-binding domain comprising a HCVR/LCVR amino acid sequence pair comprising the amino acid sequences of SEQ ID NOs: 2/10, and a second antigen-binding domain comprising a HCVR/LCVR amino acid sequence pair comprising the amino acid sequences of SEQ ID NOs: 55/10, or comprising the amino acid sequences of SEQ ID NOs: 73/10. In certain embodiments, the anti-MAGE-A4×anti-CD3 bispecific antibody, or antigen-binding fragment thereof, comprises a first antigen-binding domain comprising a HCVR/LCVR amino acid sequence pair comprising the amino acid sequences of SEQ ID NOs: 83/10, and a second antigen-binding domain comprising a HCVR/LCVR amino acid sequence pair comprising the amino acid sequences of SEQ ID NOs: 55/10, or comprising the amino acid sequences of SEQ ID NOs: 73/10. In certain embodiments, the anti-MAGE-A4×anti-CD3 bispecific antibody, or antigen-binding fragment thereof, comprises a first antigen-binding domain comprising a HCVR/LCVR amino acid sequence pair comprising the amino acid sequences of SEQ ID NOs: 107/115, and a second antigen-binding domain comprising a HCVR/LCVR amino acid sequence pair comprising the amino acid sequences of SEQ ID NOs: 55/115, or comprising the amino acid sequences of SEQ ID NOs: 73/115. In certain embodiments, the anti-MAGE-A4×anti-CD3 bispecific antibody, or antigen-binding fragment thereof, comprises a first antigen-binding domain comprising a HCVR/LCVR amino acid sequence pair comprising the amino acid sequences of SEQ ID NOs: 2/63, and a second antigen-binding domain comprising a HCVR/LCVR amino acid sequence pair comprising the amino acid sequences of SEQ ID NOs: 55/63, or comprising the amino acid sequences of SEQ ID NOs: 73/63. In certain embodiments, the anti-MAGE-A4×anti-CD3 bispecific antibody, or antigen-binding fragment thereof, comprises a first antigen-binding domain comprising a HCVR/LCVR amino acid sequence pair comprising the amino acid sequences of SEQ ID NOs: 83/63, and a second antigen-binding domain comprising a HCVR/LCVR amino acid sequence pair comprising the amino acid sequences of SEQ ID NOs: 55/63, or comprising the amino acid sequences of SEQ ID NOs: 73/63. In certain embodiments, the anti-MAGE-A4×anti-CD3 bispecific antibody, or antigen-binding fragment thereof, comprises a first antigen-binding domain comprising a HCVR/LCVR amino acid sequence pair comprising the amino acid sequences of SEQ ID NOs: 107/63, and a second antigen-binding domain comprising a HCVR/LCVR amino acid sequence pair comprising the amino acid sequences of SEQ ID NOs: 55/63, or comprising the amino acid sequences of SEQ ID NOs: 73/63. In some embodiments, the anti-MAGE-A4×anti-CD3 bispecific antibody comprises the HCVR/LCVR sequence pairs noted above, and a human IgG1 heavy chain constant region. In some embodiments, the anti-MAGE-A4×anti-CD3 bispecific antibody comprises the HCVR/LCVR sequence pairs noted above, and a human IgG4 heavy chain constant region. In some embodiments, the anti-MAGE-A4×anti-CD3 bispecific antibody comprises the HCVR/LCVR sequence pairs noted above, and a human IgG heavy chain constant region. In some embodiments, the anti-MAGE-A4×anti-CD3 bispecific antibody comprises the HCVR/LCVR sequence pairs noted above, and a human IgG1 or IgG4 heavy chain constant region.

The anti-MAGE-A4 antibodies, or antigen binding fragments thereof, disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The anti-MAGE-A4 antibodies may comprise variants of any of the HCVR, LCVR, and/or CDR amino acid sequences disclosed herein having one or more conservative substitutions. For example, the anti-MAGE-A4 antibodies may have HCVR, LCVR, and/or CDR amino acid sequences with, e.g., 10 or fewer, 9 or fewer, 8 or fewer, 7 or fewer, 6 or fewer, 5 or fewer, 4 or fewer, 3 or fewer, 2 or fewer, or 1 conservative amino acid substitutions relative to any of the HCVR, LCVR, and/or CDR amino acid sequences set forth herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98%, 99%, 99.5%, or 99.9% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule. In some embodiments, the present disclosure provides a polypeptide comprising a sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% identical to the sequence of SEQ ID NO: 22 or SEQ ID NO: 105, or to a portion of SEQ ID NO: 22 or SEQ ID NO: 105 (e.g., an HCVR such as the sequence of SEQ ID NO: 2, or SEQ ID NO: 83, or an LCVR such as the sequence of SEQ ID NO: 10, or a framework region of a polypeptide sequence such as those found in SEQ ID NOs: 2, 10, 22, or 105). In some embodiments, the present disclosure provides a polynucleic acid encoding such a polypeptide. In some embodiments, the present disclosure provides a polynucleic acid comprising a sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% identical to the sequence of SEQ ID NO: 21 or SEQ ID NO: 104, or to a portion of SEQ ID NO: 21 or SEQ ID NO: 104 (e.g., an HCVR such as the sequence of SEQ ID NO: 1 or an LCVR such as the sequence of SEQ ID NO: 9, or a framework region of a polynucleotide sequence such as SEQ ID NOs: 1, 9, 21, or 104).

In some embodiments, the present disclosure provides a polypeptide comprising a sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% identical to the sequence of SEQ ID NO: 69, or a portion thereof, such as SEQ ID NO: 55; or to the sequence of SEQ ID NO: 71, or a portion thereof, such as SEQ ID NO: 63; or to the sequence of SEQ ID NO: 81, or a portion thereof, such as SEQ ID NO: 73. In some embodiments, the present disclosure provides a polynucleic acid comprising a sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or 100% identical to the sequence of SEQ ID NO: 68, or a portion thereof, such as SEQ ID NO: 54; or to the sequence of SEQ ID NO: 70, or a portion thereof, such as SEQ ID NO: 62; or to the sequence of SEQ ID NO: 80, or a portion thereof, such as SEQ ID NO: 72.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Sequences also can be compared using the Smith-Waterman homology search algorithm using an affine gap search with a gap open penalty of 12 and a gap extension penalty of 2, BLOSUM matrix of 62. Another preferred algorithm when comparing a sequence of the present disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

As used herein, the terms “nucleic acid” or “polynucleotide” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The term “chimeric antigen receptor” (CAR) refers to molecules that combine a binding domain against a component present on the target cell, for example an antibody-based specificity for a desired antigen (e.g., a tumor antigen, such as MAGE-A4) with a T cell receptor-activating intracellular domain to generate a chimeric protein that exhibits a specific anti-target cellular immune activity. Generally, CARs consist of an extracellular single chain antibody-binding domain (scFv) fused to the intracellular signaling domain of the T cell antigen receptor complex zeta chain, and have the ability, when expressed in T cells, to redirect antigen recognition based on the monoclonal antibody's specificity.

The term “HLA” refers to the human leukocyte antigen (HLA) system or complex, which is a gene complex encoding the major histocompatibility complex (MHC) proteins in humans. These cell-surface proteins are responsible for the regulation of the immune system in humans. HLAs corresponding to MHC class I (A, B, and C) present peptides from inside the cell. In the context of the present application, a peptide can be “HLA-bound” if it is bound to an HLA system or complex. In some embodiments, an HLA-bound peptide is present on the surface of a cell.

The term “HLA-A” refers to the group of human leukocyte antigens (HLA) that are coded for by the HLA-A locus. HLA-A is one of three major types of human MHC class I cell surface receptors. The receptor is a heterodimer, and is composed of a heavy a chain and smaller β chain. The α chain is encoded by a variant HLA-A gene, and the β chain (β2-microglobulin) is an invariant β2 microglobulin molecule.

The term “HLA-A2” is one particular class I major histocompatibility complex (MHC) allele group at the HLA-A locus; the α chain is encoded by the HLA-A*02 gene and the β chain is encoded by the β2-microglobulin or B2M locus.

The term “vector,” as used herein, includes, but is not limited to, a viral vector, a plasmid, an RNA vector or a linear or circular DNA or RNA molecule which may consists of chromosomal, non-chromosomal, semi-synthetic or synthetic nucleic acids. In some cases, the vectors are those capable of autonomous replication (episomal vector) and/or expression of nucleic acids to which they are linked (expression vectors). Large numbers of suitable vectors are known to those of skill in the art and are commercially available. Viral vectors include retrovirus, adenovirus, parvovirus (e.g., adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, and lentivirus.

A “costimulatory domain” or “costimulatory molecule” refers to the cognate binding partner on a T-cell that specifically binds with a costimulatory ligand, thereby mediating a costimulatory response by the cell, such as, but not limited to proliferation. Costimulatory molecules include, but are not limited to, an MHC class I molecule, BTLA and Toll ligand receptor. Examples of costimulatory molecules include CD27, CD28, CD8, 4-1BB (CD137) (SEQ ID NO: 29), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like. A costimulatory molecule is a cell surface molecule other than an antigen receptor or their ligands that is required for an efficient immune response.

A “costimulatory ligand” refers to a molecule on an antigen presenting cell that specifically binds a cognate costimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A costimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, an agonist or antibody that binds Toll ligand receptor and a ligand that specifically binds with B7-H3.

A “costimulatory signal” refers to a signal, which in combination with a primary signal, such as TCR/CD3 ligation, leads to T cell proliferation and/or upregulation or downregulation of key molecules.

The term “extracellular ligand-binding domain,” as used herein, refers to an oligo- or polypeptide that is capable of binding a ligand, e.g., a cell surface molecule. For example, the extracellular ligand-binding domain may be chosen to recognize a ligand that acts as a cell surface marker on target cells associated with a particular disease state (e.g., cancer). Examples of cell surface markers that may act as ligands include those associated with viral, bacterial and parasitic infections, autoimmune disease and cancer cells. An extracellular ligand-binding domain can comprise LCVR and HCVR regions (e.g., formatted as an scFv), optionally joined by a linker.

The term “subject” or “patient” as used herein includes all members of the animal kingdom including non-human primates and humans. In one embodiment, patients are humans with a cancer (e.g., multiple myeloma or melanoma).

A “signal transducing domain” or “signaling domain” of a CAR, as used herein, is responsible for intracellular signaling following the binding of an extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector function signal and directs the cell to perform a specialized function. Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Exemplary ITAMs include those derived from TCRzeta, FcRgamma, FcRbeta, FcRepsilon, CD3gamma, CD3delta, CD3epsilon, CD5, CD22, CD79a, CD79b and CD66d. In some embodiments, the signal transducing domain of the CAR can comprise the CD3zeta signaling domain (SEQ ID NO: 30).

### Chimeric Antigen Receptors (CARs)

Chimeric antigen receptors (CARs) can redirect T cell specificity toward antibody-recognized antigens on the surface of cells (e.g., cancer cells), whether those antigens are expressed on the cell surface or expressed intracellularly and presented by, for example, an HLA.

One aspect of the present disclosure includes a chimeric antigen receptor (CAR) which is specific for a MAGE-A4 antigen presented on the surface of cells such as tumor cells. This presentation can be by, for example, an HLA such as HLA-A2. In one embodiment of the present disclosure, a CAR as described herein comprises an extracellular target-specific binding domain, a transmembrane domain, an intracellular signaling domain (such as a signaling domain derived from CD3zeta or FcRgamma), and/or one or more co-stimulatory signaling domains derived from a co-stimulatory molecule, such as, but not limited to, 4-1BB. In one embodiment, the CAR includes a hinge or spacer region between the extracellular binding domain and the transmembrane domain, such as a CD8alpha hinge.

The binding domain or the extracellular domain of the CAR provides the CAR with the ability to bind to the target antigen of interest. A binding domain (e.g., a ligand-binding domain or antigen-binding domain) can be any protein, polypeptide, oligopeptide, or peptide that possesses the ability to specifically recognize and bind to a biological molecule (e.g., a cell surface receptor or tumor protein, or a component thereof). A binding domain includes any naturally occurring, synthetic, semi-synthetic, or recombinantly produced binding partner for a biological molecule of interest. For example, and as further described herein, a binding domain may be antibody light chain and heavy chain variable regions, or the light and heavy chain variable regions can be joined together in a single chain and in either orientation (e.g., VL-VH or VH-VL). A variety of assays are known for identifying binding domains of the present disclosure that specifically bind with a particular target, including Western blot, ELISA, flow cytometry, or surface plasmon resonance analysis (e.g., using BIACORE analysis). The target may be an antigen of clinical interest against which it would be desirable to trigger an effector immune response that results in tumor killing. In one embodiment, the target antigen of the binding domain of the chimeric antigen receptor is a MAGE-A4 protein on the surface of tumor cells (e.g., an HLA-presented MAGE-A4 protein such as an HLA-A2-presented MAGE-A4 protein).

Illustrative ligand-binding domains include antigen binding proteins, such as antigen binding fragments of an antibody, such as scFv, scTCR, extracellular domains of receptors, ligands for cell surface molecules/receptors, or receptor binding domains thereof, and tumor binding proteins. In certain embodiments, the antigen binding domains included in a CAR of the present disclosure can be a variable region (Fv), a CDR, a Fab, an scFv, a VH, a VL, a domain antibody variant (dAb), a camelid antibody (VHH), a fibronectin 3 domain variant, an ankyrin repeat variant and other antigen-specific binding domain derived from other protein scaffolds.

In one embodiment, the binding domain of the CAR is an anti-MAGE-A4 single chain antibody (scFv), and may be a murine, human or humanized scFv. Single chain antibodies may be cloned from the V region genes of a hybridoma specific for a desired target. A technique which can be used for cloning the variable region heavy chain (VH) and variable region light chain (VL) has been described, for example, in Orlandi et al., PNAS, 1989; 86: 3833-3837. Thus, in certain embodiments, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In certain embodiments, the CARs of the present disclosure may comprise a linker between the various domains, added for appropriate spacing and conformation of the molecule. For example, in one embodiment, there may be a linker between the binding domain VH or VL which may be between 1 and 20 amino acids long. In other embodiments, the linker between any of the domains of the chimeric antigen receptor may be between 1 and 15 or 15 amino acids long. In this regard, the linker may be 1, 2, 3, 4, 5, 6, 7, 8, 9, 10, 11, 12, 13, 14, 15, 16, 17, 18, 19 or 20 amino acids long. In further embodiments, the linker may be 21, 22, 23, 24, 25, 26, 27, 28, 29 or 30 amino acids long. Ranges including the numbers described herein are also included herein, e.g., a linker 10-30 amino acids long.

In certain embodiments, linkers suitable for use in the CAR described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure. Specific linkers include (G4S)n linkers, wherein n=1-3, as shown in SEQ ID NOs: 23-25. Another exemplary linker is provided as SEQ ID NO: 26. A linker can be present between the LCVR and HCVR regions of a CAR, between a variable region (such as an HCVR) and a hinge region (such as a CD8a hinge), or both. For example, the present disclosure provides a CAR comprising a (G4S)3 linker between an LCVR and an HCVR, and a (G4S)1 linker between an HCVR and a CD8α hinge.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6: 412-419). The hinge region in a CAR is generally between the transmembrane (TM) and the binding domain. In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered. In one embodiment, the hinge region comprises a CD8alpha hinge (SEQ ID NO: 27).

The “transmembrane” region or domain is the portion of the CAR that anchors the extracellular binding portion to the plasma membrane of the immune effector cell, and facilitates binding of the binding domain to the target antigen. The transmembrane domain may be a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154. In one embodiment, the transmembrane domain is the transmembrane domain of CD137. In some embodiments, the transmembrane domain comprises the amino acid sequence of SEQ ID NO: 28. In certain embodiments, the transmembrane domain is synthetic in which case it would comprise predominantly hydrophobic residues such as leucine and valine.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain,” and is typically derived from portions of the human CD3 or FcRy chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM-containing primary cytoplasmic signaling sequences that are of particular use in the present disclosure include those derived from TCRzeta, FcRgamma, FcRbeta, CD3gamma, CD3delta, CD3epsilon, CD5, CD22, CD79a, CD79b and CD66d. In one particular embodiment, the intracellular signaling domain of the anti-MAGE-A4 CARs described herein are derived from CD3zeta. In some embodiments, the signaling domain comprises the amino acid sequence of SEQ ID NO: 30.

As used herein, the term, “costimulatory signaling domain,” or “costimulatory domain”, refers to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD3zeta and 4-1BB, other costimulatory domains are contemplated for use with the CARs described herein. The inclusion of one or more co-stimulatory signaling domains may enhance the efficacy and expansion of T cells expressing CAR receptors. The intracellular signaling and costimulatory signaling domains may be linked in any order in tandem to the carboxyl terminus of the transmembrane domain. In some embodiments, the costimulatory domain comprises the amino acid sequence of SEQ ID NO: 29.

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy, 2009; 17: 1453-1464; Zhong et al., Molecular Therapy, 2010; 18: 413-420; Carpenito et al., PNAS, 2009; 106:3360-3365).

In various embodiments, the anti-MAGE-A4 CARs of the present disclosure comprise (a) an anti-MAGE-A4 scFv as a binding domain (e.g., an scFv having binding regions (e.g., CDRs or variable domains) from the anti-MAGE-A4 antibody identified in Table 1) (b) a hinge region derived from human CD8alpha, (c) a human CD8alpha transmembrane domain, and (d) a human T cell receptor CD3 zeta chain (CD3) intracellular signaling domain, and optionally one or more costimulatory signaling domains, e.g., 4-1BB. In one embodiment, the different protein domains are arranged from amino to carboxyl terminus in the following order: binding domain, hinge region and transmembrane domain. The intracellular signaling domain and optional co-stimulatory signaling domains are linked to the transmembrane carboxy terminus in any order in tandem to form a single chain chimeric polypeptide. In one embodiment, a nucleic acid construct encoding an anti-MAGE-A4 CAR is a chimeric nucleic acid molecule comprising a nucleic acid molecule comprising different coding sequences, for example, (5′ to 3′) the coding sequences of a human anti-MAGE-A4 scFv, a human CD8alpha-hinge, a human CD8alpha transmembrane domain and a CD3zeta intracellular signaling domain. In another embodiment, a nucleic acid construct encoding an anti-MAGE-A4 CAR is a chimeric nucleic acid molecule comprising a nucleic acid molecule comprising different coding sequences, for example, (5′ to 3′) the coding sequences of a human anti-MAGE-A4 scFv, a human CD8alpha-hinge, a human CD8alpha transmembrane domain, a 4-1BB co-stimulatory domain, and a CD3zeta co-stimulatory domain.

In certain embodiments, the polynucleotide encoding the CAR described herein is inserted into a vector. The vector is a vehicle into which a polynucleotide encoding a protein may be covalently inserted so as to bring about the expression of that protein and/or the cloning of the polynucleotide. Such vectors may also be referred to as “expression vectors”. The isolated polynucleotide may be inserted into a vector using any suitable methods known in the art, for example, without limitation, the vector may be digested using appropriate restriction enzymes and then may be ligated with the isolated polynucleotide having matching restriction ends. Expression vectors can have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra. An expression vector may comprise additional elements, for example, the expression vector may have two replication systems, thus allowing it to be maintained in two organisms, for example in human cells for expression and in a prokaryotic host for cloning and amplification.

The expression vector may have the necessary 5′ upstream and 3′ downstream regulatory elements such as promoter sequences such as CMV, PGK and EF1alpha. promoters, ribosome recognition and binding TATA box, and 3′ UTR AAUAAA transcription termination sequence for the efficient gene transcription and translation in its respective host cell. Other suitable promoters include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing chimeric antigen receptor. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector may have additional sequence such as 6×-histidine, c-Myc, and FLAG tags which are incorporated into the expressed CARs. Thus, the expression vector may be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector may also be engineered for replication and/or expression functionality (e.g., transcription and translation) in a particular cell type, cell location, or tissue type. Expression vectors may include a selectable marker for maintenance of the vector in the host or recipient cell.

In various embodiments, the vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontrch), pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the CARs disclosed herein can be ligated into such expression vectors for the expression of the chimeric protein in mammalian cells.

In certain embodiments, the nucleic acids encoding the CAR of the present disclosure are provided in a viral vector. A viral vector can be that derived from retrovirus, lentivirus, or foamy virus. As used herein, the term, “viral vector,” refers to a nucleic acid vector construct that includes at least one element of viral origin and has the capacity to be packaged into a viral vector particle. The viral vector can contain the coding sequence for the various chimeric proteins described herein in place of nonessential viral genes. The vector and/or particle can be utilized for the purpose of transferring DNA, RNA or other nucleic acids into cells either in vitro or in vivo. Numerous forms of viral vectors are known in the art.

In certain embodiments, the viral vector containing the coding sequence for a CAR described herein is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

The retroviral vectors for use herein can be derived from any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). Retroviruses” of the present disclosure also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immnodeficiency virus (EIV), and other classes of retroviruses.

A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2); visna-maedi; a caprine arthritis-encephalitis virus; equine infectious anemia virus; feline immunodeficiency virus (FIV); bovine immune deficiency virus (BIV); and simian immunodeficiency virus (SIV). Preparation of the recombinant lentivirus can be achieved using the methods according to Dull et al. and Zufferey et al. (Dull et al., J. Virol., 1998; 72: 8463-8471 and Zufferey et al., J. Virol. 1998; 72:9873-9880).

Retroviral vectors (i.e., both lentiviral and non-lentiviral) for use in the present disclosure can be formed using standard cloning techniques by combining the desired DNA sequences in the order and orientation described herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

For expression of an anti-MAGE-A4 CAR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art, as described herein. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vectors provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The CARs of the present disclosure can be introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extrachromosomally. The nucleic acid may be maintained transiently or may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In certain embodiments, retroviral vectors are transduced by packaging the vectors into virions prior to contact with a cell. For example, a nucleic acid encoding an anti-MAGE-A4 CAR carried by a retroviral vector can be transduced into a cell through infection and pro virus integration.

As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably.

In particular, the CAR of the present disclosure is introduced and expressed in immune effector cells so as to redirect their specificity to a target antigen of interest, e.g., a malignant MAGE-A4-expressing cell, such as a malignant cell presenting MAGE-A4 with HLA-A2.

The present disclosure provides methods for making the immune effector cells which express the CAR as described herein. In one embodiment, the method comprises transfecting or transducing immune effector cells isolated from a subject, such as a subject having a MAGE-A4-expressing tumor cell, such that the immune effector cells express one or more CAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before or after being genetically modified (i.e., transduced or transfected to express a CAR as described herein).

Prior to in vitro manipulation or genetic modification of the immune effector cells described herein, the source of cells may be obtained from a subject. In particular, the immune effector cells for use with the CARs as described herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from the subject using any number of techniques known to the skilled person, such as FICOLL separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the present disclosure, the cells are washed with PBS. In an alternative embodiment, the washed solution lacks calcium, and may lack magnesium or may lack many, if not all, divalent cations. As would be appreciated by those of ordinary skill in the art, a washing step may be accomplished by methods known to those in the art, such as by using a semiautomated flowthrough centrifuge. After washing, the cells may be resuspended in a variety of biocompatible buffers or other saline solution with or without buffer. In certain embodiments, the undesirable components of the apheresis sample may be removed in the cell directly resuspended culture media.

In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present disclosure.

PBMCs may be used directly for genetic modification with the CARs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion. CD8+ cells can be obtained by using standard methods. In some embodiments, CD8+ cells are further sorted into naive, central memory, and effector cells by identifying cell surface antigens that are associated with each of those types of CD8+ cells. In embodiments, memory T cells are present in both CD62L+ and CD62L-subsets of CD8+ peripheral blood lymphocytes. PBMC are sorted into CD62L-CD8+ and CD62L+CD8+ fractions after staining with anti-CD8 and anti-CD62L antibodies. In some embodiments, the expression of phenotypic markers of central memory TCM include CD45RO, CD62L, CCR7, CD28, CD3, and CD127 and are negative for granzyme B. In some embodiments, central memory T cells are CD45RO+, CD62L+, CD8+ T cells. In some embodiments, effector T cells are negative for CD62L, CCR7, CD28, and CD127, and positive for granzyme B and perforin. In some embodiments, naive CD8+T lymphocytes are characterized by the expression of phenotypic markers of naive T cells including CD62L, CCR7, CD28, CD3, CD 127, and CD45RA.

In certain embodiments, CD4+ T cells are further sorted into subpopulations. For example, CD4+T helper cells can be sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+CD4+ T cell. In some embodiments, central memory CD4+ cells are CD62L positive and CD45RO positive. In some embodiments, effector CD4+ cells are CD62L and CD45RO negative.

The immune effector cells, such as T cells, can be genetically modified following isolation using known methods, or the immune effector cells can be activated and expanded (or differentiated in the case of progenitors) in vitro prior to being genetically modified. In another embodiment, the immune effector cells, such as T cells, are genetically modified with the chimeric antigen receptors described herein (e.g., transduced with a viral vector comprising a nucleic acid encoding a CAR) and then are activated and expanded in vitro. Methods for activating and expanding T cells are known in the art and are described, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). In other embodiments, the T cells may be activated and stimulated to proliferate with feeder cells and appropriate antibodies and cytokines using methods such as those described in U.S. Pat. Nos. 6,040,177; 5,827,642; and WO2012129514.

The present disclosure provides a population of modified immune effector cells for the treatment of a patient having a malignancy caused by a MAGE-A4-expressing tumor, e.g., multiple myeloma or melanoma, the modified immune effector cells comprising an anti-MAGE-A4 CAR as disclosed herein.

CAR-expressing immune effector cells prepared as described herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

A treatment-effective amount of cells in the composition is at least 2 cells (for example, at least 1 CD8+ central memory T cell and at least 1 CD4+ helper T cell subset) or is more typically greater than 10² cells, and up to 10⁶ up to and including 10⁸ or 10⁹ cells and can be more than 10¹⁰ cells. The number of cells will depend upon the ultimate use for which the composition is intended as will the type of cells included therein.

The cells may be autologous or heterologous to the patient undergoing therapy. If desired, the treatment may also include administration of mitogens (e.g., PHA) or lymphokines, cytokines, and/or chemokines (e.g., IFN-γ, IL-2, IL-12, TNF-α, IL-18, and TNF-β, GM-CSF, IL-4, IL-13, Flt3-L, RANTES, MIP1α, etc.) as described herein to enhance induction of the immune response.

The CAR expressing immune effector cell populations of the present disclosure may be administered either alone, or as a pharmaceutical composition in combination with diluents and/or with other components such as IL-2 or other cytokines or cell populations. Briefly, pharmaceutical compositions of the present disclosure may comprise a CAR-expressing immune effector cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The anti-tumor immune response induced in a subject by administering CAR expressing T cells described herein using the methods described herein, or other methods known in the art, may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

Thus, the present disclosure provides for methods of treating an individual diagnosed with or suspected of having, or at risk of developing a malignancy characterized at least in part by the expression of MAGE-A4 by cancer cells (e.g., MAGE-A4 expressing solid tumor cells), comprising administering to the individual a therapeutically effective amount of the CAR-expressing immune effector cells as described herein.

In one embodiment, the present disclosure provides a method of treating a subject diagnosed with a MAGE-A4-expressing cancer comprising removing immune effector cells from a subject diagnosed with a MAGE-A4-expressing cancer, genetically modifying said immune effector cells with a vector comprising a nucleic acid encoding a chimeric antigen receptor of the present disclosure, thereby producing a population of modified immune effector cells, and administering the population of modified immune effector cells to the same subject. In one embodiment, the immune effector cells comprise T cells.

The methods for administering the cell compositions described herein includes any method which is effective to result in reintroduction of ex vivo genetically modified immune effector cells that either directly express a CAR of the present disclosure in the subject or on reintroduction of the genetically modified progenitors of immune effector cells that on introduction into a subject differentiate into mature immune effector cells that express the CAR. One method comprises transducing peripheral blood T cells ex vivo with a nucleic acid construct in accordance with the present disclosure and returning the transduced cells into the subject.

### Binding Properties of the Chimeric Antigen Receptors and Corresponding Antibodies

As used herein, the term “binding” is discussed in the context of the binding of a chimeric antigen receptor or a corresponding antibody (or bispecific antibody) to, e.g., a predetermined antigen, such as a cell surface protein or fragment thereof (or to an antigen bound to a cell surface protein such as an HLA molecule). Binding typically refers to an interaction or association between a minimum of two entities or molecular structures, such as an antigen-binding domain:antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody or chimeric antigen receptor as the analyte (or antiligand). Cell-based binding strategies, such as fluorescent-activated cell sorting (FACS) binding assays, are also routinely used, and FACS data correlates well with other methods such as radioligand competition binding and SPR (Benedict, CA, J Immunol Methods. 1997, 201(2):223-31; Geuijen, C A, et al. J Immunol Methods. 2005, 302(1-2):68-77).

Accordingly, a chimeric antigen receptor or corresponding antibody (or bispecific antibody) of the present disclosure binds to the predetermined antigen or cell surface molecule (receptor) having an affinity corresponding to a K_{D} value that is at least ten-fold lower than its affinity for binding to a non-specific antigen (e.g., BSA, casein). As described herein, a chimeric antigen receptor or corresponding antibody of the present disclosure can bind to an HLA-presented MAGE-A4 antigen, e.g., an HLA-A2-presented MAGE-A4 antigen. According to the present disclosure, the affinity of a chimeric antigen receptor or a corresponding antibody with a K_{D} value that is equal to or less than ten-fold lower than a non-specific antigen may be considered non-detectable binding.

The term “K_{D}” (M) refers to the dissociation equilibrium constant of a particular antigen-binding domain:antigen interaction, or the dissociation equilibrium constant of a corresponding antibody to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g., a chimeric antigen receptor or a corresponding antibody) to its interactive partner molecule (e.g. antigen X) compared to the binding affinity of the molecule (e.g., chimeric antigen receptor or corresponding antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec−1 or 1/s) refers to the dissociation rate constant of a particular antigen-binding domain:antigen interaction, or the dissociation rate constant of a chimeric antigen receptor or a corresponding antibody. Said value is also referred to as the k_{off} value.

The term “kₐ” (M−1×sec−1 or 1/M) refers to the association rate constant of a particular antigen-binding domain:antigen interaction, or the association rate constant of a chimeric antigen receptor or a corresponding antibody.

The term “K_{A}” (M−1 or 1/M) refers to the association equilibrium constant of a particular antigen-binding domain:antigen interaction, or the association equilibrium constant of a chimeric antigen receptor or a corresponding antibody. The association equilibrium constant is obtained by dividing the kₐ by the k_{d}.

The term “EC50” or “EC₅₀” refers to the half maximal effective concentration, which includes the concentration of an antibody or chimeric antigen receptor that induces a response halfway between the baseline and maximum after a specified exposure time. The EC₅₀ essentially represents the concentration of a chimeric antigen receptor or antibody (e.g., bispecific antibody), where 50% of its maximal effect is observed. In certain embodiments, the EC₅₀ value equals the concentration of a chimeric antigen receptor or a corresponding antibody of the present disclosure that gives half-maximal binding to cells expressing an antigen (e.g., a tumor-associated antigen, such as MAGE-A4), as determined by e.g. a FACS binding assay and/or a T cell reporter/antigen presenting cell (APC) bioassay. Thus, reduced or weaker binding is observed with an increased EC₅₀, or half maximal effective concentration value.

In one embodiment, decreased binding can be defined as an increased EC₅₀ bispecific antibody, antigen-binding fragment, chimeric antigen receptor, or corresponding antibody concentration that enables binding to the half-maximal amount of target cells.

### Sequence Variants of the Antibodies and Chimeric Antigen Receptors

The antibodies, antigen-binding fragments, and chimeric antigen receptors of the present disclosure may comprise one or more amino acid substitutions, insertions and/or deletions in the framework region (FR) and/or complementarity determining region (CDR) of the heavy and light chain variable domains as compared to the corresponding germline sequences from which the individual antigen-binding domains of the corresponding antibodies were derived. Such mutations can be readily ascertained by comparing the amino acid sequences disclosed herein to germline sequences available from, for example, public antibody sequence databases. The antibodies, antigen-binding fragments and chimeric antigen receptors of the present disclosure may comprise antigen-binding domains which are derived from any of the exemplary CDR or variable region amino acid sequences disclosed herein, wherein one or more amino acids within one or more framework and/or CDR regions are mutated to the corresponding residue(s) of the germline sequence from which the corresponding antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies that comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence.

### Biological Characteristics of the Chimeric Antigen Receptors and Corresponding Antibodies

The present disclosure provides antibodies (including bispecific antibodies), antigen-binding fragments, and chimeric antigen receptors (CARs) with antigen-binding domains derived from antibodies that bind human MAGE-A4 or human MAGE-A4 and CD3. As described herein, bispecific antibodies can be used to generate CARs with similar properties as the bispecific antibody. Thus, the properties described herein and attributed to bispecific antibodies likewise apply to CARs. For example, the present disclosure includes anti-MAGE-A4 antibodies that bind human MAGE-A4 with an EC₅₀ value of less than 2 nM and a S/N ratio of greater than 1900 as assessed by a flow cytometry-based peptide pulsing assay as described infra with respect to Example 2. As another example, the present disclosure provides anti-MAGE-A4 antibodies that bind one or more MAGE-A4-related peptides with S/N ratios ranging from about 5 to greater than 300, as assessed by the flow cytometry-based peptide pulsing assay of Example 2, and as detailed in Example 3. As another example, the present disclosure provides anti-MAGE-A4×anti-CD3 bispecific antibodies that bind one or more of the MAGE-A4-related peptides with S/N ratios ranging from about 5 to greater than 240, as assessed by the flow-cytometry based peptide pulsing assay of Example 2, and as detailed in Example 3.

The present disclosure also provides anti-MAGE-A4×anti-CD3 bispecific antibodies that exhibit substantial activity in a T cell reporter/antigen presenting cell (APC) bioassay, as detailed infra in Example 4. Specifically, anti-MAGE-A4×anti-CD3 bispecific antibodies of the present disclosure can activate NF-κB-dependent T cell (e.g., Jurkat cells) gene transcription/translation as measured by luciferase expression in the presence of APCs expressing MAGE-A4 and HLA-A2 (e.g., IM9 and U266B1 cells) with EC₅₀ values of less than 3.2 nM (U266B1 cells) or less than 0.75 nM. Inclusion of an anti-CD28 antibody in the T cell reporter/APC bioassay can modulate the activity of the anti-MAGE-A4×anti-CD3 bispecific antibodies, as a function of endogenous levels of CD80 and CD86, as detailed infra in Example 4.

The present disclosure also provides anti-MAGE-A4×anti-CD3 bispecific antibodies that exhibit substantial activity in the T cell reporter/antigen presenting cell (APC) bioassay discussed above with one or more MAGE-A4-related peptides, as detailed infra in Example 5. Specifically, the anti-MAGE-A4×anti-CD3 bispecific antibodies exhibit subnanomolar EC₅₀ values (e.g., 2.10E-11 M for bispecific antibody with a high affinity CD3 arm, and 3.30E-10 M for bispecific antibody with a medium affinity CD3 arm) in terms of stimulated reporter activity when incubated in the presence of the T cells harboring the luciferase reporter and T2 cells pulsed with AOX₁₇₉₅₋₈₀₃. Additionally, the anti-MAGE-A4×anti-CD3 bispecific antibodies can exhibit nanomolar EC₅₀ values (e.g., 1.30E-9 M for the bispecific antibody with the high affinity CD3 arm, and 3.5E-9 M for the bispecific antibody with the medium affinity CD3 arm) in terms of stimulated reporter activity when incubated in the presence of the T cells harboring the luciferase reporter and T2 cells pulsed with SHTN₁₁₉₈₋₂₀₆.

The present disclosure also provides anti-MAGE-A4×anti-CD3 bispecific antibodies capable of directing a T cell (e.g., CD8+ T cells isolated from human PBMCs) response against tumor cells (e.g., MAGE-A₄₂₈₆₋₂₉₄ antigen presenting HLA-A2 positive IM9 cells), as assessed by an imaging based multiplexed primary T cell killing assay, as detailed infra in Example 6. Specifically, certain anti-MAGE-A4×anti-CD3 bispecific antibodies of the present disclosure are capable, at concentrations of less than 10 nM (e.g., 6.6 nM) of reducing tumor cell viability due to an antibody-dependent T cell-mediated response to less than 50% (e.g., 38%). This reduction in viability can be at least partially blocked by co-incubation of the anti-MAGE-A4×anti-CD3 bispecific antibody with an anti-CD28 antibody under circumstances where the tumor cells endogenously express CD80 and CD86, which may be due at least in part to the anti-CD28 antibody blocking CD28 interaction with CD80 and CD86.

The present disclosure also provides anti-MAGE-A4×anti-CD3 bispecific antibodies capable of stimulating cytokine release when incubated in the presence of tumor cells (e.g., MAGE-A4286-294 antigen presenting HLA-A2 positive IM9 cells) and T cells (e.g., CD8+ T cells isolated from human PBMCs), as detailed infra in Example 7. Specifically, the present disclosure provides anti-MAGE-A4×anti-CD3 bispecific antibodies capable of stimulating cytokine release (e.g., IL2, IFN-γ) by greater than 2-fold as compared to cytokine release under conditions where the tumor and T cells lack the anti-MAGE-A4×anti-CD3 bispecific antibody.

The present disclosure also provides chimeric antigen receptors with antigen-binding domains derived from corresponding antibodies that bind specifically to human cell lines which express endogenous MAGE-A4, as determined by a FACS binding assay.

The present disclosure also provides engineered cells expressing MAGE-A4-specific chimeric antigen receptors that (i) are activated by MAGE-A4-expressing cells (see Example 8), and/or (ii) exhibit inhibition of tumor growth in immunocompromised mice bearing human multiple myeloma or melanoma xenografts.

### Preparation of Antigen-Binding Domains

The antigen-binding domains of the antibodies (including bispecific antibodies) and chimeric antigen receptors of the present disclosure, which are specific for particular antigens (e.g., MAGE-A4), can be prepared by any antibody generating technology known in the art. In certain embodiments, one or more of the individual components (e.g., heavy and light chains) of the corresponding antibodies of the present disclosure are derived from chimeric, humanized or fully human antibodies. Methods for making such antibodies are well known in the art. For example, one or more of the heavy and/or light chains can be prepared using VELOCIMMUNE™ technology. Using VELOCIMMUNE™ technology (or any other human antibody generating technology), high affinity chimeric antibodies to a particular antigen (e.g., MAGE-A4) are initially isolated having a human variable region and a mouse constant region. The antibodies are characterized and selected for desirable characteristics, including affinity, neutralization, selectivity, epitope, etc. As discussed herein, these human variable regions (or CDRs) can then be incorporated into the antigen-binding domains of the chimeric antigen receptors. In other examples, two different antigens (e.g., anti-MAGE-A4 and anti-CD3) can be appropriately arranged relative to one another to produce a bispecific antigen-binding molecule of the present disclosure (e.g., and antibody, CAR, or antigen-binding fragment of either) by routine methods. In certain embodiments, one or more of the individual components (e.g., heavy and light chains) of the multispecific antigen-binding molecules of the present disclosure are derived from chimeric, humanized, or fully humanized antibodies.

### Polynucleotides and Vectors

The present disclosure also provides polynucleotides and vectors encoding the antibodies (or portions thereof) and chimeric antigen receptors discussed herein.

In various embodiments, the polynucleotide may comprise an expression cassette or expression vector (e.g., a plasmid for introduction into a bacterial host cell, or a viral vector such as a baculovirus vector for transfection of an insect host cell, or a plasmid or viral vector such as a lentivirus or adeno-associated virus for transfection of a mammalian host cell).

In various embodiments, the polynucleotides and/or vectors comprise a nucleic acid molecule comprising the nucleotide sequence of SEQ ID NO: 21, or comprise a nucleic acid molecule comprising a nucleotide sequence encoding the polypeptide sequence of SEQ ID NO: 22. In other embodiments, the polynucleotides and/or vectors comprise a nucleic acid molecule comprising the nucleotide sequence of SEQ ID NO: 104, or comprise a nucleic acid molecule comprising a nucleotide sequence encoding the polypeptide sequence of SEQ ID NO: 105. In various embodiments, the polynucleotides and/or vectors comprise a nucleic acid molecule comprising a nucleotide sequence of SEQ ID NO: 1, or comprise a nucleic acid molecule comprising a nucleotide sequence encoding the polypeptide sequence of SEQ ID NO: 2 or SEQ ID NO: 83. In various embodiments, the polynucleotides and/or vectors comprise a nucleic acid molecule comprising a nucleotide sequence of SEQ ID NO: 9, or comprise a nucleic acid molecule comprising a nucleotide sequence encoding the polypeptide sequence of SEQ ID NO: 10. In various embodiments, the polynucleotides and/or vectors comprise a nucleic acid molecule comprising a nucleotide sequence of SEQ ID NO: 17, or comprise a nucleic acid molecule comprising a nucleotide sequence encoding the polypeptide sequence of SEQ ID NO: 18. In various embodiments, the polynucleotides and/or vectors comprise a nucleic acid molecule comprising a nucleotide sequence of SEQ ID NO: 19, or comprise a nucleic acid molecule comprising a nucleotide sequence encoding the polypeptide sequence of SEQ ID NO: 20. In various embodiments, the polynucleotides and/or vectors comprise a nucleic acid molecule comprising a nucleotide sequence of SEQ ID NO: 54, or comprise a nucleic acid molecule comprising a nucleotide sequence encoding the polypeptide sequence of SEQ ID NO: 55. In various embodiments, the polynucleotides and/or vectors comprise a nucleic acid molecule comprising a nucleotide sequence of SEQ ID NO: 62, or comprise a nucleic acid molecule comprising a nucleotide sequence encoding the polypeptide sequence of SEQ ID NO: 63. In various embodiments, the polynucleotides and/or vectors comprise a nucleic acid molecule comprising a nucleotide sequence of SEQ ID NO: 68, or comprise a nucleic acid molecule comprising a nucleotide sequence encoding the polypeptide sequence of SEQ ID NO: 69. In various embodiments, the polynucleotides and/or vectors comprise a nucleic acid molecule comprising a nucleotide sequence of SEQ ID NO: 70, or comprise a nucleic acid molecule comprising a nucleotide sequence encoding the polypeptide sequence of SEQ ID NO: 71. In various embodiments, the polynucleotides and/or vectors comprise a nucleic acid molecule comprising a nucleotide sequence of SEQ ID NO: 72, or comprise a nucleic acid molecule comprising a nucleotide sequence encoding the polypeptide sequence of SEQ ID NO: 73. In various embodiments, the polynucleotides and/or vectors comprise a nucleic acid molecule comprising a nucleotide sequence of SEQ ID NO: 80, or comprise a nucleic acid molecule comprising a nucleotide sequence encoding the polypeptide sequence of SEQ ID NO: 81. With respect to the above-mentioned SEQ ID NOs, it is within the scope of this disclosure that the polynucleotides and/or vectors encompass any subsequence thereof, such as one or more HCDRs, LCDRs, etc. In various embodiments, the polynucleotides and/or vectors comprise a nucleotide sequence encoding the amino acid sequence of SEQ ID NO: 27, SEQ ID NO: 28, SEQ ID NO: 29, or SEQ ID NO: 30.

### Methods of Engineering Immune Cells Expressing Chimeric Antigen Receptors

The present disclosure provides methods of preparing immune cells for immunotherapy comprising introducing, ex vivo, into such immune cells the polynucleotides or vectors encoding one of the MAGE-A4-specific chimeric antigen receptors described herein. Such immune cells can be autologous or allogeneic.

The present disclosure provides immune cells comprising a polynucleotide or lentiviral vector encoding one of the MAGE-A4-specific chimeric antigen receptors discussed herein. In some embodiments, these immune cells are used for immunotherapy (e.g., treatment of cancer).

The present disclosure provides methods of genetically modifying immune cells to make them more suitable for allogeneic transplantation. According to a first aspect, the immune cell can be made allogeneic, for instance, by inactivating at least one gene expressing one or more component of T-cell receptor (TCR) as described in WO 2013/176915, which can be combined with the inactivation of a gene encoding or regulating HLA or B2m protein expression. Accordingly, the risk of graft versus host syndrome and graft rejection is significantly reduced. According to further aspect of the present disclosure, the immune cells can be further manipulated to make them more active or limit exhaustion, by inactivating genes encoding proteins that act as “immune checkpoints” that act as regulators of T-cells activation, such as PD1 or CTLA-4.

### Engineered Immune Cells

The present disclosure also provides immune cells (e.g., engineered immune cells) comprising a chimeric antigen receptor as described herein. In some cases, the immune cell is an immune effector cell. In some cases, the immune cell is a T cell. In some cases, the immune cell is a T lymphocyte selected from an inflammatory T lymphocyte, a cytotoxic T lymphocyte, a regulatory T lymphocyte, or a helper T lymphocyte. In some cases, the immune cell is a CD8+ cytotoxic T lymphocyte.

In some embodiments, the engineered immune cell is a human T cell comprising a chimeric antigen receptor comprising, from N-terminus to C-terminus: (a) an extracellular ligand-binding domain comprising an anti-MAGE-A4 single chain variable fragment (scFv) domain comprising a light chain variable region (LCVR) and a heavy chain variable region (HCVR); (b) a hinge; (c) a transmembrane domain; and (d) a cytoplasmic domain comprising a costimulatory domain and a signaling domain.

In some embodiments, the scFv domain of the engineered human T cell comprises a HCVR/LCVR amino acid sequence pair comprising the amino acid sequences of SEQ ID NOs: 2/10. In some cases, the hinge comprises the amino acid sequence of SEQ ID NO: 27. In some cases, the transmembrane domain comprises the amino acid sequence of SEQ ID NO: 28. In some cases, the costimulatory domain is a 4-1BB costimulatory domain. In some cases, the 4-1BB costimulatory domain comprises the amino acid sequence of SEQ ID NO: 29. In some cases, the signaling domain is a CD3zeta signaling domain. In some cases, the CD3zeta signaling domain comprises the amino acid sequence of SEQ ID NO: 30.

In various embodiments, the engineered human T cell comprises a chimeric antigen receptor comprising the amino acid sequence of SEQ ID NO: 22. In various embodiments, the engineered human T cell comprises a chimeric antigen receptor comprising the amino acid sequence of SEQ ID NO: 105.

### Bioequivalents

The present disclosure provides antibodies and chimeric antigen receptors, and engineered cells expressing the chimeric antigen receptors, which have amino acid sequences that vary from those of the exemplary molecules disclosed herein but that retain the ability to bind MAGE-A4 (and CD3 in the case of bispecific antibodies), activate immune cells expressing the chimeric antigen receptors in the presence of MAGE-A4-expressing cells, or suppress growth or proliferation of MAGE-A4-expressing tumor cells. Such variant molecules may comprise one or more additions, deletions, or substitutions of amino acids when compared to a parent sequence, but exhibit biological activity that is essentially equivalent to that of the described antigen-binding molecules.

In one embodiment, two engineered immune cells expressing a chimeric antigen receptor of the present disclosure, or two antigen-binding proteins of the present disclosure, are bioequivalent if there are no clinically meaningful differences in their safety, purity, and potency.

In one embodiment, two engineered immune cells, or two antigen-binding proteins, are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two engineered immune cells, or two antigen-binding proteins, are bioequivalent if they both act by a common mechanism or mechanisms of action for the condition or conditions of use, to the extent that such mechanisms are known.

In one embodiment, two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single doses or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

Bioequivalence may be demonstrated by in vivo and in vitro methods. Bioequivalence measures include, e.g., (a) an in vivo test in humans or other mammals, in which the concentration of the engineered cell is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the engineered cell (or its target) is measured as a function of time; and (d) in a well-controlled clinical trial that establishes safety, efficacy, or bioavailability or bioequivalence of an engineered cell.

Bioequivalent variants of the exemplary engineered cells set forth herein may be constructed by, for example, making various substitutions of residues or sequences or deleting terminal or internal residues or sequences not needed for biological activity.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

### Species Selectivity and Species Cross-Reactivity

According to certain embodiments of the present disclosure, antigen-binding domains are provided which bind to human MAGE-A4, but not to MAGE-A4 from other species. Also provided are anti-MAGE-A4 antigen-binding domains which also bind to human AOX1, but not to AOX1 from other species. Also provided are anti-MAGE-A4 antigen-binding domains which bind to human SHTN1, but not to SHTN1 from other species. The present disclosure also provides antigen-binding domains that bind to human MAGE-A4 and to MAGE-A4 from one or more non-human species. The present disclosure also provides anti-MAGE-A4 antigen-binding domains that bind to human AOX1 and to AOX1 from one or more non-human species. The present disclosure also provides anti-MAGE-A4 antigen-binding domains that bind to human SHTN1 and to SHTN1 from other species. In some embodiments, the antigen-binding domains of the present disclosure bind to MAGE-A4 286-294 as well as or AOX1 795-803 and/or SHTN1 198-206. In some embodiments, the MAGE-A4 and/or AOX1 and/or SHTN1 to which an antigen-binding domain binds (e.g., MAGE-A4 286-294 or AOX1 795-803 or SHTN1 198-206) is presented on the surface of a cell by an HLA, e.g., HLA-A2.

According to certain exemplary embodiments of the present disclosure, antigen-binding domains are provided which bind to human MAGE-A4 and/or MAGE-A4 related peptides and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee MAGE-A4 and/or MAGE-A4 related peptides. Moreover, the binding to MAGE-A4 and/or MAGE-A4 related peptides can be in the context of an MHC-presented MAGE-A4 (or MAGE-A4 related peptide), such as an HLA-presented MAGE-A4. An exemplary HLA-presented MAGE-A4 is HLA-A2-bound human MAGE-A4.

### Activation and Expansion of Engineered Immune Cells

Whether prior to or after genetic modification of the engineered cells (e.g., T cells), even if the genetically modified immune cells of the present disclosure are activated and proliferate independently of antigen binding mechanisms, the immune cells, particularly T-cells of the present disclosure can be further activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

Generally, the T cells of the present disclosure can be expanded by contact with an agent that stimulates a CD3 TCR complex and a costimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, IL-10, IL-2, 1L-15, TGFp, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% O₂). T cells that have been exposed to varied stimulation times may exhibit different characteristics.

In some embodiments, the cells can be expanded by co-culturing with tissue or cells. The cells can also be expanded in vivo, for example in the subject's blood after administrating said cell into the subject.

### Therapeutic Formulation and Administration

As used herein, the terms “effective amount” and “therapeutically effective amount” refer to the quantity of the active therapeutic agent sufficient to yield a desired therapeutic response without undue adverse side effects such as toxicity, irritation, or allergic response. The specific “effective amount” will, obviously, vary with such factors as the particular condition being treated, the physical condition of the patient, the type of animal being treated, the duration of the treatment, the nature of concurrent therapy (if any), and the specific formulations employed and the structure of the compounds or its derivatives. In this case, an amount would be deemed therapeutically effective if it resulted in one or more of, but not limited to, the following: (a) the inhibition of tumor growth (e.g., MAGE-A4-expressing cancer); and (b) the reversal or stabilization of a MAGE-A4-expressing cancer.

The dose of antigen-binding molecule administered to a patient may vary depending upon the age and the size of the patient, target disease, conditions, route of administration, and the like. The preferred dose is typically calculated according to body weight or body surface area. When an antibody or bispecific antigen-binding molecule of the present invention is used for therapeutic purposes in an adult patient, it may be advantageous to intravenously administer the bispecific antigen-binding molecule of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a bispecific antigen-binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

The administration of the cells or population of cells according to the present disclosure may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administered in one or more doses. In some embodiments, the effective amount of cells is administered as a single dose. In some embodiments, the effective amount of cells is administered as more than one dose over a period time. Timing of administration is within the judgment of managing physician and depends on the clinical condition of the patient. The cells or population of cells may be obtained from any source, such as a blood bank or a donor. While individual needs vary, determination of ranges of effective amounts of a given cell type for a particular disease or condition are within the skill of the art. An effective amount means an amount which provides a therapeutic or prophylactic benefit. The dosage administered will be dependent upon the age, health and weight of the recipient, kind of concurrent treatment, if any, frequency of treatment and the nature of the effect desired.

In one embodiment, the effective amount of cells or composition comprising those cells is administered parenterally. This administration can be an intravenous administration. In some cases, administration can be directly done by injection within a tumor.

In certain embodiments of the present disclosure, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efaliztimab treatment for psoriasis patients or other treatments for PML patients. In further embodiments, the T cells of the present disclosure may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation.

In a further embodiment, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present disclosure are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present disclosure. In an additional embodiment, expanded cells are administered before or following surgery. In certain embodiments, any means (e.g., surgery, chemotherapy, or radiation therapy) may be used to reduce the tumor burden prior to administration of the expanded immune cells of the present disclosure. In one embodiment, reducing the tumor burden prior to administration of the engineered cells of the present disclosure can reduce the potential for, or prevent, cytokine release syndrome or a cytokine storm, a side effect that may be associated with CAR T cell therapy.

### Therapeutic Applications

The present disclosure provides compositions comprising an engineered cell (e.g., a T cell) expressing a chimeric antigen receptor of the present disclosure and a pharmaceutically acceptable vehicle. The present disclosure also provides compositions comprising an antibody, or an antigen-binding fragment thereof, and a pharmaceutically acceptable vehicle. In some cases, the engineered cells, antibodies, or antigen-binding fragments form a medicament, particularly for immunotherapy. In some cases, the engineered cells, antibodies, or antigen-binding fragments are used for the treatment of cancer (e.g., multiple myeloma or melanoma). In some cases, the engineered cells, antibodies, or antigen-binding fragments are used in the manufacture of a medicament for immunotherapy and/or the treatment of a cancer (e.g., a MAGE-A4-expressing cancer).

The present disclosure provides methods comprising administering to a subject in need thereof a therapeutic composition comprising an antibody (e.g., a bispecific antibody), or antigen-binding fragment thereof, and/or an engineered cell (e.g., a T cell) expressing a chimeric antigen receptor as discussed herein. The therapeutic composition can comprise a cell expressing any chimeric antigen receptor as disclosed herein and a pharmaceutically acceptable carrier, diluent or vehicle. In additional or alternative examples, the therapeutic composition comprises an antibody and/or antigen-binding fragment as discussed herein. As used herein, the expression “a subject in need thereof” means a human or non-human animal that exhibits one or more symptoms or indicia of cancer (e.g., a subject with a MAGE-A4-expressing tumor or suffering from any of the cancers mentioned herein), or who otherwise would benefit from an inhibition or reduction in MAGE-A4 activity or a depletion of MAGE-A4+ cells.

The engineered cells and/or antibodies and antigen-binding fragments of the present disclosure can be useful, inter alia, for treating any disease or disorder in which stimulation, activation and/or targeting of an immune response would be beneficial. In particular, the engineered cells and/or antibodies and antigen-binding fragments of the present disclosure may be used for the treatment, prevention and/or amelioration of any disease or disorder associated with or mediated by MAGE-A4 expression or activity or the proliferation of MAGE-A4+ cells. Cells expressing MAGE-A4 which can be inhibited or killed using the engineered cells and/or antibodies and antigen-binding fragments of the present disclosure include, for example, multiple myeloma cells, melanoma cells, or other solid tumor cells.

The engineered cells and/or antibodies and antigen-binding fragments of the present disclosure may be used to treat a disease or disorder associated with MAGE-A4 expression including, e.g., a cancer including but not limited to multiple myeloma, synovial sarcoma, esophageal cancer, head and neck cancer, lung cancer, bladder cancer, ovarian cancer, uterine cancer, stomach cancer, cervical cancer, breast cancer, and melanoma. The engineered cells and/or antibodies and antigen-binding fragments of the present disclosure may generally be used to treat a tumor that expresses MAGE-A4. According to other related embodiments of the present disclosure, methods are provided comprising administering an engineered cell and/or antibodies and antigen-binding fragments as disclosed herein to a patient who is afflicted with a tumor expressing MAGE-A4, including tumors from the cancers listed above. Analytic/diagnostic methods known in the art, such as tumor scanning, etc., may be used to ascertain whether a patient harbors such a tumor, disease, or condition.

The present disclosure also provides methods for treating residual cancer in a subject. As used herein, the term “residual cancer” means the existence or persistence of one or more cancerous cells in a subject following treatment with an anti-cancer therapy.

According to certain aspects, the present disclosure provides methods for treating a disease or disorder associated with MAGE-A4 expression (e.g., cancer) comprising administering a population of engineered cells and/or antibodies and antigen-binding fragments described elsewhere herein to a subject after the subject has been determined to have the disease or disorder. For example, the present disclosure provides methods for treating a disease or disorder comprising administering engineered immune cells to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received other immunotherapy or chemotherapy.

The treatments discussed herein can be ameliorating, curative or prophylactic. Treatments may be either part of an autologous immunotherapy or part of an allogeneic immunotherapy. By autologous, it is meant that the cells, cell line or population of cells used for treating patients are originating from the patient or from a Human Leucocyte Antigen (HLA) compatible donor. By allogeneic is meant that the cells, cell line or population of cells used for treating patients are not originating from the patient but from a donor.

Cells that can be used with the disclosed methods are described herein. The treatments can be used to treat patients diagnosed with a pre-malignant or malignant cancer condition characterized by MAGE-A4-expressing cells, especially by an overabundance of MAGE-A4-expressing cells. Such conditions can be found in cancers.

Types of cancers to be treated with the engineered cells, antibodies, and antigen-binding fragments of the present disclosure include, but are not limited to, multiple myeloma, synovial sarcoma, esophageal cancer, head and neck cancer, lung cancer, bladder cancer, ovarian cancer, uterine cancer, stomach cancer, cervical cancer, breast cancer, and melanoma.

Compositions and methods of the present disclosure may be used to treat a subject who has been characterized as having cells or tissues expressing MAGE-A4, or is suspected of having cells or tissues expressing MAGE-A4. For example, subjects benefiting from treatment according to the present disclosure include subjects with multiple myeloma, synovial sarcoma, esophageal cancer, head and neck cancer, lung cancer, bladder cancer, ovarian cancer, uterine cancer, stomach cancer, cervical cancer, breast cancer, or melanoma.

### Combination Therapies

The present disclosure provides methods which comprise administering antibodies/antigen-binding fragments (e.g., bispecific antibodies) and/or engineered cells or a population of cells comprising any of the chimeric antigen receptors described herein in combination with one or more additional therapeutic agents. Exemplary additional therapeutic agents that may be combined with or administered in combination with the antibodies/antigen-binding fragments and/or cells or population of cells of the present disclosure include, e.g., an anti-tumor agent (e.g. chemotherapeutic agents including melphalan, vincristine (Oncovin), cyclophosphamide (Cytoxan), etoposide (VP-16), doxorubicin (Adriamycin), liposomal doxorubicin (Doxil), obendamustine (Treanda), or any others known to be effective in treating a plasma cell tumor in a subject). In some embodiments, the second therapeutic agent comprises steroids. In some embodiments, the second therapeutic agent comprises targeted therapies including thalidomide, lenalidomide, and bortezomib, which are therapies approved to treat newly diagnosed patients. For example. Ienalidomide, pomalidomide, bortezomib, carfilzomib, panobinostat, ixazomib, elotuzumab, and daratumumab are examples of a second therapeutic agent effective for treating recurrent myeloma. In certain embodiments the second therapeutic agent is a regimen comprising radiotherapy or a stem cell transplant. In certain embodiments, the second therapeutic agent may be an immunomodulatory agent. In certain embodiments, the second therapeutic agent may be a proteasome inhibitor, including bortezomib (Velcade®), carfilzomib (Kyprolis®), ixazomib (Ninlaro®). In certain embodiments the second therapeutic agent may be a histone deacetylase inhibitor such as panobinostat (Farydak®). In certain embodiments, the second therapeutic agent may be a monoclonal antibody, an antibody drug conjugate, a bispecific antibody which may or may not be conjugated to an anti-tumor agent, a checkpoint inhibitor, or combinations thereof. Other agents that may be beneficially administered in combination with the antigen-binding molecules of the present disclosure include cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors. The pharmaceutical compositions of the present disclosure (e.g., pharmaceutical compositions comprising engineered cells or populations of cells as disclosed herein) may also be administered as part of a therapeutic regimen comprising one or more therapeutic combinations selected from a monoclonal antibody other than those described herein, which may interact with a different antigen on the plasma cell surface, a bispecific antibody, which has one arm that binds to an antigen on the tumor cell surface and the other arm binds to an antigen on a T cell, an antibody drug conjugate, a bispecific antibody conjugated with an anti-tumor agent, a checkpoint inhibitor, for example, one that targets, PD-1 or CTLA-4, or combinations thereof. In certain embodiments, the checkpoint inhibitors may be selected from PD-1 inhibitors, such as pembrolizumab (Keytruda®), nivolumab (Opdivo®), or cemiplimab (Libtayo®). In certain embodiments, the checkpoint inhibitors may be selected from PD-L1 inhibitors, such as atezolizumab (Tecentriq®), avelumab (Bavencio®), or Durvalumab (Imfinzi®). In certain embodiments, the checkpoint inhibitors may be selected from CTLA-4 inhibitors, such as ipilimumab (Yervoy®).

The present disclosure also includes therapeutic combinations comprising any of the antibodies/antigen-binding fragments and/or engineered cells or populations of cells mentioned herein and an inhibitor of one or more of VEGF, Ang2, DLL4, EGFR, ErbB2, ErbB3, ErbB4, EGFRvIII, cMet, IGF1R, B-raf, PDGFR-α, PDGFR-β, FOLHI (PSMA), PRLR, STEAP1, STEAP2, TMPRSS2, MSLN, CA9, uroplakin, or any of the aforementioned cytokines, wherein the inhibitor is an aptamer, an antisense molecule, a ribozyme, an siRNA, a peptibody, a nanobody or an antibody fragment (e.g., Fab fragment; F(ab′)₂ fragment; Fd fragment; Fv fragment; scFv; dAb fragment; or other engineered molecules, such as diabodies, triabodies, tetrabodies, minibodies and minimal recognition units). In some embodiments, the antibodies/antigen-binding fragments and/or engineered cells or population of cells of the present disclosure may also be administered as part of a treatment regimen that also includes radiation treatment and/or conventional chemotherapy.

The additional therapeutically active component(s) may be administered just prior to, concurrent with, or shortly after the administration of the engineered cells of the present disclosure; (for purposes of the present disclosure, such administration regimens are considered the administration of the engineered cells “in combination with” an additional therapeutically active component).

The present disclosure provides pharmaceutical compositions in which an engineered cell or population of cells of the present disclosure is co-formulated with one or more of the additional therapeutically active component(s) as described elsewhere herein.

### Administration Regimens

According to certain embodiments of the present disclosure, multiple doses of the antibodies/antigen-binding fragments and/or engineered cells may be administered to a subject over a defined time course. The methods according to this aspect comprise sequentially administering to a subject multiple doses of the antigen-binding molecules and/or cells. As used herein, “sequentially administering” means that each dose is administered to the subject at a different point in time, e.g., on different days separated by a predetermined interval (e.g., hours, days, weeks or months). The present disclosure provides methods which comprise sequentially administering to the patient a single initial dose, followed by one or more secondary doses, and optionally followed by one or more tertiary doses.

The terms “initial dose,” “secondary doses,” and “tertiary doses,” refer to the temporal sequence of administration of the antigen-binding molecules and/or engineered cells of the present disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of antigen-binding molecules and/or engineered cells, but generally may differ from one another in terms of frequency of administration. In certain embodiments, however, the amount of antigen-binding molecules and/or engineered cells contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose which is administered to a patient prior to the administration of the very next dose in the sequence with no intervening doses.

The methods according to this aspect of the present disclosure may comprise administering to a patient any number of secondary and/or tertiary doses. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### EXAMPLES

The following examples are put forth so as to provide those of ordinary skill in the art with a complete disclosure and description of how to make and use the methods and compositions of the present disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Anti-MAGE-A4 Antibodies

Anti-MAGE-A4 antibodies were obtained by immunizing a genetically modified mouse (e.g., an engineered mouse comprising DNA encoding human immunoglobulin heavy and kappa light chain variable regions) with a human MAGE-A4 antigen (e.g., hMAGE-A4 286-294 for mAb31339N2) and HLA-A2 (HLA-A*02:01). More particularly, the genome of the genetically modified mouse had a nucleotide sequence encoding human HLA-A2 (as well as the sequences encoding human immunoglobulin heavy and kappa light chain variable regions), such that the genetically modified mouse expressed the human HLA-A2, wherein the mouse was tolerized to the human HLA-A2 such that they generated a specific B-cell response when immunized with the MAGE-A4 antigen and HLA-A2.

Following immunization, splenocytes were harvested from each mouse and either (1) fused with mouse myeloma cells to preserve their viability and form hybridoma cells and screened for MAGE-A4 specificity, or (2) B-cell sorted (as described in US Patent Pub. No. 2007/0280945A1) using a human MAGE-A4 fragment as the sorting reagent that binds and identifies reactive antibodies (antigen-positive B cells).

Chimeric antibodies to MAGE-A4 were initially isolated having a human variable region and a mouse constant region. The antibodies were characterized and selected for desirable characteristics, including affinity, selectivity, etc. In examples, mouse constant regions were replaced with a desired human constant region, for example wild-type or modified IgG1 or IgG4 constant region, to generate a fully human anti-MAGE-A4 antibody (e.g., mAbM31339N2 (containing a mouse constant region) was used to generate mAbH31339N2 (containing a human constant region)). While the constant region selected may vary according to specific use, high affinity antigen-binding and target specificity characteristics reside in the variable region.

mAbM31339N2 was also reformatted into bispecific antibodies using either a high affinity (7195P) CD3 arm to generate bsAb6054 (HCVR1, LCVR1, and LCVR2 from mAb31339 and HCVR2 from 7195P) or a medium affinity (7221G) CD3 arm to generate bsAb6043 (HCVR1, LCVR1, and LCVR2 from mAb31339 and HCVR2 from 7221G).

Heavy and Light Chain Variable Region Amino Acid and Nucleic Acid Sequences of anti-MAGE-A4 antibodies: Table 1 sets forth the amino acid sequence identifiers of the heavy and light chain variable regions and CDRs of selected anti-MAGE-A4 antibodies of the present disclosure. The mAb31339N and mAb31339N2 sequences of Table 1 are identical except for one amino acid difference in the HCVR framework region 3; however the CDRs are the same for both mAb31339N and mAb31339N2. The corresponding nucleic acid sequence identifiers are set forth in Table 2. A summary of all sequences contained herein is provided in Table 15.

TABLE 1
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb31339N | 83 | 4 | 6 | 8 | 10 | 12 | 14 | 16 |
| mAbM31339N2 | 2 | 4 | 6 | 8 | 10 | 12 | 14 | 16 |
| mAbH31339N2 | 2 | 4 | 6 | 8 | 10 | 12 | 14 | 16 |
| mAbM34852N | 107 | 109 | 111 | 113 | 115 | 117 | 14 | 119 |

TABLE 2
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAbM31339N | 82 | 3 | 5 | 7 | 9 | 11 | 13 | 15 |
| mAbM31339N2 | 1 | 3 | 5 | 7 | 9 | 11 | 13 | 15 |
| mAbH31339N2 | 1 | 3 | 5 | 7 | 9 | 11 | 13 | 15 |
| mAbM34852N | 106 | 108 | 110 | 112 | 114 | 116 | 13 | 118 |

Generation of Bispecific Antibodies that bind CD3 and MAGE-A4: Bispecific antibodies comprising an anti-CD3-specific binding domain and an anti-MAGE-A4 specific binding domain were constructed with sequences listed in Table 3 and Table 4 using methodologies wherein a heavy chain from an anti-CD3 antibody was combined with a heavy chain and a cognate light chain from an anti-MAGE-A4 antibody.

Accordingly, the bispecific antibodies created in accordance with the present Example comprise two separate antigen-binding domains (i.e., binding arms). The first antigen-binding domain comprises a heavy chain variable region derived from an anti-MAGE-A4 antibody (“MAGE-A4-VH”), paired with a cognate light chain variable region derived from the anti-MAGE-A4 antibody (“MAGE-A4-VL”), and the second antigen-binding domain comprises a heavy chain variable region derived from an anti-CD3 antibody (“CD3-VH”) paired with the MAGE-A4-VL. In principle, it is also possible to use the cognate light chain variable region from the anti-CD3 antibody (“CD3-VL”) as the light chain variable region common to both arms of the antibody. The same MAGE-A4-VH was used in all bispecific antibodies created in this Example The bsAb6054 antibody comprises a MAGE-A4 binding arm comprising an HCVR/LCVR of SEQ ID NOs: 2/10 and a CD3 binding arm comprising an HCVR/LCVR of SEQ ID NOs: 73/10. The bsAb6043 antibody comprises a MAGE-A4 binding arm comprising an HCVR/LCVR of SEQ ID NOs: 2/10 and a CD3 binding arm comprising an HCVR/LCVR of SEQ ID NOs: 55/10.

The amino acid sequence identifiers of the heavy and light chain variable regions and CDRs used to construct the anti-CD3 antigen-binding arm and anti-MAGE-A4 binding arm are set forth in Table 3. The corresponding nucleic acid sequence identifiers are set forth in Table 4. The anti-MAGE/anti-CD3 bispecific antibodies were generated from either a medium-affinity CD3 antibody (anti-CD3-A; referred to herein as H4sH7221G or 7221G), or a high affinity CD3 antibody (anti-CD3-B; referred to herein as HpH4sH7195P or 7195P). The first antigen-binding domain and the second antigen-binding domain are herein referred to as “arms” of a bispecific antibody. In the Examples, one arm comprises a HCVR comprising the amino acid sequence of SEQ ID NO: 2, and an LCVR comprising the amino acid sequence of SEQ ID NO: 10, while the other arm comprises a HCVR comprising the amino acid sequence of SEQ ID NO: 55, or SEQ ID NO: 73, and an LCVR comprising the amino acid sequence of SEQ ID NO: 10.

TABLE 3
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAbM31339N2 | 2 | 4 | 6 | 8 | 10 | 12 | 14 | 16 |
| Anti-CD3-A | 55 | 57 | 59 | 61 | 63 | 65 | 14 | 67 |
| Anti-CD3-B | 73 | 75 | 77 | 79 | 63 | 65 | 14 | 67 |

TABLE 4
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAbM31339N2 | 1 | 3 | 5 | 7 | 9 | 11 | 13 | 15 |
| Anti-CD3-A | 54 | 56 | 58 | 60 | 62 | 64 | 13 | 66 |
| Anti-CD3-B | 72 | 74 | 76 | 78 | 62 | 64 | 13 | 66 |

### Example 2: Binding of Anti-HLA-A2:MAGE-A4 (286-294) Antibody to T2 Cells Pulsed with MAGE-A4 (286-294) Via Flow Cytometry

Cell surface binding of the anti-HLA-A2: MAGE-A4 286-294 antibody (mAbM31339N) to HLA-A*02:01 positive T2 (174 CEM.T2) cells was assessed in a flow cytometry-based peptide pulsing assay. To pulse, 1×10⁶ T2 cells were incubated with 10 μg/ml of human (h) B2M (EMD Millipore Cat #475828) and 100 μg/ml of a MAGE-A4 286-294 peptide in 1 ml of AIM V medium (Gibco. Cat #31035-025) for 16 h at 37° C. Cells were washed in staining buffer ((PBS without Calcium and Magnesium (Corning, Ref #21-031-CV)+2% FBS (Seradigm, Lot #238B15)), harvested using cell dissociation buffer (Millipore, Cat #S-004-C), and resuspended in staining buffer. Pulsed Cells (200,000) were plated in 96 well V-Bottom plates (Axygen, Cat #P-96-450V-C-S) and stained with three-fold serial dilutions (1.7 pM-100 nM) of mAbM31339N or a non-binding control antibody (mAb1097) for 30 mins at 4° C. Cells were then washed once in staining buffer, and incubated with an Alexa Fluor 647 conjugated Fab′2 anti-mouse Fc-specific secondary antibody (Jackson ImmunoResearch, Cat #115-606-071) at 5 ug/ml for 30 mins at 4° C. Finally, cells were stained with a green fluorescent viability dye (Molecular Probes cat #L-34970 reconstituted in 50 ul DMSO) at a concentration of 1:1000. Cells were then washed and fixed using a 50% solution of BD Cytofix (BD, Cat #554655) diluted in PBS. Samples were run on an Intellicyt iQue flow cytometer (Intellicyt) and results were analyzed using Forecyte analysis software (Intellicyte) to calculate the mean fluorescent intensity (MFI) after gating for live cells. MFI values were plotted in Graphpad Prism using a four-parameter logistic equation over a 12-point response curve to calculate EC₅₀ values. The secondary antibody alone (i.e. no primary antibody) for each dose-response curve is also included in the analysis as a continuation of the three-fold serial dilution and is represented as the lowest dose. The signal to noise (S/N) was determined by taking the ratio of the highest MFI on the dose response curve to the MFI in the secondary alone wells. The EC₅₀ values (M) and max S/N are shown in Table 5. mAbM31339N bound with an EC50 of 1.7 nM and a max S/N of 1933, whereas binding of the control antibody (mAb1097) was not detected, indicating that mAb31339N has strong binding affinity to MAGE-A4 (286-294). With reference to Table 5, ND refers to EC50 values that could not be accurately determined because the binding did not reach saturation within the tested antibody concentration range.

TABLE 5
Binding of Anti-HLA-A2:MAGE-A4 (286-294) Antibody
to T2 cells presenting MAGE-A4 (286-294)

| mAb | EC50 | Max S/N |
|---|---|---|
| mAbM31339N | 1.7E−09 | 1933 |
| mAb1097 (mIgG2a | ND | 1 |
| Isotype Control) | | |

### Example 3: Binding of Anti-HLA-A2:MAGE-A4 Antibody and MAGE-A4×CD3 Bispecific Antibodies to T2 Cells Pulsed with MAGE-A4-Related Peptides

An in-silico computational strategy (Dhanik A et. al. BMC Bioinformatics 2016) identified several MAGE-A4-related peptides predicted to form complexes with HLA-A*02:01. The identified peptides are summarized in Table 6.

TABLE 6
Sequences of MAGE-A4 286-294 Peptide and
Predicted Off-Target Peptides

| Peptide (Gene Name and | | |
|---|---|---|
| Peptide Location) | Peptide Sequence | SEQ ID NO |
| MAGE-A4 286-294 | KVLEHVVRV | 33 |
| FN1 2182-2190 | KVREEVVTV | 40 |
| AOX1 795-803 | KVMCHVRRV | 41 |
| PSME2 145-153 | KVLERVNAV | 42 |
| PCDHGC3 337-345 | KVLVEVVDV | 43 |
| SHTN1 198-206 | KVLEKCNRV | 44 |
| SMIM11A 4-12 | KVLEHVPLL | 45 |
| RABGAP1L 408-416 | FLLETVVRV | 46 |
| CNOT1 1965-1973 | KVLGIVVGV | 47 |
| CARD8 170-178 | MVLEHPARV | 48 |
| MRPL37 334-342 | KVLEQPVVV | 49 |
| PLEKHH2 790-798 | KVLQNVLRV | 50 |
| NBAS 2063-2071 | KVLEGVVAA | 51 |
| TEP1 2156-2164 | AVEEHVVSV | 52 |
| HEATR5A 1925-1933 | KVLETLVTV | 53 |

Binding of the parent antibody (mAbH31339N2), MAGE-A4×CD3 (7221G) bispecific antibody (bsAb6043), and a non-binding isotype control antibody (mAb4241) to these related peptides shown at Table 6 was assessed in the T2 pulsing assay described above. As noted above in Example 1, the bsAb6043 antibody comprises a MAGE-A4 binding arm comprising an HCVR/LCVR of SEQ ID NOs: 2/10 and a CD3 binding arm comprising an HCVR/LCVR of SEQ ID NOs: 55/10. As summarized in Table 7, both antibodies bound MAGE-A4 peptide with S/N values of 310 for mAbH31339N2 and 244 for bsAb6043, substantially above baseline (no peptide). The tested antibodies demonstrated lower binding to AOX1 with S/N values of 131 for mAbH31339N2 and 110 and for bsAb6043, and to SHTN1 with S/N values of 5 for both antibodies. No detectable binding to the remaining peptides was observed and the control antibody binding was ≤3 for all tested peptides.

TABLE 7
Binding of Anti-HLA-A2:MAGE-A4 (286-294) Antibody
to T2 cells presenting MAGE-A4-related peptides

| | Antibody | | |
|---|---|---|---|
| Peptide (Gene name | mAbH31339N2 | bsAb6043 | mAb4241 |
| and Peptide Location) | (S/N ratio) | (S/N ratio) | (S/N ratio) |
| No peptide | 1 | 1 | 3 |
| MAGE-A4 (286-294) | 310 | 244 | 3 |
| FN1 (2182-2190) | 1 | 1 | 3 |
| AOX1 (795-803) | 131 | 110 | 3 |
| PSME2 (145-153) | 1 | 1 | 3 |
| PCDHGC3 (337-345) | 1 | 1 | 3 |
| SHTN1 (198-206) | 5 | 5 | 3 |
| SMIM11A (4-12) | 1 | 1 | 3 |
| RABGAP1L (408-416) | 1 | 1 | 3 |
| CNOT1 (1965-1973) | 1 | 1 | 3 |
| CARD8 (170-178 | 1 | 1 | 2 |
| MRPL37 (334-342) | 1 | 1 | 3 |
| PLEKHH2 (790-798) | 1 | 1 | 2 |
| NBAS (2063-2071) | 1 | 1 | 3 |
| TEP1 (2156-2164) | 1 | 1 | 3 |
| HEATR5A (1925-1933) | 1 | 1 | 3 |

### Example 4: Assessment of Activity of the MAGE-A4×CD3 Bispecific Antibodies in a T Cell Reporter/Antigen Presenting Cell (APC) Bioassay

The activities of MAGE-A4×CD3 bispecific antibodies were assessed in a T cell reporter/antigen presenting cell (APC) bioassay. To generate the assay cell line, Jurkat cells were transduced with a NF-κB-dependent firefly luciferase lentiviral reporter vector (Qiagen) and single cell sorted for high luciferase activity to generate the Jurkat/NF-κBLuc assay cell line. IM9 and U266B1 cells, which are myeloma cell lines that endogenously express MAGE-A4 and HLA-A*02:01, were utilized as antigen presenting cells (APCs). In addition, Raji cells, which are MAGE-A4 and HLA-A2 negative, were used as a control. Briefly, 25,000 Jurkat/NF-κBLuc cells were added to Thermo-Nunc 96 well white plates (Thermo Scientific, Cat #136101) in 25 ul of assay media (RPMI media with 10% FBS and 1% P/S/G) followed by the addition of 25,000 APCs in 25 μl assay media. A 3-fold serial dilution of antibody from 27.4 pM-20 nM was added to the plate in 50 ul of assay media. The cell mixture was incubated in a 37° C., 5% CO₂, humidified incubator for 5 hours. NF-κB-Luciferase activity was measured using Promega One-Glo (Cat #E6130) and a Perkin Elmer Envision plate reader according to manufacturer instructions. Relative luciferase units (RLU) were generated and EC50 values were determined using a four-parameter logistic equation over an 8-point dose response curve (GraphPad Prism). The zero primary antibody condition (secondary antibody alone) for each dose-response curve is also included in the analysis as a continuation of the three-fold serial dilution and is represented as the lowest dose. Max Activity was determined by taking the ratio of the highest RLU on the curve to the lowest and is represented as signal:noise (S/N). The EC50 values and S/N are summarized in Table 8. With regard to Table 8, ND corresponds to EC50 values that could not be accurately determined because the binding did not reach saturation within the tested antibody concentration range.

TABLE 8
Activity of MAGE-A4 × CD3 Bispecific Antibodies in Jurkat/NF-κB-Luc/APC reporter Bioassay

| | | | Jurkat/NF-κB Luc | | Jurkat/NF-κB Luc | | Jurkat/NF-κB Luc | |
|---|---|---|---|---|---|---|---|---|
| | | | cl.1C22 + IM-9 | | cl.1C22 + U266 | | cl.1C22 + RAJI | |
| | | | EC50 | Fold | EC50 | Fold | EC50 | Fold |
| Antibody | VH1 | VH2 | (nM) | Activation | (nM) | Activation | (nM) | Activation |
| bsAb6043 | mAb31339N2 | mAb7221G | N.D. | 1.8 | N.D | 0.9 | N.D. | 0.8 |
| bsAb6054 | mAb31339N2 | mAb7195P | 7.40E−10 | 41.9 | 3.10E−09 | 6.3 | N.D | 0.9 |
| bsAb4241 | mAb8767P2 | mAb7221G | N.D. | 1.1 | N.D. | 1.1 | N.D | 0.9 |
| bsAb3905 | mAb17363N | mAb7195P | N.D. | 2.2 | N.D. | 1.2 | N.D | 0.9 |

As noted in Example 1, the bsAb6054 antibody comprises a MAGE-A4 binding arm comprising an HCVR/LCVR of SEQ ID NOs: 2/10 and a CD3 binding arm comprising an HCVR/LCVR of SEQ ID NOs: 73/10, and the bsAb6043 antibody comprises a MAGE-A4 binding arm comprising an HCVR/LCVR of SEQ ID NOs: 2/10 and a CD3 binding arm comprising an HCVR/LCVR of SEQ ID NOs: 55/10. The bsAb4241 and bsAb3905 antibodies are non-binding (to MAGE-A4) controls with anti-CD3 binding arms comprising the same anti-CD3 HCVR as bsAb6043 and bsAb6054, respectively, paired with a cognate light chain variable region from the non-MAGE-A4 binding arm of each respective antibody.

As shown at Table 8, the MAGE-A4×CD3 medium affinity bispecific antibody (bsAb6043) had no activity in the Jurkat/NF-κBLuc bioassay in the presence of IM9, U266B1, or RAJI cells. In contrast, the MAGE-A4×CD3 high affinity bispecific antibody (bsAb6054) had EC50 values of 7.4E-10M and 3.1E-09M and S/N values of 41.9 and 6.3 on IM9 and U266B1 cells, respectively. Non-binding control bispecific antibodies with either a medium affinity CD3 arm (mAb4241) or a high affinity CD3 arm (mAb3905) were minimally active with S/N values ≤2.2. Additionally, there was no activity of any antibody on RAJI cells.

A similar bioassay was performed, this time adding a constant amount of an anti-CD28 antibody. Here, the addition of CD28 partially blocked activity of bsAb6054 on IM9 cells (EC50 2.1E09M, S/N 28.7) while slightly increasing activity on U266B1 cells (EC50 6.1E-09M, S/N 8.6). This is likely attributed to the level of CD80 and CD86 co-stimulation molecules endogenously expressed on these cell lines. IM9 endogenously has high levels of CD80 and CD86. Thus, the addition of a CD28 antibody in the bioassay blocks the natural interaction of CD28 on the Jurkat cells with its ligands CD80 and CD86, therefore lowering activity. U266B1 has low levels of CD80 and CD86. Here, the addition of CD28 stimulates CD28 signaling on the Jurkat cells, increasing activity (Table 9).

TABLE 9
Activity of MAGE-A4 × CD3 Bispecific Antibodies in combination with
2 nM mAb5705 (anti-CD28 antibody) in Jurkat/NF-κB-Luc/APC reporter Bioassay

| | | | Jurkat/NF-κB Luc | | Jurkat/NF-κB Luc | |
|---|---|---|---|---|---|---|
| | | | cl.1C11 + IM-9 | | cl.1C11 + U266 | |
| | | | | Fold | | Fold |
| Antibody | VH1 | VH2 | EC50 (nM) | Activation | EC50 (nM) | Activation |
| bsAb6043 + 2 | mAb31339N2 | mAb7221G | N.D. | 1.1 | N.D. | 0.9 |
| nM mAb5705 | | | | | | |
| (CD28) | | | | | | |
| bsAb6054 + 2 | mAb31339N2 | mAb7195P | 2.10E−09 | 28.7 | 6.10E−09 | 8.6 |
| nM mAb5705 | | | | | | |
| (CD28) | | | | | | |
| mAb4241 + 2 | mAb8767P2 | mAb7221G | N.D. | 0.7 | N.D. | 1.3 |
| nM mAb5705 | | | | | | |
| (CD28) | | | | | | |
| mAb3905 + 2 | mAb17363N | mAb7195P | N.D. | 0.7 | N.D. | 1 |
| nM mAb5705 | | | | | | |
| (CD28) | | | | | | |

In addition to the antibodies discussed above in connection with Table 8, mAb5705 is an anti-CD28 antibody comprising a HCVR of SEQ ID NO: 85 and a LCVR of SEQ ID NO: 93.

### Example 5: Peptide Specificity in Jurkat/NF-κB-Luc/APC Reporter Bioassay

The engineered T cell/APC functional Jurkat/NF-κBLuc reporter/APC bioassay was also utilized to assess whether MAGE-A4×CD3 bispecific antibodies retained selectivity for the MAGE-A4 peptide over the related peptides identified in Table 7. In this assay, T2 cells were pulsed, as described previously, with the target peptide MAGE-A4 286-294 or the sequence related off-target peptides (Table 7). As summarized in Table 10, Both bsAb6043 (MAGE-A4×CD3 medium affinity) and bsAb6054 (MAGE-A4×CD3 high affinity) bispecific antibodies stimulated NF-κB-dependent reporter activity when incubated with T2 cells pulsed with the MAGE-A4 286-294 peptide. Activity was also detected when incubated with T2 cells pulsed with the off-target peptides AOX1 795-803 and SHTN1 198-206. In summary, bsAb6043 stimulated reporter activity with EC50 values of 3.0E-10 M, 3.3E-10 M, and 3.5E-09 M and S/N values of 75.2, 40.4, and 75.3 for the MAGE-A4 293-294, AOX1 795-803 and SHTN1 198-206 peptides, respectively. bsAb6054 stimulated reporter activity with EC50 values of 2.0E-12 M, 2.1E-11 M, and 1.3E-09 M and S/N values of 78.7, 36, and 81.1 for the MAGE-A4 286-294, AOX1 795-803 and SHTN1 198-206 peptides, respectively. Non-binding control bispecific antibodies with either a medium affinity CD3 arm (mAb4241) or a high affinity CD3 arm (mAb3905) were minimally active with S/N values≤ 2.9.

TABLE 10
Activity of MAGE-A4 × CD3 Bispecific Antibodies Against MAGE-A4 and
Related Peptides in Jurkat/NF-κB-Luc/T2 Pulsing Reporter Bioassay

| | bsAb6043 | | bsAb6054 | | mAb4241 | | mAb3905 | |
|---|---|---|---|---|---|---|---|---|
| | EC50 | Fold | EC50 | Fold | EC50 | Fold | EC50 | Fold |
| Cell Line | (M) | change | (M) | Change | (M) | Change | (M) | Change |
| T2 Pulsed | 3.00E−10 | 75.3 | 2.00E−12 | 78.7 | >20 nM | 1.9 | >20 nM | 1 |
| MAGE-A4 | | | | | | | | |
| 286-294 | | | | | | | | |
| T2 Pulsed | >2.0E−08 | 1 | >2.0E−08 | 1 | >2.0E−08 | 1.6 | >2.0E−08 | 1 |
| FN1 2182- | | | | | | | | |
| 2190 | | | | | | | | |
| T2 Pulsed | 3.30E−10 | 40.4 | 2.10E−11 | 36 | >20 nM | 1 | >20 nM | 1 |
| AOX1 795- | | | | | | | | |
| 803 | | | | | | | | |
| T2 Pulsed | >2.0E−08 | 1 | >2.0E−08 | 1 | >2.0E−08 | 1.4 | >2.0E−08 | 1 |
| PSME2 145- | | | | | | | | |
| 153 | | | | | | | | |
| T2 Pulsed | >2.0E−08 | 1 | >2.0E−08 | 1 | >2.0E−08 | 1.8 | >2.0E−08 | 1 |
| PCDHGC3 | | | | | | | | |
| 337-345 | | | | | | | | |
| T2 Pulsed | 3.50E−09 | 75.3 | 1.30E−09 | 81.1 | >20 nM | 1.9 | >20 nM | 1 |
| SHTN1 198- | | | | | | | | |
| 206 | | | | | | | | |
| T2 Pulsed | >2.0E−08 | 1.2 | >2.0E−08 | 1 | >2.0E−08 | 2.2 | >2.0E−08 | 1 |
| SMIM11A | | | | | | | | |
| 4-12 | | | | | | | | |
| T2 Pulsed | >2.0E−08 | 1.1 | >2.0E−08 | 1 | >2.0E−08 | 2.1 | >2.0E−08 | 1 |
| RABGAP1L | | | | | | | | |
| 408-416 | | | | | | | | |
| T2 Pulsed | >2.0E−08 | 1 | >2.0E−08 | 1 | >2.0E−08 | 2.9 | >2.0E−08 | 1 |
| CNOT1 | | | | | | | | |
| 1965-1973 | | | | | | | | |
| T2 Pulsed | >2.0E−08 | 1.2 | >2.0E−08 | 2.4 | >2.0E−08 | 1.1 | >2.0E−08 | 1 |
| MRPL37 | | | | | | | | |
| 334-342 | | | | | | | | |
| T2 Pulsed | >2.0E−08 | 1.8 | >2.0E−08 | 1.2 | >2.0E−08 | 1 | >2.0E−08 | 1 |
| CARD8 | | | | | | | | |
| 170-178 | | | | | | | | |
| T2 Pulsed | >2.0E−08 | 1.9 | >2.0E−08 | 2.8 | >2.0E−08 | 1 | >2.0E−08 | 1 |
| PLEKHH2 | | | | | | | | |
| 790-798 | | | | | | | | |
| T2 Pulsed | >2.0E−08 | 1.6 | >2.0E−08 | 1.3 | >2.0E−08 | 1 | >2.0E−08 | 1 |
| NBAS 2063- | | | | | | | | |
| 2071 | | | | | | | | |
| T2 Pulsed | >2.0E−08 | 1.9 | >2.0E−08 | 1.2 | >2.0E−08 | 1 | >2.0E−08 | 1 |
| TEP1 2156- | | | | | | | | |
| 2164 | | | | | | | | |
| T2 Pulsed | >2.0E−08 | 2.1 | >2.0E−08 | 1.2 | >2.0E−08 | 1 | >2.0E−08 | 1 |
| HEATR5A | | | | | | | | |
| 1925-1933 | | | | | | | | |
| T2 | >2.0E−08 | 1.5 | >2.0E−08 | 1.2 | >2.0E−08 | 1.1 | >2.0E−08 | 1 |
| Unpulsed | | | | | | | | |

### Example 6: Primary Image-Based Killing Assay

To assess the ability of MAGE-A4×CD3 bispecific antibodies to redirect a T cell response against cancer cells, an imaging-based multiplexed primary T cell killing assay was performed. Briefly, CD8+ T cells were isolated from human peripheral blood mononuclear cells (PBMCs) using via magnetic bead separation (Miltenyi Biotec Cat #130-045-201) following the manufacturer's protocol and the MAGE-A4 286-294 antigen presenting HLA-A*02:01 positive IM9 cell line was pre-labeled with CellTrace™ Violet (Thermo Fisher C34557) as per manufacturer instructions. IM9 multiple myeloma cells (10,000) were combined with CD8+ T cells (25,000) in 100 ul of stimulation media (X Vivo 15+10% FBS+1% HEPES+1% NaPyr+1% NEAA+0.01 mM BME) in 96 well imaging plates (Perkin Elmer Cat #6055308). Three-fold serial dilutions of antibodies ranging from 20 nM to 27.4 pM was added to the cells in an additional 100 ul of stimulation media. Following a 72-hour incubation, 50 μl of supernatant was removed for cytokine release assays (see below) and 15 μl of a 1:1000 dilution (in PBS) of the nuclear dye DRAQ5 (Thermo Fisher Cat #62252) was added to the cells. Images of DRAQ5-labeled nuclei and Cell-Trace violet labeled APCs were collected on the Opera Phenix (Perkin Elmer) and the number of viable IM9 cells in all conditions (population labeled with both CellTrace™ Violet and DRAQ5) was calculated using the Harmony analysis software (Perkin Elmer). EC50 values could not be generated from the resulting non-sigmoidal curves, so the % Viable IM9 (normalized to no antibody condition) cells was reported for the 6.6 nM dose (the highest tested dose at which the non-binding control was inactive). As summarized in Table 11, bsAb6043 (MAGE-A4×CD3 (7221G) medium affinity bispecific antibody) did not have a positive impact on the % viable IM9 cell population, but bsAb6054 (MAGE-A4×CD3 high affinity bispecific antibody) reduced the IM9 population to 38%. The addition of 2 nM of an anti-CD28 agonistic antibody doubled number of viable cells (80%) (Table 9). This can be attributed to the ability of mAb5705 to block CD28, thereby inhibiting its interaction with CD80 and CD86 which are endogenously expressed on IM9 cells. Non-binding control×CD3 bispecific antibodies mAb4241 (medium CD affinity) and mAb3905 (high CD3 affinity) had no activity in the assay.

TABLE 11
T-cell Cytotoxicity Image-Based Assay

| Treatment | % Viability at 6.6 nM | | | |
|---|---|---|---|---|
| condition | bsAb6043 | bsAb6054 | mAb4241 | mAb3905 |
| Antibody Alone | 91 | 38 | 99 | 109 |
| Antibody + | 109 | 80 | 103 | 137 |
| mAb5705 | | | | |
| (CD28) 2 nM | | | | |

### Example 7: Primary Cytokine Release Assay

IL2 and IFN-gamma release was also assessed in cell culture supernatants sampled from the 6.6 nM treatment doses utilized in the imaged based killing assay described above. Cytokine levels were determined by AlphaLisa (Perkin Elmer, Cat #AL221F, AL217F) according to manufacturer instructions and RLU values were normalized to the untreated wells to determine the S/N values. As summarized in Table 12, bsAb6043 (MAGE-A4×CD3 (7221G) medium affinity bispecific antibody) did not release any IL2 or IFN-gamma, while the higher affinity MAGE-A4×CD3 bispecific antibody bsAb6054 induced a modest production of IL2 and IFN-gamma with S/N values of 2.1 and 2.4 respectively. The addition of a CD28 antibody (mAb5705) did not significantly affect cytokine production (Table 12).

TABLE 12
IL2 and IFN-gamma Release in Three-Day Killing Assay

| | bsAb6043 | | bsAb6054 | | mAb4241 | | mAb3905 | |
|---|---|---|---|---|---|---|---|---|
| | IL2 | IFN-γ | IL2 | IFN-γ | IL2 | IFN-γ | IL2 | IFN-γ |
| Antibody | 1.2 | 0.9 | 2.1 | 2.4 | 0.7 | 0.7 | 0.6 | 0.9 |
| Alone | | | | | | | | |
| Antibody + | 0.8 | 0.8 | 2 | 1.8 | 0.8 | 0.8 | 1 | 1.1 |
| mAb5705 | | | | | | | | |
| (CD28) 2 nM | | | | | | | | |

### Example 8: Generation of MAGE-A4-Specific Chimeric Antigen Receptors

The anti-MAGE-A4 31339N2 antibodies of Table 1 were reformatted into a VL-VH single chain variable fragment (ScFv) and placed into a chimeric antigen receptor (CAR) construct that used a CD8a hinge and transmembrane domain, 4-1BB costimulatory domain, and a CD3zeta stimulatory domain, or a CD28 hinge, transmembrane, and signaling domain using the HCVR and LCVR nucleotide sequences of an anti-MAGE-A4 antibody corresponding to SEQ ID NOs: 1 and 9, respectively. The full-length nucleic acid and polypeptide heavy chain sequences of the corresponding anti-MAGE-A4 antibody (mAbH31339N2) correspond to SEQ ID NOs: 17 and 18, respectively. The full-length nucleic acid and polypeptide light chain sequences of the corresponding anti-MAGE-A4 antibody (mAbH31339N2) correspond to SEQ ID NOs: 19 and 20, respectively. The full-length nucleic acid and polypeptide HLA-A2/MAGE-A₄₂₈₆₋₂₉₄-targeted CAR sequences correspond to SEQ ID NOs: 21 and 22, respectively. As a non-binding control, a similar CAR was designed using the nucleotide sequence of an irrelevant scFv. The MAGE-A4-specific CAR was cloned into a lentiviral expression vector (Lenti-X™ Bicistronic Expression System (Neo), Clontech Cat #632181) and lentiviral particles were generated via the Lenti-X Packaging Single-Shot (VSV-G) system (Clontech Cat #631276) according to manufacturer protocols. Jurkat cells engineered to express an NFKB-luciferase reporter (Jurkat/NKFBLuc cl 1C11) were then transduced with the CAR construct using RetroNectin® Precoated Dishes (Clontech, Cat #T110a) according to manufacturer's protocols. Following selection for at least 2 weeks in 500 μg/ml G418 (Gibco, Cat #11811-098), the following CAR T cell line was generated; Jurkat/NKFBLuc cl 1C11/MAGE-A4 (286-294) 31339 VL-VH CART. As a non-binding control, a similar CAR was designed using the nucleotide sequence of an irrelevant scFv. This CAR T cell line was evaluated for cell surface expression and functional activity in response to MAGE-A4 expressing cells.

### Example 9: Cell Surface Expression of MAGE-A4 CAR Constructs in Jurkat Cells and Activation of MAGE-A4 CAR T Cells

The relative level of cell surface expression of the MAGE-A4 CAR construct in Jurkat/NF-κB-Luc cells was assessed by flow cytometry. To stain, cells were plated in a staining buffer of PBS without calcium and magnesium (Irving 9240), and 2% BSA (Sigma-Aldrich, Cat #A8577) at a density of 200,000 cells per well in a 96 well V-Bottom plate and stained for 30 mins at 4° C. with 10 μg/ml of Protein L (Genscript Biotin Protein L, Cat #M00097). Following incubation, cells were washed once in staining buffer, and stained with streptavidin Alexa-647 secondary antibody (Biolegend, Cat #405237) at 0.5 μg/ml for 30 mins at 4° C. Finally, cells were stained with a green fluorescent viability dye (Molecular Probes, Cat #L-34970, reconstituted in 50 μL DMSO) at a concentration of 1:1000. Cells were then washed and fixed using a 50% solution of BD Cytofix (Becton Dickinson, Cat #554655) diluted in PBS. Samples were run on the Intellicyt iQue flow cytometer and analyzed by FlowJo 10.2 after gating for live cells to calculate the mean fluorescent intensity (MFI). The percent of Protein L positive cells was calculated by taking the number of protein L positive cells and dividing by the total number of cells. Table 13 shows that the Jurkat/NF-κB-Luc/MAGE-A4 31339N2 VL-VH CAR-T cell line was 33.8% CAR positive, as measured by percent protein L staining. The non-targeting control CAR was expressed in 67.2% of cells.

Activity of the CAR T cell lines was assessed in a CAR T/APC (antigen presenting cell) bioassay. To perform the bioassay, 50,000 CAR T cells were added to Thermo-Nunc 96-well white plates (Thermo Fisher Scientific, Cat #136101) in 50 μl of assay media (RPMI media with 10% FBS and 1% P/S/G) followed by the addition of a 3-fold serial dilution of APCs (500,000 cells to 685 cells) in 50 μl of assay media. The following APCs were utilized: IM9 (which endogenously express the MAGE-A4 286-294 peptide and are HLA-A*02:01 positive), and HEK293 (which are MAGE-A4 286-294 negative but HLA-A*02:01 positive). The cell mixtures were incubated in a 37° C., 5% CO₂, humidified incubator for 5 hours. NF-κB-Luciferase activity was measured using Promega One-Glo (Cat #E6130) and a Perkin Elmer Envision plate reader. Relative luciferase units (RLU) were generated and plotted in GraphPad Prism using a four-parameter logistic equation over an 8-point dose response curve to generate EC50 values (number of APC). The zero APC condition for each dose-response curve was also included in the analysis as a continuation of the three-fold serial dilution and represented as the lowest dose. CAR-T activity was determined and reported as the EC50 (number of APCs) and by taking the ratio of the highest RLU on the curve to the lowest and is represented as signal:noise (S:N) in Table 14. The 31339N2 CAR-T was activated upon incubation with IM9 cells with an EC50 of 14, 669 cells and a S/N of 37.4, while the non-targeting control CAR-T was only activated 4.9-fold. No activation was observed on HEK293 cells (which are MAGE-A4 negative but HLA-A2 positive).

TABLE 13
Protein L staining of CARs in Jurkat/NFKBLuc cl 1C11 cells

| | % Protein L |
|---|---|
| Cell Line | positive |
| Jurkat/NFKBLuc cl. 1C11 | 3.9 |
| Jurkat/NFKBLuc cl. 1C11/MAGE-A4 (286-294) | 33.8 |
| 31339N2 VL-VH CAR T-cells | |
| Jurkat/NFKBLuc cl. 1C11/Control VL-VH CAR T-cells | 67.2 |

TABLE 14
Activation of MAGE-A4 CAR T cells
in a CAR T cell/APC Bioassay

| | |
|---|---|
| IM9 APC | HEK293 APC |

| CAR Cell Line | EC50 | MAX S:N | EC50 | MAX S:N |
|---|---|---|---|---|
| Parental Cell | N.D. | 5.2 | N.D. | 1 |
| 31339N2 | 14,669 | 37.4 | N.D. | 0.9 |
| Control CAR | N.D. | 4.9 | N.D. | 0.8 |

### Example 10: Binding of Anti-HLA-A2:MAGE-A4 230-239 Antibodies Via Flow Cytometry to T2 Cells Pulsed with MAGE-A4 230-239 and Related Off-Target Peptides

Cell surface binding of the anti-HLA-A2: MAGE-A4 230-239 antibody (mAbM34852N) to HLA-A*02:01 positive T2 (174 CEM.T2) cells was assessed in a flow cytometry-based peptide pulsing assay. To pulse, 1×10⁶ T2 cells were incubated with 10 μg/ml of human (h) B2M (EMD Millipore Catalog No. 475828) and 100 μg/ml of a MAGE-A4 230-239 peptide in 1 ml of AIM V medium (Gibco Catalog No. 31035-025) for 16 h at 37° ° C. Cells were washed in staining buffer (PBS without calcium and magnesium (Irvine Scientific Catalog No. 9319)+2% FBS (Seradigm, Lot No. 238B15), harvested using cell dissociation buffer (Millipore, Catalog No. S-004-C), and resuspended in staining buffer. Pulsed Cells (200,000) were plated in 96 well V-Bottom plates (Axygen, Catalog No. P-96-450V-C-S) and stained with three-fold serial dilutions (1.7 pM-100 nM) of mAbM34852N or a non-binding control antibody (mAb1097) for 30 mins at 4° C. Cells were then washed once in staining buffer, and incubated with an Alexa Fluor 647 conjugated Fab′2 anti-mouse Fc-specific secondary antibody (Jackson ImmunoResearch, Catalog No. 115-606-071) at 5 μg/ml for 30 mins at 4° C. Finally, cells were stained with a green fluorescent viability dye (Molecular Probes Catalog No. L-34970), reconstituted in 50 μl DMSO at a concentration of 1:1000. Cells were then washed and fixed using a 50% solution of BD Cytofix (BD, Catalog No. 554655) diluted in PBS. Samples were run on an IntelliCyt iQue flow cytometer (Intellicyt) and results were analyzed using Forecyte analysis software (Intellicyt) to calculate the mean fluorescent intensity (MFI) after gating for live cells. MFI values were plotted in Graphpad Prism using a four-parameter logistic equation over a 12-point response curve to calculate EC50 values. The secondary antibody alone (i.e., no primary antibody) for each dose-response curve was also included in the analysis as a continuation of the three-fold serial dilution and was represented as the lowest dose. The signal to noise (S/N) was determined by taking the ratio of the highest MFI on the dose response curve to the MFI in the secondary-alone wells. The EC50 values (M) and max S/N are shown in Table 15. mAbM34852N bound with an EC50 of 4.7 nM and a max S/N of 243.3, whereas binding of the control antibody (mAb1097) was only weakly detected.

TABLE 15
Binding of Anti-HLA-A2:MAGE-A4 (230-239) Antibody to T2
cells Pulsed with MAGE-A4 (230-239) Via Flow Cytometry

| mAb | EC₅₀ (M) | Max S/N |
|---|---|---|
| mAbM34852N | 4.7E−09 | 243.3 |
| mAb1097 (Isotype | ND | 13.4 |
| Control) | | |

ND = EC50 values could not be determined with accuracy because the binding did not reach saturation within the tested antibody concentration range

An in-silico computational strategy identified several MAGE-A4-related peptides predicted to form complexes with HLA-A*02:01. The identified peptides are summarized in Table 16. Binding of the HLA-A2: MAGE-A4 230-239 antibody (mAbM34852N), and a non-binding isotype control antibody (mAb1097) to these related peptides was assessed at a single concentration of 100 nM in the T2 pulsing assay described above. Binding here is displayed in Table 17 as an MFI ratio of the binding to peptide pulsed cell divided by the binding to unpulsed cells. mAbM34852N bound the MAGE-A4 peptide with a binding ratio of 512.9 while the isotype control mAb 1097 bound it with a ratio of 4.3. mAbM34852N had similar binding to a MAGE-A8 (232-241) peptide with a binding ratio of 870.2; however, no detectable binding to the remaining peptides was observed and the control antibody binding was <4.3 for all tested peptides, indicating that mAbM34852N has potential as a therapeutic against MAGE-A4.

TABLE 16
Sequences of MAGE-A4 (230-239) Peptide and
Predicted Off-Target Peptides

| Peptide (Gene name + | | |
|---|---|---|
| location within full-length | Peptide | |
| protein) | Sequence | SEQ ID NO. |
| MAGE-A4 (230-239) | GVYDGREHTV | 122 |
| MAGE-A8 (232-241) | GLYDGREHSV | 123 |
| THBS3 (116-125) | GLADGRTHTV | 124 |
| LPIN2 (823-832) | GVPDCRIFTV | 125 |
| SCL25A19 (111-120) | SVYDAREFSV | 126 |
| CELSR3 (1594-1603) | GLSDGQWHTV | 127 |
| PAPPA2 (307-316) | GVFDNCSHTV | 128 |
| FAT4 (4053-4062) | KVSDGHFHTV | 129 |
| MAGE-A10 (254-263) | GLYDGMEHLI | 130 |
| CCDC36 (417-426) | FLCDPREHLV | 131 |

Changes from the MAGE-A4 (230-239) sequence are underlined

TABLE 17
Binding of Anti-HLA-A2:MAGE-A4 (230-239) Antibody to T2
cells Pulsed with Related Peptides Via Flow Cytometry

| | |
|---|---|
| | Antibody Binding Ratio (pulsed |
| Peptide (Gene name + | cell MFI/Unpulsed cell MFI |

| peptide location) | mAbM34852N | mAb1097 |
|---|---|---|
| MAGE-A4 (230-239) | 512.9 | 4.3 |
| MAGE-A8 (232-241) | 870.2 | 3.3 |
| THBS3 (116-125) | 1.0 | 1.0 |
| LPIN2 (823-832) | 0.9 | 0.9 |
| SCL25A19 (111-120) | 0.8 | 0.9 |
| CELSR3 (1594-1603) | 0.8 | 0.9 |
| PAPPA2 (307-316) | 0.9 | 0.9 |
| FAT4 (4053-4062) | 1.0 | 1.1 |
| MAGE-A10 (254-263) | 0.9 | 1.3 |
| CCDC36 (417-426) | 1.0 | 1.1 |

### Example 11: HLA-A2: MAGE-A4 (230-239) Alanine Scanning

Cell surface binding of the anti-HLA-A2: MAGE-A4 230-239 antibody (mAbM34852N) to HLA-A*02:01 positive T2 (174 CEM.T2) cells was assessed in a flow cytometry-based peptide pulsing assay. To pulse, 1×10⁶ T2 cells were incubated with 10 μg/ml of human (h) B2M (EMD Millipore Cat. No. 475828) and 100 μg/ml of a MAGE-A4 230-239 peptide in 1 ml of AIM V medium (Gibco Cat. No. 31035-025) for 16 h at 37° ° C. Cells were washed in staining buffer (PBS without calcium and magnesium (Irvine Scientific Cat. No. 9319)+2% FBS (Seradigm, Lot No. 238B15)), harvested using cell dissociation buffer (Millipore, Cat. No. S-004-C), and resuspended in staining buffer. Pulsed Cells (200,000) were plated in 96 well V-Bottom plates (Axygen, Cat. No. P-96-450V-C-S) and stained with three-fold serial dilutions (1.7 pM-100 nM) of mAbM34852N or a non-binding isotype control antibody for 30 mins at 4° C. Cells were then washed once in staining buffer, and incubated with an Alexa Fluor 647 conjugated Fab′2 anti-mouse Fc-specific secondary antibody (Jackson ImmunoResearch, Cat. No. 115-606-071) at 5 μg/ml for 30 mins at 4° C. Finally, cells were stained with a green fluorescent viability dye (Molecular Probes Cat. No. L-34970), reconstituted in 50 μl DMSO at a concentration of 1:1000. Cells were then washed and fixed using a 50% solution of BD Cytofix (BD, Cat. No. 554655) diluted in PBS. Samples were run on an IntelliCyt iQue flow cytometer (Intellicyt) and results were analyzed using Forecyte analysis software (Intellicyt) to calculate the mean fluorescent intensity (MFI) after gating for live cells. MFI values were plotted in Graphpad Prism using a four-parameter logistic equation over a 12-point response curve to calculate EC₅₀ values. The secondary antibody alone (i.e., no primary antibody) for each dose-response curve was also included in the analysis as a continuation of the three-fold serial dilution and was represented as the lowest dose. The signal to noise (S/N) was determined by taking the ratio of the highest MFI on the dose response curve to the MFI in the secondary-alone wells. The EC₅₀ values (M) and max S/N are shown in Table 19. mAbM34852N bound with an EC₅₀ of 4.7 nM and a max S/N of 243.3, whereas binding of the control antibody was only weakly detected. The results from the alanine scanning assay show that mAbM34852 exhibited binding across the MAGE-A4 (230-239) peptide.

TABLE 18
Sequences of MAGE-A4 (230-239) Peptide
Alanine Scanning Peptides

| Peptide (gene name + | | SEQ |
|---|---|---|
| peptide location) | Peptide Sequence | ID NO. |
| MAGE-A4 (230-239) | GVYDGREHTV | 122 |
| MAGE-A4 (230-239) G230A | AVYDGREHTV | 132 |
| MAGE-A4 (230-239) V231A | GAYDGREHTV | 133 |
| MAGE-A4 (230-239) Y232A | GVADGREHTV | 134 |
| MAGE-A4 (230-239) D233A | GVYAGREHTV | 135 |
| MAGE-A4 (230-239) G234A | GVYDAREHTV | 136 |
| MAGE-A4 (230-239) R235A | GVYDGAEHTV | 137 |
| MAGE-A4 (230-239) E236A | GVYDGRAHTV | 138 |
| MAGE-A4 (230-239) H237A | GVYDGREATV | 139 |
| MAGE-A4 (230-239) T238A | GVYDGREHAV | 140 |
| MAGE-A4 (230-239) V239A | GVYDGREHTA | 141 |

TABLE 19
Binding of Anti-HLA-A2:MAGE-A4 (230-239) Antibody to T2 cells
Pulsed with Alanine Scanning peptides Via Flow Cytometry

Antibody Binding Ratio (pulsed
cell MFI/Unpulsed cell MFI

| Peptide (gene name + | | Isotype |
|---|---|---|
| peptide location) | mAbM34852N | control |
| MAGE-A4 (230-239) | 121.3 | 1 |
| MAGE-A4 (230-239) G230A | 38.0 | 0.5 |
| MAGE-A4 (230-239) V231A | 35.5 | 0.5 |
| MAGE-A4 (230-239) Y232A | 1.0 | 0.5 |
| MAGE-A4 (230-239) D233A | 1.4 | 0.5 |
| MAGE-A4 (230-239) G234A | 22.9 | 0.5 |
| MAGE-A4 (230-239) R235A | 4.0 | 0.5 |
| MAGE-A4 (230-239) E236A | 42.6 | 0.5 |
| MAGE-A4 (230-239) H237A | 82.0 | 0.5 |
| MAGE-A4 (230-239) T238A | 108.6 | 0.5 |
| MAGE-A4 (230-239) V239A | 80.0 | 0.5 |

### DESCRIPTION OF SEQUENCES

TABLE 20
Sequence Identifiers

| SEQ ID NO. | DNA/Polypeptide | Sequence |
|---|---|---|
| 1 | DNA | mAbM31339N2 and |
| | | mAbH31339N2 HCVR |
| 2 | Polypeptide | mAbM31339N2 and |
| | | mAbH31339N2 HCVR |
| 3 | DNA | mAbM31339N, |
| | | mAbM31339N2 and |
| | | mAbH31339N2 HCDR1 |
| 4 | Polypeptide | mAbM31339N, |
| | | mAbM31339N2 and |
| | | mAbH31339N2 HCDR1 |
| 5 | DNA | mAbM31339N, |
| | | mAbM31339N2 and |
| | | mAbH31339N2 HCDR2 |
| 6 | Polypeptide | mAbM31339N, |
| | | mAbM31339N2 and |
| | | mAbH31339N2 HCDR2 |
| 7 | DNA | mAbM31339N, |
| | | mAbM31339N2 and |
| | | mAbH31339N2 HCDR3 |
| 8 | Polypeptide | mAbM31339N, |
| | | mAbM31339N2 and |
| | | mAbH31339N2 HCDR3 |
| 9 | DNA | mAbM31339N, |
| | | mAbM31339N2 and |
| | | mAbH31339N2 LCVR |
| 10 | Polypeptide | mAbM31339N, |
| | | mAbM31339N2 and |
| | | mAbH31339N2 LCVR |
| 11 | DNA | mAbM31339N, |
| | | mAbM31339N2 and |
| | | mAbH31339N2 LCDR1 |
| 12 | Polypeptide | mAbM31339N, |
| | | mAbM31339N2 and |
| | | mAbH31339N2 LCDR1 |
| 13 | DNA | mAbM31339N, |
| | | mAbM31339N2 and |
| | | mAbH31339N2 LCDR2; and |
| | | anti-CD3-A (mAbH7221G) |
| | | LCDR2; and anti-CD3-B |
| | | (mAbH7195P) LCDR2; and |
| | | mAbM34852N LCDR2 |
| 14 | Polypeptide | mAbM31339N, |
| | | mAbM31339N2 and |
| | | mAbH31339N2 LCDR2; and |
| | | anti-CD3-A (mAbH7221G) |
| | | LCDR2; and anti-CD3-B |
| | | (mAbH7195P) LCDR2; and |
| | | mAbM34852N LCDR2 |
| 15 | DNA | mAbM31339N, |
| | | mAbM31339N2 and |
| | | mAbH31339N2 LCDR3 |
| 16 | Polypeptide | mAbM31339N, |
| | | mAbM31339N2 and |
| | | mAbH31339N2 LCDR3 |
| 17 | DNA | mAbH31339N2HC |
| 18 | Polypeptide | mAbH31339N2HC |
| 19 | DNA | mAbH31339N2LC |
| 20 | Polypeptide | mAbH31339N2LC |
| 21 | DNA | Full length 31339N2 HLA- |
| | | A2/MAGE-A4₂₈₆₋₂₉₄-targeted |
| | | CAR (4-1BB costimulatory |
| | | domain) |
| 22 | Polypeptide | Full length 31339N2 HLA- |
| | | A2/MAGE-A4₂₈₆₋₂₉₄-targeted |
| | | CAR (4-1BB costimulatory |
| | | domain) |
| 23 | Polypeptide | (G4S)₁ linker |
| 24 | Polypeptide | (G4S)₂ linker |
| 25 | Polypeptide | (G4S)₃ linker |
| 26 | Polypeptide | GSTSGSGKPGSGEGSTKG |
| | | linker |
| 27 | Polypeptide | CD8α hinge |
| 28 | Polypeptide | CD8 Transmembrane domain |
| 29 | Polypeptide | 4-1BB costimulatory domain |
| 30 | Polypeptide | CD3zeta signaling domain |
| 31 | DNA | Full length MAGE-A4 |
| 32 | Polypeptide | Full length MAGE-A4 |
| 33 | Polypeptide | MAGE-A4 (286-294) |
| 34 | Polypeptide | CD28 hinge |
| 35 | DNA | CD28 hinge |
| 36 | Polypeptide | CD28 transmembrane |
| 37 | DNA | CD28 transmembrane |
| 38 | Polypeptide | CD28 costimulatory domain |
| 39 | DNA | CD28 costimulatory domain |
| 40 | Polypeptide | FN1 2182-2190 |
| 41 | Polypeptide | AOX1 795-803 |
| 42 | Polypeptide | PSME2 145-153 |
| 43 | Polypeptide | PCDHGC3 337-345 |
| 44 | Polypeptide | SHTN1 198-206 |
| 45 | Polypeptide | SMIM11A 4-12 |
| 46 | Polypeptide | RABGAP1L 408-416 |
| 47 | Polypeptide | CNOT1 1965-1973 |
| 48 | Polypeptide | CARD8 170-178 |
| 49 | Polypeptide | MRPL37 334-342 |
| 50 | Polypeptide | PLEKHH2 790-798 |
| 51 | Polypeptide | NBAS 2063-2071 |
| 52 | Polypeptide | TEP1 2156-2164 |
| 53 | Polypeptide | HEATR5A 1925-1933 |
| 54 | DNA | anti-CD3-A (mAbH7221G) |
| | | HCVR |
| 55 | Polypeptide | anti-CD3-A (mAbH7221G) |
| | | HCVR |
| 56 | DNA | anti-CD3-A (mAbH7221G) |
| | | HCDR1 |
| 57 | Polypeptide | anti-CD3-A (mAbH7221G) |
| | | HCDR1 |
| 58 | DNA | anti-CD3-A (mAbH7221G) |
| | | HCDR2 |
| 59 | Polypeptide | anti-CD3-A (mAbH7221G) |
| | | HCDR2 |
| 60 | DNA | anti-CD3-A (mAbH7221G) |
| | | HCDR3 |
| 61 | Polypeptide | anti-CD3-A (mAbH7221G) |
| | | HCDR3 |
| 62 | DNA | anti-CD3-A (mAbH7221G) |
| | | LCVR; and anti-CD3-B |
| | | (mAbH7195P) LCVR |
| 63 | Polypeptide | anti-CD3-A (mAbH7221G) |
| | | LCVR; and anti-CD3-B |
| | | (mAbH7195P) LCVR |
| 64 | DNA | anti-CD3-A (mAbH7221G) |
| | | LCDR1; and anti-CD3-B |
| | | (mAbH7195P) LCDR1 |
| 65 | Polypeptide | anti-CD3-A (mAbH7221G) |
| | | LCDR1; and anti-CD3-B |
| | | (mAbH7195P) LCDR1 |
| 66 | DNA | anti-CD3-A (mAbH7221G) |
| | | LCDR3; and anti-CD3-B |
| | | (mAbH7195P) LCDR3 |
| 67 | Polypeptide | anti-CD3-A (mAbH7221G) |
| | | LCDR3; and anti-CD3-B |
| | | (mAbH7195P) LCDR3 |
| 68 | DNA | anti-CD3-A (mAbH7221G) |
| | | HC |
| 69 | Polypeptide | anti-CD3-A (mAbH7221G) |
| | | HC |
| 70 | DNA | anti-CD3-A (mAbH7221G) |
| | | LC; and anti-CD3-B |
| | | (mAbH7195P) LC |
| 71 | Polypeptide | anti-CD3-A (mAbH7221G) |
| | | LC; and anti-CD3-B |
| | | (mAbH7195P) LC |
| 72 | DNA | anti-CD3-B (mAbH7195P) |
| | | HCVR |
| 73 | Polypeptide | anti-CD3-B (mAbH7195P) |
| | | HCVR |
| 74 | DNA | anti-CD3-B (mAbH7195P) |
| | | HCDR1 |
| 75 | Polypeptide | anti-CD3-B (mAbH7195P) |
| | | HCDR1 |
| 76 | DNA | anti-CD3-B (mAbH7195P) |
| | | HCDR2 |
| 77 | Polypeptide | anti-CD3-B (mAbH7195P) |
| | | HCDR2 |
| 78 | DNA | anti-CD3-B (mAbH7195P) |
| | | HCDR3 |
| 79 | Polypeptide | anti-CD3-B (mAbH7195P) |
| | | HCDR3 |
| 80 | DNA | anti-CD3-B (mAbH7195P) HC |
| 81 | Polypeptide | anti-CD3-B (mAbH7195P) HC |
| 82 | DNA | mAbM31339N HCVR |
| 83 | Polypeptide | mAbM31339N HCVR |
| 84 | DNA | CD28 HCVR |
| 85 | Polypeptide | CD28 HCVR |
| 86 | DNA | CD28 HCDR1 |
| 87 | Polypeptide | CD28 HCDR1 |
| 88 | DNA | CD28 HCDR2 |
| 89 | Polypeptide | CD28 HCDR2 |
| 90 | DNA | CD28 HCDR3 |
| 91 | Polypeptide | CD28 HCDR3 |
| 92 | DNA | CD28 LCVR |
| 93 | Polypeptide | CD28 LCVR |
| 94 | DNA | CD28 LCDR1 |
| 95 | Polypeptide | CD28 LCDR1 |
| 96 | DNA | CD28 LCDR2 |
| 97 | Polypeptide | CD28 LCDR2 |
| 98 | DNA | CD28 LCDR3 |
| 99 | Polypeptide | CD28 LCDR3 |
| 100 | DNA | CD28 HC |
| 101 | Polypeptide | CD28 HC |
| 102 | DNA | CD28 LC |
| 103 | Polypeptide | CD28 LC |
| 104 | DNA | Full length 31339N2 HLA- |
| | | A2/MAGE-A4₂₈₆₋₂₉₄-targeted |
| | | CAR (CD28 costimulatory |
| | | domain) |
| 105 | Polypeptide | Full length 31339N2 HLA- |
| | | A2/MAGE-A4₂₈₆₋₂₉₄-targeted |
| | | CAR (CD28 costimulatory |
| | | domain) |
| 106 | DNA | mAbM34852N HCVR |
| 107 | Polypeptide | mAbM34852N HCVR |
| 108 | DNA | mAbM34852N HCDR1 |
| 109 | Polypeptide | mAbM34852N HCDR1 |
| 110 | DNA | mAbM34852N HCDR2 |
| 111 | Polypeptide | mAbM34852N HCDR2 |
| 112 | DNA | mAbM34852N HCDR3 |
| 113 | Polypeptide | mAbM34852N HCDR3 |
| 114 | DNA | mAbM34852N LCVR |
| 115 | Polypeptide | mAbM34852N LCVR |
| 116 | DNA | mAbM34852N LCDR1 |
| 117 | Polypeptide | mAbM34852N LCDR1 |
| 118 | DNA | mAbM34852N LCDR3 |
| 119 | Polypeptide | mAbM34852N LCDR3 |
| 120 | Polypeptide | Full length 31339N2 HLA- |
| | | A2/MAGE-A4₂₈₆₋₂₉₄-targeted |
| | | CAR (4-1BB costimulatory |
| | | domain) |
| 121 | Polypeptide | Full length 31339N2 HLA- |
| | | A2/MAGE-A4₂₈₆₋₂₉₄-targeted |
| | | CAR (CD28 costimulatory |
| | | domain) |
| 122 | Polypeptide | MAGE-A4 (230-239) |
| 123 | Polypeptide | MAGE-A8 (232-241) |
| 124 | Polypeptide | THBS3 (116-125) |
| 125 | Polypeptide | LPIN2 (823-832) |
| 126 | Polypeptide | SCL25A19 (111-120) |
| 127 | Polypeptide | CELSR3 (1594-1603) |
| 128 | Polypeptide | PAPPA2 (307-316) |
| 129 | Polypeptide | FAT4 (4053-4062) |
| 130 | Polypeptide | MAGE-A10 (254-263) |
| 131 | Polypeptide | CCDC36 (417-426) |
| 132 | Polypeptide | MAGE-A4 (230-239) G230A |
| 133 | Polypeptide | MAGE-A4 (230-239) V231A |
| 134 | Polypeptide | MAGE-A4 (230-239) Y232A |
| 135 | Polypeptide | MAGE-A4 (230-239) D233A |
| 136 | Polypeptide | MAGE-A4 (230-239) G234A |
| 137 | Polypeptide | MAGE-A4 (230-239) R235A |
| 138 | Polypeptide | MAGE-A4 (230-239) E236A |
| 139 | Polypeptide | MAGE-A4 (230-239) H237A |
| 140 | Polypeptide | MAGE-A4 (230-239) T238A |
| 141 | Polypeptide | MAGE-A4 (230-239) V239A |

### ANNOTATED SEQUENCES

In the following annotated sequences, the parts are identified by alternating non-underlined sections with underlined sections, and the order of the parts corresponds to the order listed below each sequence (i.e., the first non-underlined section is the VL, the following underlined section is the (G4S)3, the following non-underlined section is the VH, and so on).

MAGE-A4(286-294) 31339N2 VL-VH BBz CAR
(SEQ ID NO: 22)
DIQMTQSPSSLSASVGDRVTITCRASQSIDTFLNWYQQKPGKVPKLLIYAASNLESGVPSRFS
GSGSGTVFTLTISRLQPEDFATYYCQQSYSIPPITFGQGTRVEIKRGGGGSGGGGSGGGGSQV
QLVESGGGLVKPGGSLRLSCAASGFTFSEYYLTWIRQAPGKGLDWIAYISSSGYNIYYADSV
KDRFTISRDNGKNSLFLQMNNLRAEDTAVYFCAREGVTDGMDVWGQGTTVTVSSGGGGS
TTTPAPRPPTPAPTIASQPLSLRPEACRPAAGGAVHTRGLDFACDIYIWAPLAGTCGVLLLSL
VITLYCKRGRKKLLYIFKQPFMRPVQTTQEEDGCSCRFPEEEEGGCELRVKFSRSADAPAYK
QGQNQLYNELNLGRREEYDVLDKRRGRDPEMGGKPRRKNPQEGLYNELQKDKMAEAYSE
IGMKGERRRGKGHDGLYQGLSTATKDTYDALHMQALPPR
VL
(G4S)3
VH
G4S
CD8 hinge/TM
4-1BB costimulatory domain
CD3Z
MAGE-A4(286-294) 31339N2 VL-VH BBz CAR
(SEQ ID NO: 120)
DIQMTQSPSSLSASVGDRVTITCRASQSIDTFLNWYQQKPGKVPKLLIYAASNLESGVPSRFS
GSGSGTVFTLTISRLQPEDFATYYCQQSYSIPPITFGQGTRVEIKGGGGSGGGGSGGGGSQVQ
LVESGGGLVKPGGSLRLSCAASGFTFSEYYLTWIRQAPGKGLDWIAYISSSGYNIYYADSVK
DRFTISRDNGKNSLFLQMNNLRAEDTAVYFCAREGVTDGMDVWGQGTTVTVSSGGGGSTT
TPAPRPPTPAPTIASQPLSLRPEACRPAAGGAVHTRGLDFACDIYIWAPLAGTCGVLLLSLVI
TLYCKRGRKKLLYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
VL
(G4S)3
VH
G4S
CD8 hinge/TM
4-1BB costimulatory domain
CD3Z
MAGE-A4(286-294) 31339N2 VL-VH CD28hinge/TM/cytoCD3z CAR
(SEQ ID NO: 105)
DIQMTQSPSSLSASVGDRVTITCRASQSIDTFLNWYQQKPGKVPKLLIYAASNLESGVPSRFS
GSGSGTVFTLTISRLQPEDFATYYCQQSYSIPPITFGQGTRVEIKRGGGGSGGGGSGGGGSQV
QLVESGGGLVKPGGSLRLSCAASGFTFSEYYLTWIRQAPGKGLDWIAYISSSGYNIYYADSV
KDRFTISRDNGKNSLFLQMNNLRAEDTAVYFCAREGVTDGMDVWGQGTTVTVSSGGGGSI
EVMYPPPYLDNEKSNGTIIHVKGKHLCPSPLFPGPSKPFWVLVVVGGVLACYSLLVTVAFIIF
WVRSKRSRLLHSDYMNMTPRRPGPTRKHYQPYAPPRDFAAYRSRVKFSRSADAPAYKQGQ
NQLYNELNLGRREEYDVLDKRRGRDPEMGGKPRRKNPQEGLYNELQKDKMAEAYSEIGM
KGERRRGKGHDGLYQGLSTATKDTYDALHMQALPPR
VL
(G4S)3
no
VH
G4S
CD28 hinge
CD28 TM
CD28 costimulatory domain
CD3Z
MAGE-A4(286-294) 31339N2 VL-VH CD28hinge/TM/cytoCD3z CAR
(SEQ ID NO: 121)
DIQMTQSPSSLSASVGDRVTITCRASQSIDTFLNWYQQKPGKVPKLLIYAASNLESGVPSRFS
GSGSGTVFTLTISRLQPEDFATYYCQQSYSIPPITFGQGTRVEIKGGGGSGGGGGGGGSQVQ
LVESGGGLVKPGGSLRLSCAASGFTFSEYYLTWIRQAPGKGLDWIAYISSSGYNIYYADSVK
DRFTISRDNGKNSLFLQMNNLRAEDTAVYFCAREGVTDGMDVWGQGTTVTVSSGGGGSIE
VMYPPPYLDNEKSNGTIIHVKGKHLCPSPLFPGPSKPFWVLVVVGGVLACYSLLVTVAFIIF
WVRSKRSRLLHSDYMNMTPRRPGPTRKHYQPYAPPRDFAAYRSRVKFSRSADAPAYKQGQ
NQLYNELNLGRREEYDVLDKRRGRDPEMGGKPRRKNPQEGLYNELQKDKMAEAYSEIGM
KGERRRGKGHDGLYQGLSTATKDTYDALHMQALPPR
VL
(G4S)3
VH
G4S
CD28 hinge
CD28 TM
CD28 costimulatory domain
CD3Z

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. An antigen-binding protein that specifically binds an HLA-bound Melanoma-Associated Antigen A4 (MAGE-A4), wherein the antigen-binding protein comprises a light chain variable region (LCVR) and a heavy chain variable region (HCVR), wherein the LCVR comprises complementarity determining regions (CDRs) of a LCVR comprising the amino acid sequence of SEQ ID NO: 10 or SEQ ID NO: 115, and wherein the HCVR comprises CDRs of a HCVR comprising the amino acid sequence of SEQ ID NO: 2, SEQ ID NO: 83, or SEQ ID NO: 107.

2. The antigen-binding protein of claim 1, wherein the LCVR comprises an amino acid sequence having at least 95% sequence identity to SEQ ID NO: 10 or SEQ ID NO: 115.

3. The antigen-binding protein of claim 1 or claim 2, wherein the HCVR comprises an amino acid sequence having at least 95% identity to SEQ ID NO: 2, SEQ ID NO: 83, or SEQ ID NO: 107.

4. The antigen-binding protein of any one of claims 1-3, wherein said MAGE-A4-specific antigen-binding protein interacts with amino acids 286-294, or a portion thereof, of SEQ ID NO: 32.

5. A MAGE-A4-specific chimeric antigen receptor (CAR) comprising from N-terminus to C-terminus: (a) an extracellular ligand-binding domain comprising an anti-MAGE-A4 single chain variable fragment (scFv) domain comprising a light chain variable region (LCVR) and a heavy chain variable region (HCVR); (b) a hinge; (c) a transmembrane domain; and (d) a cytoplasmic domain comprising a 4-1BB costimulatory domain or a CD28 costimulatory domain and a CD3zeta signaling domain, wherein the LCVR comprises complementarity determining regions (CDRs) of a LCVR comprising the amino acid sequence of SEQ ID NO: 10 or SEQ ID NO: 115, and CDRs of a HCVR comprising an amino acid sequence of SEQ ID NO: 2, SEQ ID NO: 83 or SEQ ID NO: 107.

6. The MAGE-A4 specific CAR of claim 5, wherein said MAGE-A4-specific CAR comprises, from N-terminus to C-terminus: (a) the extracellular ligand-binding domain; (b) a hinge; (c) a transmembrane domain; and (d) a cytoplasmic domain comprising a costimulatory domain and a signaling domain.

7. The MAGE-A4-specific CAR of claim 5, wherein the anti-MAGE-A4 scFv domain comprises a first linker between the LCVR and the HCVR.

8. The MAGE-A4-specific CAR of any one of claims 5-7, further comprising a second linker between the extracellular ligand-binding domain and the hinge.

9. The MAGE-A4-specific CAR of claim 8, wherein the first linker comprises an amino acid sequence selected from the group consisting of SEQ ID NOs: 23-26, and wherein the second linker comprises an amino acid sequence selected from the group consisting of SEQ ID NOs: 23-26.

10. The MAGE-A4-specific CAR of claim 9, wherein the first linker comprises the amino acid sequence of SEQ ID NO: 25 and the second linker comprises the amino acid sequence of SEQ ID NO: 23.

11. The MAGE-A4-specific CAR of any one of claims 5-10, wherein the hinge, the transmembrane domain, or both, are from a CD8a polypeptide.

12. The MAGE-A4-specific CAR of any one of claims 5-11, wherein the costimulatory domain comprises a 4-1BB costimulatory domain.

13. The MAGE-A4-specific CAR of any one of claims 5-11, wherein the costimulatory domain comprises a CD28 costimulatory domain.

14. The MAGE-A4-specific CAR of any one of claims 5-10, 12 and 13, wherein the hinge, the transmembrane domain, or both, are from a CD28 polypeptide.

15. The MAGE-A4-specific CAR of any one of claims 5-12, wherein the hinge comprises the amino acid sequence of SEQ ID NO: 27.

16. The MAGE-A4-specific CAR of any one of claims 5-12, wherein the transmembrane domain comprises the amino acid sequence of SEQ ID NO: 28.

17. The MAGE-A4-specific CAR of any one of claims 5-12, wherein the 4-1BB costimulatory domain comprises the amino acid sequence of SEQ ID NO: 29.

18. The MAGE-A4-specific CAR of any one of claims 5-10 and 12-14, wherein the hinge comprises the amino acid sequence of SEQ ID NO: 34.

19. The MAGE-A4-specific CAR of any one of claims 5-10, 12-14, and 18, wherein the transmembrane domain comprises the amino acid sequence of SEQ ID NO: 36.

20. The MAGE-A4-specific CAR of any one of claims 5-10, and 12-14, 18 and 19, wherein the CD28 costimulatory domain comprises the amino acid sequence of SEQ ID NO: 38.

21. The MAGE-A4-specific CAR of any one of claims 5-20, wherein the signaling domain comprises a CD3zeta signaling domain.

22. The MAGE-A4-specific CAR of claim 21, wherein the CD3zeta signaling domain comprises the amino acid sequence of SEQ ID NO: 30.

23. The antigen-binding protein of any one of claims 1-4, wherein said antigen-binding protein is a MAGE-A4-specific antibody, or an antigen-binding fragment thereof.

24. The antigen-binding protein of any one of claims 1-4 and 23, or the MAGE-A4-specific CAR of any one of claims 5-22, wherein said antigen-binding protein or MAGE-A4-specific CAR comprises three heavy chain CDRs (HCDR1, HCDR2, and HCDR3) contained within a HCVR comprising the amino acid sequence set forth in SEQ ID NO: 2 or SEQ ID NO: 83.

25. The antigen-binding protein or the MAGE-A4-specific CAR of claim 24, wherein HCDR1 comprises the amino acid sequence set forth in SEQ ID NO: 4, HCDR2 comprises the amino acid sequence set forth in SEQ ID NO: 6, and HCDR3 comprises the amino acid sequence set forth in SEQ ID NO: 8.

26. The antigen-binding protein or the MAGE-A4-specific CAR of claim 25, wherein said HCVR comprises the amino acid sequence set forth in SEQ ID NO: 2.

27. The antigen-binding protein or the MAGE-A4-specific CAR of claim 25, wherein said HCVR comprises the amino acid sequence set forth in SEQ ID NO: 83.

28. The antigen-binding protein of any one of claims 1-4 and 23, or the MAGE-A4-specific CAR of any one of claims 5-22, wherein said antigen-binding protein or MAGE-A4-specific CAR comprises three heavy chain CDRs (HCDR1, HCDR2, and HCDR3) contained within a HCVR comprising the amino acid sequence set forth in SEQ ID NO: 107.

28. The antigen-binding protein or the MAGE-A4-specific CAR of claim 28, wherein HCDR1 comprises the amino acid sequence set forth in SEQ ID NO: 109, HCDR2 comprises the amino acid sequence set forth in SEQ ID NO: 111, and HCDR3 comprises the amino acid sequence set forth in SEQ ID NO: 113.

29. The antigen-binding protein or the MAGE-A4-specific CAR of claim 28, wherein said HCVR comprises the amino acid sequence set forth in SEQ ID NO: 107.

30. The antigen-binding protein of any one of claims 1-4 and 23, the MAGE-A4-specific CAR of any one of claims 5-22, or the antigen-binding protein or the MAGE-A4-specific CAR of any one of claims 24-29, wherein said antigen-binding protein or MAGE-A4-specific CAR comprises three light chain CDRs (LCDR1, LCDR2, and LCDR3) contained within a LCVR comprising the amino acid sequence set forth in SEQ ID NO: 10 or SEQ ID NO: 115.

31. The antigen-binding protein or the MAGE-A4-specific CAR of claim 30, wherein LCDR1 comprises the amino acid sequence set forth in SEQ ID NO: 12, LCDR2 comprises the amino acid sequence set forth in SEQ ID NO: 14, and LCDR3 comprises the amino acid sequence set forth in SEQ ID NO: 16.

32. The antigen-binding protein or the MAGE-A4-specific CAR of claim 31, wherein said LCVR comprises the amino acid sequence set forth in SEQ ID NO: 10.

33. The antigen-binding protein or the MAGE-A4-specific CAR of claim 30, wherein LCDR1 comprises the amino acid sequence set forth in SEQ ID NO: 117, LCDR2 comprises the amino acid sequence set forth in SEQ ID NO: 14, and LCDR3 comprises the amino acid sequence set forth in SEQ ID NO: 119.

34. The antigen-binding protein or the MAGE-A4-specific CAR of claim 33, wherein said LCVR comprises the amino acid sequence set forth in SEQ ID NO: 115.

35. The antigen-binding protein of any one of claims 1-4 and 23, or the MAGE-A4-specific CAR of any one of claims 5-22, wherein said antigen-binding protein or MAGE-A4-specific CAR comprises a HCVR that comprises the amino acid sequence set forth in SEQ ID NO: 2 and a LCVR that comprises the amino acid sequence set forth in SEQ ID NO: 10.

36. The antigen-binding protein of any one of claims 1-4 and 23, or the MAGE-A4-specific CAR of any one of claims 5-22, wherein said antigen-binding protein or MAGE-A4-specific CAR comprises a HCVR that comprises the amino acid sequence set forth in SEQ ID NO: 83 and a LCVR that comprises the amino acid sequence set forth in SEQ ID NO: 10.

37. The antigen-binding protein of any one of claims 1-4 and 23, or the MAGE-A4-specific CAR of any one of claims 5-22, wherein said antigen-binding protein or MAGE-A4-specific CAR comprises a HCVR that comprises the amino acid sequence set forth in SEQ ID NO: 107 and a LCVR that comprises the amino acid sequence set forth in SEQ ID NO: 115.

38. The MAGE-A4-specific CAR of claim 5, comprising the amino acid sequence of SEQ ID NO: 22.

39. The MAGE-A4-specific CAR of claim 5, comprising the amino acid sequence of SEQ ID NO: 105.

40. The MAGE-A4-specific CAR of claim 5, comprising the amino acid sequence of SEQ ID NO: 120.

41. The MAGE-A4-specific CAR of claim 5, comprising the amino acid sequence of SEQ ID NO: 121.

42. The antigen-binding protein of any one of claims 1-4 and 23, the MAGE-A4-specific CAR of any one of claims 5-22 and 38-41, or the antigen-binding protein or the MAGE-A4-specific CAR of any one of claims 24-37, wherein said antigen-binding protein or MAGE-A4-specific CAR specifically binds to one or more amino acids at positions 286-294 of SEQ ID NO: 32.

43. The antigen-binding protein of any one of claims 1-4 and 23, the MAGE-A4-specific CAR of any one of claims 5-22 and 38-41, or the antigen-binding protein or the MAGE-A4-specific CAR of any one of claims 24-37 and 42, wherein said antigen-binding protein or MAGE-A4-specific CAR interacts with one or more amino acids of said HLA.

44. The antigen-binding protein of any one of claims 1-4 and 23, the MAGE-A4-specific CAR of any one of claims 5-22 and 38-41, or the antigen-binding protein or the MAGE-A4-specific CAR of any one of claims 24-37, 42 and 43, wherein said HLA is HLA-A2.

45. An isolated nucleic acid molecule encoding the antigen-binding protein of any one of claims 1-4 and 23, the MAGE-A4-specific CAR of any one of claims 5-22 and 38-41, or the antigen-binding protein or the MAGE-A4-specific CAR of any one of claims 24-37 and 42-44.

46. The isolated nucleic acid molecule of claim 45, comprising a nucleotide sequence of SEQ ID NO: 21.

47. The isolated nucleic acid molecule of claim 45, comprising a nucleotide sequence of SEQ ID NO: 104.

48. A vector comprising the nucleic acid molecule of any one of claims 45-47.

49. The vector of claim 48, wherein the vector is a DNA vector, an RNA vector, a plasmid, a lentivirus vector, an adenovirus vector, or a retroviral vector.

50. The vector of claim 49, wherein the vector is a lentivirus vector.

51. A cell comprising the nucleic acid molecule of any one of claims 45-47, or a vector of any one of claims 48-50.

52. The cell of claim 51, wherein the cell is a human T cell.

53. An engineered cell comprising an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44.

54. The engineered cell of claim 53 that is an immune cell.

55. The engineered cell of claim 54, wherein the immune cell is an immune effector cell.

56. The engineered cell of claim 55, wherein the immune effector cell is a T lymphocyte.

57. The engineered cell of claim 56, wherein the T lymphocyte is an inflammatory T lymphocyte, a cytotoxic T lymphocyte, a regulatory T lymphocyte, or a helper T lymphocyte.

58. The engineered cell of claim 57 that is a CD8+ cytotoxic T lymphocyte.

59. The engineered cell of any one of claims 53-58 for use in the treatment of a MAGE-A4-expressing cancer.

60. The engineered cell of claim 59, wherein the MAGE-A4-expressing cancer is multiple myeloma.

61. The engineered cell of claim 59, wherein the MAGE-A4-expressing cancer is melanoma.

62. An engineered human T cell comprising a chimeric antigen receptor comprising, from N-terminus to C-terminus: (a) an extracellular ligand-binding domain comprising an anti-MAGE-A4 single chain variable fragment (scFv) domain comprising a light chain variable region (LCVR) and a heavy chain variable region (HCVR); (b) a hinge; (c) a transmembrane domain; and (d) a cytoplasmic domain comprising a 4-1BB costimulatory domain or a CD28 costimulatory domain and a CD3zeta signaling domain, wherein the LCVR comprises complementarity determining regions (CDRs) of a LCVR comprising the amino acid sequence of SEQ ID NO: 10 or SEQ ID NO: 115, and CDRs of a HCVR comprising an amino acid sequence of SEQ ID NO: 2, SEQ ID NO: 83 or SEQ ID NO: 107.

63. The engineered human T cell of claim 62, wherein said anti-MAGE-A4 scFv specifically binds to one or more amino acid residues of positions 286-294 of SEQ ID NO: 32.

64. The engineered human T cell of claim 62 or 63, wherein the scFv domain comprises a HCVR/LCVR amino acid sequence pair comprising the amino acid sequences of SEQ ID NOs: 2/10.

65. The engineered human T cell of claim 62 or 63, wherein the scFv domain comprises a HCVR/LCVR amino acid sequence pair comprising the amino acid sequences of SEQ ID NOs: 2/83.

66. The engineered human T cell of claim 62 or 63, wherein the scFv domain comprises a HCVR/LCVR amino acid sequence pair comprising the amino acid sequences of SEQ ID NOs: 107/115.

67. The engineered human T cell of claim 64 or 65, wherein the HCVR comprises three heavy chain CDRs (HCDR1, HCDR2, and HCDR3) and the LCVR comprises three light chain CDRs (LCDR1, LCDR2, and LCDR3), wherein HCDR1 comprises the amino acid sequence set forth in SEQ ID NO: 4, HCDR2 comprises the amino acid sequence set forth in SEQ ID NO: 6, HCDR3 comprises the amino acid sequence set forth in SEQ ID NO: 8, LCDR1 comprises the amino acid sequence set forth in SEQ ID NO: 12, LCDR2 comprises the amino acid sequence set forth in SEQ ID NO: 14, and LCDR3 comprises the amino acid sequence set forth in SEQ ID NO: 16.

68. The engineered human T cell of claim 66, wherein the HCVR comprises three heavy chain CDRs (HCDR1, HCDR2, and HCDR3) and the LCVR comprises three light chain CDRs (LCDR1, LCDR2, and LCDR3), wherein HCDR1 comprises the amino acid sequence set forth in SEQ ID NO: 109, HCDR2 comprises the amino acid sequence set forth in SEQ ID NO: 111, HCDR3 comprises the amino acid sequence set forth in SEQ ID NO: 113, LCDR1 comprises the amino acid sequence set forth in SEQ ID NO: 117, LCDR2 comprises the amino acid sequence set forth in SEQ ID NO: 14, and LCDR3 comprises the amino acid sequence set forth in SEQ ID NO: 119.

69. The engineered human T cell of any one of claims 62-68, wherein the hinge comprises the amino acid sequence of SEQ ID NO: 27.

70. The engineered human T cell of any one of claims 62-69, wherein the transmembrane domain comprises the amino acid sequence of SEQ ID NO: 28.

71. The engineered human T cell of any one of claims 62-70, wherein the 4-1BB costimulatory domain comprises the amino acid sequence of SEQ ID NO: 29.

72. The engineered human T cell of any one of claims 62-68, wherein the hinge comprises the amino acid sequence of SEQ ID NO: 34.

73. The engineered human T cell of any one of claims 62-68 and 72, wherein the transmembrane domain comprises the amino acid sequence of SEQ ID NO: 36.

74. The engineered human T cell of any one of claims 62-68, 72 and 73, wherein the CD28 costimulatory domain comprises the amino acid sequence of SEQ ID NO: 38.

75. The engineered human T cell of any one of claims 62-74, wherein the CD3zeta signaling domain comprises the amino acid sequence of SEQ ID NO: 30.

76. The engineered human T cell of claim 62, comprising a chimeric antigen receptor comprising the amino acid sequence of SEQ ID NO: 22.

77. The engineered human T cell of claim 62, comprising a chimeric antigen receptor comprising the amino acid sequence of SEQ ID NO: 105.

78. The engineered human T cell of claim 62, comprising a chimeric antigen receptor comprising the amino acid sequence of SEQ ID NO: 120.

79. The engineered human T cell of claim 62, comprising a chimeric antigen receptor comprising the amino acid sequence of SEQ ID NO: 121.

80. A pharmaceutical composition comprising a genetically-modified human T cell and a pharmaceutically acceptable carrier, wherein the genetically-modified human T cell comprises an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44.

81. A pharmaceutical composition comprising the engineered cell of any one of claims 53-58 and a pharmaceutically acceptable carrier.

82. A pharmaceutical composition comprising the engineered human T cell of any one of claims 62-79 and a pharmaceutically acceptable carrier.

83. The pharmaceutical composition of any one of claims 80-82 for use in the treatment of a MAGE-A4-expressing cancer.

84. The pharmaceutical composition of claim 83, wherein the MAGE-A4-expressing cancer is multiple myeloma.

85. The pharmaceutical composition of claim 83, wherein the MAGE-A4-expressing cancer is melanoma.

86. Use of the antigen-binding protein of any one of claims 1-4 and 23, the MAGE-A4-specific CAR of any one of claims 5-22 and 38-41, or the antigen-binding protein or the MAGE-A4-specific CAR of any one of claims 24-37 and 42-44, the nucleic acid molecule of any one of claims 45-47, the vector of any one of claims 48-50, the cell of claim 51 or 52, the engineered cell of any one of claims 53-58, or the engineered human T cell of any one of claims 62-79 in the manufacture of a medicament for the treatment of a MAGE-A4-expressing cancer.

87. The use of claim 86, wherein the MAGE-A4-expressing cancer is multiple myeloma.

88. The use of claim 86, wherein the MAGE-A4-expressing cancer is melanoma.

89. A method of enhancing T lymphocyte activity in a subject comprising, introducing into the subject a T lymphocyte comprising an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44.

90. A method for treating a subject suffering from cancer, comprising introducing into the subject a therapeutically effective amount of a T lymphocyte comprising an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44.

91. A method for stimulating a T cell-mediated immune response to a target cell population or tissue in a subject comprising, administering to the subject an effective amount of a cell genetically modified to express an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44.

92. A method of providing anti-tumor immunity in a subject, the method comprising administering to the subject an effective amount of a cell genetically modified to express an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44.

93. The method of any one of claims 89-92, wherein the subject is a human.

94. The method of any one of claims 89-93, wherein the subject has multiple myeloma, synovial sarcoma, esophageal cancer, head and neck cancer, lung cancer, bladder cancer, ovarian cancer, uterine cancer, stomach cancer, cervical cancer, breast cancer, or melanoma.

95. The method of claim 94, wherein the subject has multiple myeloma.

96. A method of engineering a population of cells to express an antigen-binding protein that specifically binds an HLA-bound MAGE-A4 or a MAGE-A4-specific chimeric antigen receptor (CAR), comprising:
(a) introducing into a population of immune cells nucleic acid molecules encoding an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44;
(b) culturing said population of immune cells under conditions to express said nucleic acid molecules; and
(c) isolating said immune cells expressing said MAGE-A4-specific antigen-binding protein at the cells' surface.

97. The method of claim 96, further comprising obtaining said population of immune cells from a subject prior to introducing said nucleic acid molecule.

98. A method of treating a MAGE-A4-expressing cancer in a subject, comprising:
(a) engineering a population of cells according to claim 96 or claim 97; and
(b) reintroducing said population of cells expressing said chimeric antigen receptor into said subject.

99. The method of claim 98, wherein said MAGE-A4-expressing cancer is multiple myeloma.

100. An isolated antigen-binding protein, wherein the antigen-binding protein comprises a first antigen-binding domain that binds specifically to an HLA-bound Melanoma-Associated Antigen A4 (MAGE-A4) and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (A1-HCVR) and three light chain CDRs (A1-LCDR1, A1-LCDR2 and A1-LCDR3) contained in a light chain variable region (A1-LCVR), and the second antigen-binding domain comprises three heavy chain CDRs (A2-HCDR1, A2-HCDR2 and A2-HCDR3) contained in a heavy chain variable region (A2-HCVR) and three light chain CDRs (A2-LCDR1, A2-LCDR2 and A2-LCDR3) contained in a light chain variable region (A2-LCVR), wherein A1-HCVR comprises the amino acid sequence of SEQ ID NO: 2, A1-LCVR comprises the amino acid sequence of SEQ ID NO: 10, A2-HCVR comprises the amino acid sequence of SEQ ID NO: 55, and A2-LCVR comprises the amino acid sequence of SEQ ID NO: 10.

101. An isolated antigen-binding protein, wherein the antigen-binding protein comprises a first antigen-binding domain that binds specifically to an HLA-bound Melanoma-Associated Antigen A4 (MAGE-A4) and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (A1-HCVR) and three light chain CDRs (A1-LCDR1, A1-LCDR2 and A1-LCDR3) contained in a light chain variable region (A1-LCVR), and the second antigen-binding domain comprises three heavy chain CDRs (A2-HCDR1, A2-HCDR2 and A2-HCDR3) contained in a heavy chain variable region (A2-HCVR) and three light chain CDRs (A2-LCDR1, A2-LCDR2 and A2-LCDR3) contained in a light chain variable region (A2-LCVR), wherein A1-HCVR comprises the amino acid sequence of SEQ ID NO: 2, A1-LCVR comprises the amino acid sequence of SEQ ID NO: 10, A2-HCVR comprises the amino acid sequence of SEQ ID NO: 73, and A2-LCVR comprises the amino acid sequence of SEQ ID NO: 10.

102. The isolated antigen-binding protein of claim 100 or claim 101, wherein said antigen-binding protein interacts with amino acids 286-294, or a portion thereof, of SEQ ID NO: 32.

103. The isolated antigen-binding protein of claim 100 or 101, wherein said isolated antigen-binding protein is a CAR.

104. The isolated antigen-binding protein of claim 100 or 101, wherein said isolated antigen-binding protein is a bispecific antibody.

105. The isolated antigen-binding protein of any one of claims 100-104, wherein said isolated antigen-binding protein interacts with amino acids 286-294, or a portion thereof, of SEQ ID NO: 32.

106. The isolated antigen-binding protein of any one of claims 100-105, wherein said isolated antigen-binding protein interacts with CD3.

107. The isolated antigen-binding protein of claim 100, or any one of claims 102-106, wherein the isolated antigen binding protein comprises a heavy chain variable region (HCVR) comprising an amino acid sequence having at least 95% sequence identity to SEQ ID NO: 2, or SEQ ID NO: 83.

108. The isolated antigen-binding protein of claim 100, or any one of claims 102-106, wherein the isolated antigen binding protein comprises a heavy chain variable region (HCVR) comprising an amino acid sequence having at least 95% sequence identity to SEQ ID NO: 55.

109. The isolated antigen-binding protein of any one of claims 101-106, wherein the isolated antigen binding protein comprises a heavy chain variable region (HCVR) comprising an amino acid sequence having at least 95% sequence identity to SEQ ID NO: 73.

110. An isolated antigen-binding protein that binds to the same epitope as an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44.

111. An isolated antigen-binding protein that competes for binding with an antigen-binding protein of any one of claims 1-4 and 23, a MAGE-A4-specific CAR of any one of claims 5-22 and 38-41, or an antigen-binding protein or the MAGE-A4-specific CAR of any one of claims 24-37 and 42-44.

112. The isolated antigen-binding protein of claim 110 or 111, wherein said isolated antigen-binding protein is a CAR.

113. The isolated antigen-binding protein of claim 110 or 111, wherein said isolated antigen-binding protein is a bispecific antibody.

114. The isolated antigen-binding protein of any one of claims 111-113, wherein said isolated antigen-binding protein interacts with amino acids 286-294, or a portion thereof, of SEQ ID NO: 32.

115. The isolated antigen-binding protein of claim 113 or 114, wherein said isolated antigen-binding protein interacts with CD3.

116. The isolated antigen-binding protein of any one of claims 100-115, wherein the isolated antigen binding protein comprises a heavy chain variable region (HCVR) comprising three heavy chain CDRs, HCDR1, HCDR2 and HCDR3, comprising the amino acid sequences of SEQ ID NOs: 4, 6 and 8, respectively.

117. The isolated antigen-binding protein of any one of claim 100 or 102-116, wherein the isolated antigen binding protein comprises a HCVR corresponding to another arm of the bispecific antibody comprising three heavy chain CDRs, HCDR1, HCDR2 and HCDR3, comprising the amino acid sequences of SEQ ID NOs: 57, 59 and 61, respectively.

118. The isolated antigen-binding protein of any one of claims 101-116, wherein the isolated antigen binding protein comprises a HCVR corresponding to another arm of the bispecific antibody comprising three heavy chain CDRs comprising the amino acid sequences of SEQ ID NOs: 75, 77 and 79, respectively.

119. The isolated antigen-binding protein of any one of claims 100-118, wherein the isolated antigen binding protein comprises a light chain variable region (LCVR) comprising an amino acid sequence having at least 95% sequence identity to SEQ ID NO: 10 and/or an LCVR comprising an amino acid sequence having at least 95% sequence identity to SEQ ID NO: 63.

120. An isolated recombinant antibody or antigen-binding fragment thereof that specifically binds to a Melanoma-Associated Antigen A4 (MAGE-A4) polypeptide, wherein the antibody has one or more of the following characteristics:
(a) binds to the MAGE-A4 polypeptide with an EC50 of less than about 2×10⁻⁹ M;
(b) demonstrates an ability to reduce tumor cell viability as compared to isolated recombinant antibodies that do not specifically bind the MAGE-A4 polypeptide; and/or
(c) comprises (i) three heavy chain complementarity determining regions (CDRs) (HCDR1, HCDR2, and HCDR3) contained within a heavy chain variable region (HCVR) comprising an amino acid sequence having at least about 90% sequence identity to an HCVR set forth in Table 1; and (ii) three light chain CDRs (LCDR1, LCDR2, and LCDR3) contained within a light chain variable region (LCVR) comprising the amino acid sequence having at least about 90% sequence identity to an LCVR set forth in Table 1.

121. The antibody or antigen-binding fragment of claim 120, wherein the MAGE-A4 polypeptide is an HLA-A2 bound MAGE-A4 polypeptide.

122. The isolated antibody or antigen-binding fragment thereof of claim 120 or 121, comprising an HCVR having an amino acid sequence of SEQ ID NO: 2, SEQ ID NO: 83, or SEQ ID NO: 107.

123. The isolated antibody or antigen-binding fragment thereof of any one of claims 120-122, comprising an LCVR having an amino acid sequence of SEQ ID NO: 10 or SEQ ID NO: 115.

124. The isolated antibody or antigen-binding fragment thereof of any one of claims 120-123, comprising an HCVR/LCVR amino acid sequence pair of SEQ ID NOs: 2/10, or SEQ ID NOs: 83/10, or SEQ ID NOs: 107/115.

125. An isolated antibody or antigen-binding fragment thereof of any one of claims 120-124, comprising:
(a) an HCDR1 domain having an amino acid sequence of SEQ ID NO: 4;
(b) an HCDR2 domain having an amino acid sequence of SEQ ID NO: 6;
(c) an HCDR3 domain having an amino acid sequence of SEQ ID NO: 8;
(d) an LCDR1 domain having an amino acid sequence of SEQ ID NO: 12;
(e) an LCDR2 domain having an amino acid sequence of SEQ ID NO: 14; and
(f) an LCDR3 domain having an amino acid sequence of SEQ ID NO: 16.

126. An isolated antibody or antigen-binding fragment thereof of any one of claims 120-124, comprising:
(a) an HCDR1 domain having an amino acid sequence of SEQ ID NO: 109;
(b) an HCDR2 domain having an amino acid sequence of SEQ ID NO: 111;
(c) an HCDR3 domain having an amino acid sequence of SEQ ID NO: 113;
(d) an LCDR1 domain having an amino acid sequence of SEQ ID NO: 117;
(e) an LCDR2 domain having an amino acid sequence of SEQ ID NO: 14; and
(f) an LCDR3 domain having an amino acid sequence of SEQ ID NO: 119.

127. The isolated antibody or antigen-binding fragment thereof of any one of claims 120-126 which is an IgG1 antibody.

128. The isolated antibody or antigen-binding fragment thereof of any one of claims 120-126 which is an IgG4 antibody.

129. The isolated antibody or antigen-binding fragment thereof of any one of claims 120-128 which is a bispecific antibody.

130. An isolated recombinant antibody or antigen-binding fragment thereof comprising a first antigen-binding domain that specifically binds to a Melanoma-Associated Antigen A4 (MAGE-A4) polypeptide and a second antigen-binding domain that specifically binds to a CD3 polypeptide, wherein:
(a) the first antigen-binding domain (A1) that binds MAGE-A4 comprises three heavy chain complementarity determining regions (A1-HCDR1, A1-HCDR2, and A1-HCDR3) and three light chain complementarity determining regions (A1-LCDR1, A1-LCDR2, and A1-LCDR3), wherein
A1-HCDR1 comprises the amino acid sequence of SEQ ID NO: 4 or SEQ ID NO: 109;
A1-HCDR2 comprises the amino acid sequence of SEQ ID NO: 6 or SEQ ID NO: 111;
A1-HCDR3 comprises the amino acid sequence of SEQ ID NO: 8 or SEQ ID NO: 113;
A1-LCDR1 comprises the amino acid sequence of SEQ ID NO: 12, SEQ ID NO: 65, or SEQ ID NO: 117;
A1-LCDR2 comprises the amino acid sequence of SEQ ID NO: 14;
A1-LCDR3 comprises the amino acid sequence of SEQ ID NO: 16, SEQ ID NO: 67, or SEQ ID NO: 119; and
(b) the second antigen-binding domain (A2) that binds CD3 comprises three heavy chain complementarity determining regions (A2-HCDR1, A2-HCDR2, and A2-HCDR3) and three light chain complementarity determining regions (A2-LCDR1, A2-LCDR2, and A2-LCDR3), wherein
A2-HCDR1 comprises the amino acid sequence of SEQ ID NO: 57 or SEQ ID NO: 75;
A2-HCDR2 comprises the amino acid sequence of SEQ ID NO: 59 or SEQ ID NO: 77;
A2-HCDR3 comprises the amino acid sequence of SEQ ID NO: 61 or SEQ ID NO: 79;
A2-LCDR1 comprises the amino acid sequence of SEQ ID NO: 12, SEQ ID NO: 65, or SEQ ID NO: 117;
A2-LCDR2 comprises the amino acid sequence of SEQ ID NO: 14;
A2-LCDR3 comprises the amino acid sequence of SEQ ID NO: 16, SEQ ID NO: 67, or SEQ ID NO: 119.

131. A pharmaceutical composition comprising the isolated antibody or antigen-binding fragment thereof of any one of claims 120-130 and a pharmaceutically acceptable carrier or diluent.

132. The pharmaceutical composition of claim 131, wherein said pharmaceutical composition further comprises a second therapeutic agent.

133. The pharmaceutical composition of claim 132, wherein said second therapeutic agent is selected from the group consisting of: an anti-tumor agent, steroids, and targeted therapies.

134. A polynucleotide molecule comprising a polynucleotide sequence that encodes one or more HCVRs and/or one or more LCVRs of an antibody as set forth in any one of claims 120-133.

135. A vector comprising the polynucleotide of claim 134.

136. A cell comprising the vector of claim 135.

137. A method of treating a MAGE-A4 expressing cancer, the method comprising administering an antibody or antigen-binding fragment of any one of claims 120-130, or the pharmaceutical composition of any one of claims 131-133, to a subject.

138. The method of claim 137, wherein said pharmaceutical composition is administered in combination with a second therapeutic agent.

139. The method of claim 138, wherein said second therapeutic agent is selected from the group consisting of: an anti-tumor agent, steroids, and targeted therapies.
